(19) United States
(12) Patent Application Publication
(10) Pub. No.: US 2023/0331852 A1
(43) Pub. Date: Oct. 19, 2023

(54) ANTI-CLEC-1A ANTIBODIES AND ANTIGEN-BINDING FRAGMENT THEREOF

(71) Applicants: OSE IMMUNOTHERAPEUTICS, NANTES (FR); NANTES UNIVERSITE, NANTES (FR); INSTITUT NATIONAL DE LA SANTE ET DE LA RECHERCHE MEDICALE, PARIS (FR)

(72) Inventors: Elise CHIFFOLEAU, NANTES (FR); Nicolas POIRIER, GRANDCHAMPS DES FONTAINES (FR); Ariane DESSELLE, NANTES (FR); Virginie THEPENIER, SAINTE-PAZANNE (FR); Caroline MARY, SAINTE-PAZANNE (FR); Vanessa GAUTTIER, REZE (FR); Sabrina PENGAM, ST LUCE S/LOIRE (FR)

(21) Appl. No.: 17/779,428

(22) PCT Filed: Dec. 4, 2020
(86) PCT No.: PCT/EP2020/084770

§ 371 Date: May 24, 2022

(30) Foreign Application Priority Data
Dec. 5, 2019 (EP) 19306583.6

(51) Int. Cl. C07K 16/28; A61P 35/00

(52) U.S. Cl. CPC C07K 16/2851; A61P 35/00; C07K 2317/565; C07K 2317/92

(57) ABSTRACT

The invention pertains to the field of immunotherapy. The present invention provides new specific anti-CLEC-1A compounds, in particular antibodies. The compounds of the invention are able to specifically binds to CLEC-1A receptor and antagonize the binding of CLEC-1A to its endogenous ligand(s). The use of the compounds of the invention may be useful for treating deleterious conditions.

### FIELD OF THE INVENTION

The invention pertains to the field of immunotherapy. The present invention provides new specific anti-CLEC-1A compounds, in particular antibodies. The compounds of the invention are able to specifically bind to CLEC-1A and are antagonist of human CLEC-1A, in particular antagonize the binding of CLEC-1A to at least one of its ligand(s), particularly its endogenous ligand(s). The use of the compounds of the invention may be useful for treating deleterious conditions, including but not limited to cancers.

### BACKGROUND OF THE INVENTION

Immunotherapy treatments harnessing the patient's immune system herald a new era of personalized medicine, offering hope for curative responses in patients with serious illnesses. Cell-mediated immunity can eliminate or prevent diseases, like but not limited to cancers, autoimmune disease and allergic diseases. Recent developments in therapies include cell engineering, disease targeting and modulation of the immune system of the patients to provide a more focused and effective response to diseases. Among these strategies, immunotherapy with immune checkpoint inhibitors or activators has become an essential weapon against these diseases, most particularly for the treatment against cancers. These molecules, often expressed by immune system cells, such as T cells or dendritic cells but also by some cancer cells, enhance the immune response to the patient and keep or initiate immune cell response against pathogenic cells. Immune checkpoints refer to a plethora of inhibitory pathways hardwired into the immune system that are crucial for maintaining self-tolerance and minimize collateral tissue damage.

C-type lectin receptors (CLRs) are a large family of transmembrane and soluble receptors. These receptors contain one or more carbohydrate-recognition domain able to recognize a wide variety of glycans on pathogens or on self-proteins. For these receptors, glycan recognition is dependent from Ca²⁺. Many related-CLRs are nonetheless able to recognize carbohydrates but independently of Ca²⁺; these receptors are referred to C-type lectin-like receptors (CTLRs). These receptors are of particular interest for their role in coupling both innate and adaptive immunity. CTLRs are expressed mostly by cells of myeloid lineage such as monocytes, macrophages, dendritic cells (DCs), and neutrophils. CTLRs not only serve as antigen-uptake receptors for internalization and presentation to T cells but also trigger multiple signalling pathways leading to NF-κB, type I interferon (IFN), and/or inflammasome activation. By their capacity to present antigen and ensure the balance between cellular activation and suppression, CTLRs have emerged as challenging pharmacological targets to treat a wide variety of diseases including cancers, autoimmune diseases or allergy. CTLR modulation seems to represent a promising strategy for disease management although attempts at identifying endogenous ligands as well as efforts to elucidate their role in immunity are still warrant.

Among these CTLRs, a particular member named CLEC-1, but also referenced under the acronyms CLEC1, CLEC1A, CLEC-1A, CLEC1 receptor, CLEC receptor and CLEC-1A receptor is of particular interest. Although the C-type lectin-like receptor-1 (CLEC-1) is identified for several years, the downstream signalling and ligand(s) remain uncharacterized. In human and rodent, CLEC1 is expressed by myeloid cells such as monocytes, DC, and macrophages but also by endothelial cells. CLEC-1 expression is decreased by pro-inflammatory stimuli and is enhanced by TGFβ. Interestingly, CLEC-1 was found to be expressed mostly intracellular particularly in human endothelial cells and neutrophils, suggesting the requirement of particular conditions for cell-surface expression.

Recently, the present inventors showed for the first time that CLEC-1A is expressed at the cell-surface by conventional DCs (cDCs) and by small subsets of monocytes and DCs in human blood and is enhanced by the immunosuppressive cytokine TGFβ (see international application No. Wo2018073440). The inventors showed that human CLEC-1A is expressed by M2-type pro-tumoral macrophages, by myeloid cells from pleural effusion mesothelioma and from ovarian tumor ascites. They demonstrated in both rodent and human that CLEC-1 acts as an inhibitory receptor in myeloid cells and prevent IL12p40 expression and downstream Thl and Th17 in vivo responses.

They also showed that human T cells proliferation and human IFN-gamma are increased using anti-hCLEC-1A antibody as antagonist of CLEC-1A. They also demonstrated that mice deficient in CLEC-1 are better resistant to tumor growth and exhibit an increased survival rate in a hepatocarcinoma mice model. Therefore, CLEC-1A as a cell-surface receptor may represent a useful therapeutic tool to enhance anti-tumor immunity in a clinical setting.

In this context, the inventors provide for the first time anti-CLEC-1A compounds, in particular anti-CLEC-1A antibodies, which recognize and bind specifically to the extracellular domain of human CLEC-1A, which are antagonist of human CLEC-1A, in particular which are suitable for antagonizing the binding of the CLEC-1A to at least one of its ligand, particularly an endogenous ligand, and correlate when used in vivo and/or in vitro with a modulation, in particular an increase, of the phagocytosis of tumor cells and/or secondary necrotic cells by myeloid cells, in particular by dendritic cells and/or macrophages.

As shown in the examples of the invention, it is provided for the first-time anti-CLEC-1A compounds, in particular anti-CLEC-1A antibodies, that have the capability to correlate when used in vivo and/or in vitro with a modulation, in particular an increase, of the phagocytosis of tumor cells and/or secondary necrotic cells by myeloid cells, in particular by dendritic cells and/or macrophages. By contrast to the anti-CLEC-1A antibody disclosed in the prior art (WO 2018/073440A1 and the article of Robles et al. (Blood advances 2017)), which is used in some of the working examples of the invention that binds to CLEC-1A and which is an antagonist of human CLEC-1A, it is illustrated in the present description that the antibodies according to any embodiment of the invention correlate with a modulation, in particular an increase, of the phagocytosis of tumor cells by cells of the immune system when used in vitro. Tumor cells and/or secondary necrotic cells interacting with CLEC-1A escape phagocytosis by CLEC-1A-expressing myeloid cells. The antibodies of the invention interact with CLEC-1A in a manner that prevents functional interaction between CLEC-1A and tumor cells and/or secondary necrotic cells usually interacting with CLEC-1A-expressing cells, such functional interaction preventing the tumor cells to escape phagocytosis. As illustrated in the present invention, the antagonist anti-CLEC-1A antibody disclosed in the prior art (WO 2018/073440A1 and the article of Robles et al. (Blood advances 2017)), which is used in some of the examples of the present invention does not correlate with a modulation of the phagocytosis of tumor cells by myeloid cells, in particular by dendritic cells and/or macrophages. Modulation of the phagocytosis of tumor cells is only illustrated when a compound according to the invention is present in the examples. CLEC-1A-expressing myeloid cells, in particular CLEC-1A-expressing dendritic cells and/or macrophages, are not prevented to exert their phagocytosis capabilities of tumor cells and/or secondary necrotic cells when an antagonist compound according to the invention is present. Several very advantageous biological effects are reached when the antagonist compounds of the invention are administered, associated in particular with the phagocytosis capability of myeloid cells, including dendritic cells and/or macrophages. The antibodies of the invention which are suitable antagonists of CLEC-1A correlate with the modulation, in particular with the increase, of the phagocytosis capability of dendritic cells and/or macrophages, like activated macrophages. The administration of the anti-CLEC1A compounds, in particular anti-CLEC-1A antibodies of the invention correlate with enhanced phagocytosis of tumor cells and/or cancer cells and/or secondary necrotic cells by dendritic cells and/or macrophages by antagonizing the binding of the CLEC-1A to its target(s) (at least one of its ligand) expressed by tumor cells. When CLEC-1A-expressing macrophages or dendritic cells interact with cells expressing one ligand of CLEC-1A, the phagocytosis capability of these macrophages or denditric cells is inhibited or reduced. Tumor cells that express a ligand of CLEC-1A escape phagocytosis exerted by macrophages and dendritic cells. As shown in the examples of the invention, when the anti-CLEC1A antibodies disclosed herein are administered, the inhibition of the phagocytosis capability of macrophages and of dendritic cells is removed by antagonizing the CLEC-1A interaction with the tumor cells, thereby leading to phagocytosis of tumor cells by macrophages and dendritic cells.

In addition to their effect on the phagocytosis of tumor cells and/or secondary necrotic cells by myeloid cells, the antibodies of the invention may also modulate, in particular enhance or increase, the proliferation of T cells and/or the activation of T cells.

The antibodies described herein may be efficiently produced in recombinant production systems, allowing the provision of chimeric or (fully) humanized antibodies exhibiting the functional features disclosed here above in a sufficient amount for further developments.

Besides, the antibodies of the invention have a specific affinity for the human CLEC-1A, as compared to its mice orthologue, since the antibodies of the invention do not cross-react with mice CLEC-1A protein in vitro. Moreover, as shown in the examples of the invention, the anti-CLEC-1A compounds, in particular anti-CLEC-1A antibodies, of the invention specifically bind to the extracellular domain of CLEC-1A expressed on the cell membrane of human cells in vitro.

In an embodiment of the invention, the antibodies of the invention disrupt the interaction between CLEC-1A expressed by myeloid cells, in particular by dendritic cells and/or macrophages, and secondary necrotic cells and/or tumor cells, like tumor cells present in a host having a cancer or developing a cancer, and/or with the intracellular content of secondary necrotic cells and/tumor cell. The present inventors determined that a ligand of CLEC-1A could be expressed or overexpressed, but not necessarily on the membrane on these cells, by damaged cells or tumor cells, and could therefore be involved in anti-tumor immunity and improve the death of tumor cells induced by the immune cells.

It is therefore provided antibodies, for which the inventors provide evidence, that they:
bind specifically to human CLEC-1A, in particular to CLEC-1A expressed on the cell membrane of human cells,
are antagonist of human CLEC-1A, in particular suitable for antagonizing the binding of the CLEC-1A to at least one of its ligands, particularly one of its endogenous ligands;
may be recovered at a significant yield allowing the provision of antibodies exhibiting the functional features disclosed here above in a sufficient amount for further developments; and
correlate when used in vivo and/or in vitro with a modulation, in particular an increase, of the phagocytosis of tumor cells and/or secondary necrotic cells by myeloid cells, in particular by dendritic cells and/or macrophages.

Such compounds are particularly suitable for their uses in the prevention and/or the treatment of several diseases or deleterious conditions, in particular wherein the phagocytosis exerted by dendritic cells and/or macrophages needs to be improved, more particularly for modulating the phagocytosis of tumor cells and/or secondary necrotic cells, preferably the phagocytosis activity by myeloid cells, in particular for improving the phagocytosis capability of dendritic cells and/or macrophages, to improve the outcome of the disease by increasing the phagocytosis of tumor cells by myeloid cells, in particular by dendritic cells and/or macrophages.

Such compounds may also be particularly suitable for their uses in the prevention and/or the treatment of several diseases, in particular for modulating the T cell response, in particular by enhancing the activation and/or the proliferation of T cells.

In a particular embodiment of the invention, the anti-CLEC-1A compound are suitable for decrease the overall number of myeloid-derived suppressor cells, thereby leading to a decrease of immunosuppressive cells, like but not limited to immunosuppressive myeloid cells.

### SUMMARY OF THE INVENTION

Accordingly, in a first aspect of the invention, it is disclosed an antibody or antigen-binding fragment thereof that specifically binds to the extracellular domain of human C-type lectin-like receptor-1 member A receptor (CLEC-1A receptor) which comprises:
an antibody heavy chain variable domain comprising three VHCDRs wherein their amino acid sequences are respectively selected from:
VHCDR1 of SEQ ID No: 57; SEQ ID No: 65; SEQ ID No: 73; SEQ ID No: 81; SEQ ID No: 89 or SEQ ID No: 97; in particular SEQ ID No: 65, SEQ ID No: 81 or SEQ ID No: 97; and
VHCDR2 of SEQ ID No: 59; SEQ ID No: 67; SEQ ID No: 75; SEQ ID No: 83; or SEQ ID No: 91; in particular SEQ ID No. 67; SEQ ID No: 75 or SEQ ID No: 83; and
VHCDR3 of SEQ ID No: 61; SEQ ID No: 69; SEQ ID No: 77; SEQ ID No: 85 or SEQ ID No: 93; in particular SEQ ID No. 69; SEQ ID No: 77 or SEQ ID No: 85; and
an antibody light chain variable domain comprising three VLCDRs wherein their amino acid sequence is selected from:
VLCDR1 of SEQ ID No: 4; SEQ ID No: 12; SEQ ID No: 20; SEQ ID No: 28 or SEQ ID No: 36; in particular SEQ ID No. 12; SEQ ID No: 20 or SEQ ID No: 28; and
VLCDR2 of SEQ ID No: 6; SEQ ID No: 14; SEQ ID No: 22; SEQ ID No: 30 or SEQ ID No: 38; in particular SEQ ID No. 14; SEQ ID No: 22; or SEQ ID No: 30 and
VLCDR3 of SEQ ID No: 8; SEQ ID No: 16; SEQ ID No: 24; SEQ ID No: 32 or SEQ ID No: 40; in particular SEQ ID No. 16; SEQ ID No: 24 or SEQ ID No: 32.

An antibody or an antigen-binding fragment thereof according to this embodiment is suitable for antagonizing human CLEC-1A while its binding property for this receptor is specific. Moreover, production in different cell lines, including but not limited to mammalian cell lines, with a yield of production suitable for purposes of development of a drug candidate is reached.

The inventors synthetized several anti-CLEC-1A antibodies, each comprising combinations of heavy chain variable domain CDRs and light chain variable domain CDRs. Accordingly, in a second aspect of the invention, it is provided an antibody or an antigen-binding fragment thereof, wherein
the antibody heavy chain variable domain comprises the VHCDR1, VHCDR2 and VHCDR3 of sequence:
SEQ ID No: 57; SEQ ID No: 59 and SEQ ID No: 61 respectively; or
SEQ ID No: 65; SEQ ID No: 67 and SEQ ID No: 69 respectively; or
SEQ ID No: 73; SEQ ID No: 75 and SEQ ID No: 77 respectively; or
SEQ ID No: 81; SEQ ID No: 83 and SEQ ID No: 85 respectively; or
SEQ ID No: 89; SEQ ID No: 91 and SEQ ID No: 93 respectively; or
SEQ ID No: 97; SEQ ID No: 75 and SEQ ID No: 77 respectively; or/ or wherein
the antibody light chain variable domain comprises the VLCDR1, VLCDR2 and VLCDR3 of sequence:
SEQ ID No: 4; SEQ ID No: 6 and SEQ ID No: 8 respectively; or
SEQ ID No: 12; SEQ ID No: 14 and SEQ ID No: 16 respectively; or
SEQ ID No: 20; SEQ ID No: 22 and SEQ ID No: 24 respectively; or
SEQ ID No: 28; SEQ ID No: 30 and SEQ ID No: 32 respectively; or
SEQ ID No: 36; SEQ ID No: 38 and SEQ ID No: 40 respectively.

In another aspect, the invention relates to an antibody or an antigen-binding fragment thereof, which specifically binds to the extracellular domain of human C-type lectin-like receptor-1 member A receptor (CLEC-1A receptor) and which correlates when used in vivo and/or in vitro with a modulation, in particular an increase, of the phagocytosis of tumor cells and/or secondary necrotic cells by myeloid cells, in particular by dendritic cells and/or macrophages, as compared to a negative control, in particular by at least 10%, more particularly by at least 20% as compared to the negative control.

In another aspect, the invention relates to the anti-CLEC-1A antibodies disclosed herein, or CLEC-1A-antagonist compounds, for use in the treatment of a disease or a deleterious condition, in particular wherein the phagocytosis exerted by dendritic cells and/or macrophages needs to be improved, and/or wherein the improvement of the phagocytosis capability of dendritic cells and/or macrophages treats the disease or the deleterious condition.

In another aspect, the invention relates to a specific anti-CLEC-1A compound as described above, for its use in the prevention and/or the treatment of a disease or a disorder in which the modulation of the phagocytosis capability by myeloid cells, in particular dendritic cells and/or macrophages, may improve the outcome of the disease or disorder, in particular by modulating the phagocytosis of tumor cells and/or secondary necrotic cells, wherein said anti-CLEC-1A compound is an antagonist of the interaction between human CLEC-1A and CLEC-1A ligand expressing cells, in particular CLEC-1A ligand-expressing tumor cells or cancer cells and/or secondary necrotic cells. Such antibodies can be identified using phagocytosis assay such as described in the examples of the present invention, including by flow cytometry or microscopy. In a more particular embodiment of the invention, said antibody or an antigen-binding fragment thereof is able to enhance the phagocytosis of cancer cells and/or secondary necrotic cells by myeloid cells, in particular by dendritic cells and/or macrophages, as compared to a negative control, in particular by at least 10%, more particularly by at least 20% as compared to the negative control. In a particular embodiment, the phagocytosis may be assessed according to the following experiment:

Macrophages (MΦ) are generated from monocytes with M-CSF (100 ng/mL) for 5 days;
Macrophages (MΦ) are then preincubated with the anti-CLEC1 compound for 2 hours and then cultured with the non-Hodgkin's lymphoma (Raji; CD20+) and the anti-CD20 mAb (Rituximab) respectively at 10 ng/mL providing the “Eat-me” signal, for 4 hours.
Phagocytosis analysis is performed by microscopy and the percentage of phagocytosis is calculated by the percentage of pHrodo (pHrodo-SE, Thermofisher) positive Raji cells in total Macrophages.

In another aspect, the invention relates to a specific anti-CLEC-1A compound as described above, for its use in the prevention and/or the treatment of a disease or a disorder in which T cells have deleterious effects, wherein said anti-CLEC-1A compound is an antagonist of the interaction between human CLEC-1A and secondary necrotic cells and/or tumor cells, and/or tumor cells present in a host having a cancer or developing a cancer and/or in the intracellular content of permeabilized secondary necrotic cells and/or in the intracellular content of permeabilized tumor cells.

In another aspect, the invention relates to a method of increasing the phagocytosis capability of myeloid cells, in particular of dendritic cells and/or macrophages, comprising the administration in a patient in need thereof of an effective amount of a a specific anti-CLEC-1A compound of the invention, in particular an anti-CLEC1A antibody or antigen-binding fragment thereof according to any embodiment disclosed herein; in particular said anti-CLEC-1A compound is administered simultaneously, separately or sequentially with a conventional treatment or with at least one second therapeutic agent as defined herein.

In another aspect, the invention relates to a specific anti-CLEC-1A compound as described above, for its use in the treatment of cancer in particular in the treatment of liquid or solid cancers, and more particularly in the treatment of lymphoma, colorectal cancer, mesothelioma or hepatocarcinoma.

In another aspect of the invention, it relates to a combination of therapeutic compounds comprising as a first therapeutic compound an CLEC-1A-antagonist compound, in particular an anti-CLEC-1A antibody or antigen-binding fragment as defined herein, and at least one second therapeutic compound selected from the group consisting of an anti-tumor targeting antibody, in particular an anti-tumor targeting antibody suitable for activating and/or enhancing the phagocytosis capability of macrophages, in particular M1 macrophages, or a chemotherapeutic agent. The present inventors have shown that such combinations are particularly suitable for treating cancers. As illustrated in the examples of the invention, these combinations exert a synergetic effect in the treatment of cancer, leading to a drastic reduction of tumor growth, tumor volume, and/or improve the survival rate.

### DETAILED DESCRIPTION OF THE INVENTION

The expression “secondary necrotic cells” or “cells under secondary necrosis” accordingly defines cells (including cell lines as disclosed herein) that have progressed toward stages of cellular changes characterized by hypercondensed chromatin (pyknosis), and nuclear fragmentation (karyorrhexis) and possibly the additional features of rupture of cytoplasmic membrane, release of activated caspase-3, further a possible cytoplasmic swelling and lysosomal membrane permeabilization. Cells under secondary necrosis are cells for which the apoptotic process proceeds to an autolytic necrotic outcome, i.e., an autolytic process of cell disintegration. The expression “secondary necrotic cells” or “cells under secondary necrosis” may similarly be properly defined by reference to markers of this specific stage in apoptotic cells wherein markers are known and used that may also enable to discriminate secondary necrotic cells from early apoptotic cells or from primary necrotic cells. Such markers include label-conjugated Annexin V and propidium iodide (PI): early-apoptotic cells are known to be Annexin V positive and PI negative (Annexin +/PI−) whereas late-apoptotic cells are known to be Annexin V positive and PI positive i.e. Annexin/PI double positive (Annexin +/PI+). These markers are sometimes used in the art to designate late-apoptotic cells. As used herein, permeabilized cells are cells in which is provided access to intracellular or intraorganellar antigens. Permeabilization allows entry through the cell membrane of antibodies, thereby allowing the binding into the intracellular content of these cells of the anti-CLEC1A compound of the invention with CLEC-1A expressed within the intracellular compartment of the cell but not on the cell membrane.

By “endogenous ligand”, it should be understood a ligand originating from the same species or within the same organism as the CLEC-1A receptor; e.g. an endogenous human CLEC-1A ligand is the human ligand(s) of human CLEC-1A receptor; an endogenous mice CLEC-1A ligand is the mice ligand(s) of mice CLEC-1A receptor.

As used herein, the term “antibody” refers to polyclonal antibodies, monoclonal antibodies or recombinant antibodies.

As used herein, a “monoclonal antibody” is intended to refer to a preparation of antibody molecules, antibodies that share a common heavy chain and common light chain amino acid sequence, in contrast with “polyclonal” antibody preparations that contain a mixture of antibodies of different amino acid sequence. Monoclonal antibodies can be generated by several known technologies like phage, bacteria, yeast or ribosomal display, as well as by classical methods exemplified by hybridoma-derived antibodies. Thus, the term “monoclonal” is used to refer to all antibodies derived from one nucleic acid clone.

The antibodies of the present invention include recombinant antibodies. As used herein, the term “recombinant antibody” refers to antibodies which are produced, expressed, generated or isolated by recombinant means, such as antibodies which are expressed using a recombinant expression vector transfected into a host cell; antibodies isolated from a recombinant combinatorial antibody library; antibodies isolated from an animal (e.g. a mouse) which is transgenic due to human immunoglobulin genes; or antibodies which are produced, expressed, generated or isolated in any other way in which particular immunoglobulin gene sequences (such as human immunoglobulin gene sequences) are assembled with other DNA sequences. Recombinant antibodies include, for example, chimeric and humanized antibodies.

As used herein, a “chimeric antibody” refers to an antibody in which the sequence of the variable domain derived from the germline of a mammalian species, such as a mouse, have been grafted onto the sequence of the constant domain derived from the germline of another mammalian species, such as a human.

As used herein, a “humanized antibody” refers to an antibody in which CDR sequences derived from the germline of another mammalian species, such as a mouse, have been grafted onto human framework sequences.

In an embodiment, the antibodies of the invention are humanized antibodies. In an embodiment, the antibodies of the invention are recombinant antibodies. In an embodiment, the antibodies of the invention are chimeric antibodies. In an embodiment, the antibodies of the invention are recombinant chimeric antibodies. In an embodiment, the antibodies of the invention are recombinant humanized antibodies. The antibodies of the invention may be de-immunized. By “de-immunized”, it should be understood that the antibody share a similar structure with the antibody of the invention, but the structure of the antibody is modified to lower the potential of unwanted T cell response by removing known epitope recognized by T cells in the structure of the antibody.

As used herein, an “antigen-binding fragment of an antibody” means a part of an antibody, i.e. a molecule corresponding to a portion of the structure of the antibody of the invention, that exhibits antigen-binding capacity for CLEC-1A, possibly in its native form; such fragment especially exhibits the same or substantially the same antigen-binding specificity for CLEC-1A compared to the antigen-binding specificity of the corresponding four-chain antibody. Advantageously, the antigen-binding fragments have a similar binding affinity as the corresponding 4-chain antibodies. However, antigen-binding fragment that have a reduced antigen-binding affinity with respect to corresponding 4-chain antibodies are also encompassed within the invention. The antigen-binding capacity can be determined by measuring the affinity between the antibody and the target fragment. These antigen-binding fragments may also be designated as “functional fragments” of antibodies.

As used herein, the term “CLEC-1” has its general meaning in the art and refers to C-type lectin-like receptor-1, particularly from a mammal species, more particularly a human CLEC-1. CLEC-1 belongs to the DECTIN-1 cluster of C type-lectin like receptors (CTLRs) including CLEC-2, DECTIN-1, CLEC-9A, MICL, MAH and LOX-1.

As used herein, the term “CLEC-1A” relates to a CLEC-1A from a mammal species, preferably a human CLEC-1A. A reference sequence of the human CLEC-1A corresponds to the sequence associated to the Accession number Q8NC01 Uniprot. Preferably, the term “human CLEC-1” or “human CLEC-1A” or “human CLEC-1 receptor” or “human CLEC-1A receptor” refers to the protein of amino acid sequence referenced by the Q8NC01 Uniprot accession number and encoded by CLEC1A gene referenced by the 51267 NCBI accession number. In the present description, the terms CLEC-1A, CLEC1A, CLEC1, CLEC-1, Clec1,Clec-1, Clec1A and Clec-1A are used interchangeably and all designate a CLEC1 receptor of a mammal corresponding to human CLEC-1A receptor corresponds to the sequence associated to the Accession number Q8NC01 Uniprot, an orthologue protein thereof, or a homologous protein thereof. In particular, CLEC-1A is, a protein having the amino acid sequence of SEQ ID No. 109. In particular, the extracellular domain of CLEC-1A is a protein having the amino acid sequence of SEQ ID No. 108.

As used herein, the term “CLEC-1 antagonist” has its general meaning in the art and refers to any compound, natural or synthetic, that blocks, suppresses, or reduces the biological activity of CLEC-1. In particular, the CLEC-1 antagonist inhibits the interactions between the CLEC-1 and at least one of its ligands. In particular, the CLEC-1 antagonist enhances T cells response, particularly increases T cells proliferation and/or cytokine synthesis such as IFNgamma. It may also refers to any compound, natural or synthetic, that blocks, suppresses, or reduces the biological activity of CLEC-1. In particular, the CLEC-1 antagonist inhibits the interactions between the receptor CLEC-1 and at least one of its ligands, more particularly all of its ligands. More particularly, a CLEC-1 antagonist can bind to receptor CLEC-1 or to any one of its ligands.

As used herein, “CLEC-1 antagonist” or “antagonist of CLEC-1” may correspond to a compound which binds to CLEC-1A and selected from the group of an antibody, an antigen-binding fragment of an antibody, an antigen-binding antibody mimetic, a macromolecule comprising an antigen-binding fragment of an antibody or a full antibody, small organic compounds, a protein, like but not limited to at least a fragment of the extra-cellular domain of CLEC-1A; or a functional equivalent of CLEC-1A, such a fragment may be combined with another molecule, like a peptide or a fragment of another protein like an antibody, which stabilized the structure of the a fragment of the extra-cellular domain of CLEC-1A leading to the provision of a fusion protein comprising at least a fragment of the extra-cellular domain of CLEC-1A. Such a fusion protein may for example comprise the fragment of the extra-cellular domain of CLEC-1A and a linker peptide, a tag, a Fc portion of an antibody.

The antagonist capability of an antibody may be assessed according to suitable experiments disclosed in the examples of the present invention, in particular in example 1 wherein antagonists of CLEC-1A according to the present invention have the capability to modulate, i.e. increase, the phagocytosis of tumor cells by myeloid cells. In particular, an antibody or antigen-binding fragment thereof may be considered as an antagonist of CLEC-1A, in particular of human CLEC-1A, when (i) it reduces the binding of the extra-cellular domain of CLEC-1A, in particular when it reduces the binding of a fusion protein comprising the extracellular domain of human CLEC-1A receptor fused with a Fc fragment of a human immunoglobulin, in particular a human IgG, to secondary necrotic cells and/or tumor cells and/or to the intracellular content of secondary necrotic cells, particularly to permeabilized RAJI cells and/or to apoptotic PBMCs as compared to the same binding experiment in absence of the antagonist antibody candidate; and (ii) it increases the phagocytosis of tumor cells by myeloid cells as compared to the same experiment in absence of the antagonist compound. A binding reduction is considered when the binding is reduced by at least 1-log, more particularly at least 2-log and most preferably at least 3-log as compared to the negative experiment. An increase in the phagocytosis of tumor cells is considered when the phagocytosis is raised by at least 10%, preferably at least 20%; and most preferably at least 30%.

The antibody and antigen-binding fragment of the invention may be defined according to structural features. Antigen-binding fragments of antibodies are fragments which comprise their hypervariable domains designated CDRs (Complementary Determining Regions) or part(s) thereof encompassing the recognition site for the antigen, i.e. the extracellular domain of CLEC-1A.

Each Light and Heavy chain variable domains (respectively VL and VH) of a four-chain immunoglobulin has three CDRs, designated VL-CDR1 (or LCDR1), VL-CDR2 (or LCDR2), VL-CDR3 (or LCDR3) and VH-CDR1 (or HCDR1), VH-CDR2 (or HCDR2), VH-CDR3 (or HCDR3), respectively.

The skilled person is able to determine the location of the various regions/domains of antibodies by reference to the standard definitions in this respect set forth, including a reference numbering system, a reference to the numbering system of KABAT or by application of the IMGT “collier de perle” algorithm. In this respect, for the definition of the sequences of the invention, it is noted that the delimitation of the regions/domains may vary from one reference system to another. Accordingly, the regions/domains as defined in the present invention encompass sequences showing variations in length or localization of the concerned sequences within the full-length sequence of the variable domains of the antibodies, of approximately +/-10%.

In a particular embodiment of the invention, the CDR domains of the antibodies are designated according to the Kabat nomenclature. In another particular embodiment of the invention, the CDR domains of the antibodies are designated according to the IMGT nomenclature. In other words, any or all CDR domain of the antibodies or the antigen-binding fragment thereof of the invention may be defined by Kabat nomenclature; any or all CDR domain of the antibodies or the antigen-binding fragment thereof of the invention may be defined by IMGT nomenclature. More particularly, all CDR domains of the antibodies or the antigen-binding fragment thereof of the invention are defined by the Kabat nomenclature.

Based on the structure of four-chain immunoglobulins, antigen-binding fragments can thus be defined by comparison with sequences of antibodies in the available databases and prior art, and especially by comparison of the location of the functional domains in these sequences, noting that the positions of the framework and constant domains are well defined for various classes of antibodies, especially for IgGs, in particular for mammalian IgGs. Such comparison also involves data relating to 3-dimensional structures of antibodies.

For illustration purpose of specific embodiments of the invention, antigen binding fragments of an antibody that contain the variable domains comprising the CDRs of said antibody encompass Fv, dsFv, scFv, Fab, Fab′, F(ab′)2. Fv fragments consist of the VL and VH domains of an antibody associated together by hydrophobic interactions; in dsFv fragments, the VH:VL heterodimer is stabilized by a disulphide bond; in scFv fragments, the VL and VH domains are connected to one another via a flexible peptide linker thus forming a single-chain protein. Fab fragments are monomeric fragments obtainable by papain digestion of an antibody; they comprise the entire L chain, and a VH-CH1 fragment of the H chain, bound together through a disulfide bond. The F(ab′)2 fragment can be produced by pepsin digestion of an antibody below the hinge disulfide; it comprises two Fab′ fragments, and additionally a portion of the hinge region of the immunoglobulin molecule. The Fab′ fragments are obtainable from F(ab′)2 fragments by cutting a disulfide bond in the hinge region. F(ab′)2 fragments are divalent, i.e. they comprise two antigen binding sites, like the native immunoglobulin molecule; on the other hand, Fv (a VH:VL dimmer constituting the variable part of Fab), dsFv, scFv, Fab, and Fab′ fragments are monovalent, i.e. they comprise a single antigen-binding site. These basic antigen-binding fragments of the invention can be combined together to obtain multivalent antigen-binding fragments, such as diabodies, tribodies or tetrabodies. These multivalent antigen-binding fragments are also part of the present invention.

As used herein, the term “bispecific” antibodies refer to antibodies that recognize two different antigens by virtue of possessing at least one region (e.g. derived from a variable region of a first antibody) that is specific for a first antigen, and at least a second region (e.g. derived from a variable region of a second antibody) that is specific for a second antigen. A bispecific antibody specifically binds to two target antigens and is thus one type of multispecific antibody. Multispecific antibodies, which recognize two or more different antigens, can be produced by recombinant DNA methods or include, but are not limited to, antibodies produced chemically by any convenient method. Bispecific antibodies include all antibodies or conjugates of antibodies, or polymeric forms of antibodies which are capable of recognizing two different antigens. Bispecific antibodies include antibodies that have been reduced and reformed so as to retain their bivalent characteristics and to antibodies that have been chemically coupled so that they can have several antigen recognition sites for each antigen such as BiME (Bispecific Macrophage Enhancing antibodies), BiTE (bispecific T cell engager), DART (Dual affinity retargeting); DNL (dock-and-lock), DVD-Ig (dual variable domain immunoglobulins).

All the embodiments disclosed herein for antibodies are transposed mutatis mutandis to any compound according to the invention, in particular to antigen-binding antibody fragments, humanized antibodies and chimeric antibodies and recombinant antibodies.

In the following description of the invention, the term anti-CLEC-1A compound means either an antibody, an antigen-binding fragment, whether humanized or not, whether chimeric or not, whether recombinant or not, or a macromolecule comprising such an antibody or antigen-binding fragment thereof. When the term anti-CLEC-1A antibody is used, the same compounds are encompassed by this term, except when specified in relation to a particular embodiment of the invention.

A “specific anti-CLEC-1A antibody” is a compound that exhibits specific binding for CLEC-1A and which does not exhibit specific binding for another compound, binding being in each case detectable by methods known in the art like but not limited to Biacore analysis, Blitz analysis, ELISA assay or Scatchard plot. A specific “anti-CLEC-1A compound” may nonetheless cross-react with another compound than CLEC-1A, the notion of specificity does not exclude that an antibody may cross-react with other polypeptides than CLEC-1A, but with a lower affinity. Hence, specific anti-CLEC-1A compound may also be defined as an antibody that exhibits high binding affinity for CLEC-1A but that nevertheless exhibit low binding affinity for another compound.

### Antibodies and Antigen-Binding Fragments Thereof

In a first aspect, it is disclosed an antibody or antigen-binding fragment thereof that specifically binds to the extracellular domain of human C-type lectin-like receptor-1 member A receptor (CLEC-1A receptor) which comprises:
VHCDR1 of SEQ ID No: 57; SEQ ID No: 65; SEQ ID No: 73; SEQ ID No: 81; SEQ ID No: 89 or SEQ ID No: 97; in particular SEQ ID No: 65, SEQ ID No: 81 or SEQ ID No: 97; and
VHCDR2 of SEQ ID No: 59; SEQ ID No: 67; SEQ ID No: 75; SEQ ID No: 83; or SEQ ID No: 91; in particular SEQ ID No. 67; SEQ ID No: 75 or SEQ ID No: 83; and
VHCDR3 of SEQ ID No: 61; SEQ ID No: 69; SEQ ID No: 77; SEQ ID No: 85 or SEQ ID No: 93; in particular SEQ ID No. 69; SEQ ID No: 77 or SEQ ID No: 85; and
an antibody light chain variable domain comprising three VLCDRs wherein their amino acid sequence is selected from:
VLCDR1 of SEQ ID No: 4; SEQ ID No: 12; SEQ ID No: 20; SEQ ID No: 28 or SEQ ID No: 36; in particular SEQ ID No. 12; SEQ ID No: 20 or SEQ ID No: 28; and
VLCDR2 of SEQ ID No: 6; SEQ ID No: 14; SEQ ID No: 22; SEQ ID No: 30 or SEQ ID No: 38; in particular SEQ ID No. 14; SEQ ID No: 22; or SEQ ID No: 30 and
VLCDR3 of SEQ ID No: 8; SEQ ID No: 16; SEQ ID No: 24; SEQ ID No: 32 or SEQ ID No: 40; in particular SEQ ID No. 16; SEQ ID No: 24 or SEQ ID No: 32.

In another embodiment of the invention, it is disclosed an antibody or antigen-binding fragment thereof that specifically binds to the extracellular domain of human C-type lectin-like receptor-1 member A receptor (CLEC-1A receptor) which comprises:
an antibody heavy chain variable domain comprising three VHCDRs wherein their amino acid sequences are respectively selected from:
   VHCDR1 of SEQ ID No: 57; SEQ ID No: 65; SEQ ID No: 73; SEQ ID No: 81; SEQ ID No: 89; SEQ ID No: 97 or SEQ ID No: 103; in particular SEQ ID No: 73, SEQ ID No: 81 or SEQ ID No: 97; and
   VHCDR2 of SEQ ID No: 59; SEQ ID No: 67; SEQ ID No: 75; SEQ ID No: 83; SEQ ID No: 91 or SEQ ID No: 105; in particular SEQ ID No: 75 or SEQ ID No: 83; and
   VHCDR3 of SEQ ID No: 61; SEQ ID No: 69; SEQ ID No: 77; SEQ ID No: 85; SEQ ID No: 93 or ; SEQ ID No: 107; in particular SEQ ID No: or SEQ ID No: 85; and
an antibody light chain variable domain comprising three VLCDRs wherein their amino acid sequence is selected from:
   VLCDR1 of SEQ ID No: 4; SEQ ID No: 12; SEQ ID No: 20; SEQ ID No: 28; SEQ ID No: 36 or SEQ ID No: 49; in particular SEQ ID No: 20 or SEQ ID No: 28; and
   VLCDR2 of SEQ ID No: 6; SEQ ID No: 14; SEQ ID No: 22; SEQ ID No: 30; SEQ ID No: 38 or SEQ ID No: 51; in particular SEQ ID No: 22; or SEQ ID No: 30 and
   VLCDR3 of SEQ ID No: 8; SEQ ID No: 16; SEQ ID No: 24; SEQ ID No: 32; SEQ ID No: 40 or SEQ ID No: 53 in particular SEQ ID No: 24 or SEQ ID No: 32.

In a particular embodiment of the invention, the antibody or antigen-binding fragment thereof comprises the following CDR domains:
A VHCDR1 comprising or consisting of the amino acid sequence set forth in SEQ ID No: 65, SEQ ID No: 81 or SEQ ID No: 97; and
A VHCDR2 comprising or consisting of the amino acid sequence set forth in SEQ ID NO. 67 or of SEQ ID No: 75 or of SEQ ID No: 83; and
A VHCDR3 comprising or consisting of the amino acid sequence set forth in SEQ ID NO. 69 or of SEQ ID No: 77 or of SEQ ID No: 85; and
A VLCDR1 comprising or consisting of the amino acid sequence set forth in SEQ ID NO. 12 or of SEQ ID No: 20 or of SEQ ID No: 28; and
A VLCDR2 comprising or consisting of the amino acid sequence set forth in SEQ ID NO. 14 or of SEQ ID No: 22 or of SEQ ID No: 30; and
A VLCDR3 comprising or consisting of the amino acid sequence set forth in SEQ ID NO. 16 or of SEQ ID No: 24 or of SEQ ID No: 32.

In a particular embodiment of the invention, the antibody or antigen-binding fragment thereof comprises the following CDR domains:
A VHCDR1 comprising or consisting of the amino acid sequence set forth in SEQ ID No: 73, SEQ ID No: 81 or SEQ ID No: 97; and
A VHCDR2 comprising or consisting of the amino acid sequence set forth in SEQ ID No: 75 or of SEQ ID No: 83; and
A VHCDR3 comprising or consisting of the amino acid sequence set forth in SEQ ID No: 77 or of SEQ ID No: 85; and
A VLCDR1 comprising or consisting of the amino acid sequence set forth in SEQ ID No: 20 or of SEQ ID No: 28; and
A VLCDR2 comprising or consisting of the amino acid sequence set forth in SEQ ID No: 22 or of SEQ ID No: 30; and
A VLCDR3 comprising or consisting of the amino acid sequence set forth in SEQ ID No: 24 or of SEQ ID No: 32.

Antibodies according to this embodiment are particularly suitable for enhancing the phagocytosis of tumor cells by dendritic cells. Antibodies according to this particular definition have an affinity for human CLEC-1A which is suitable for use in therapy and have at the same time a better effect at the same concentration on the phagocytosis capability of tumor cells by dendritic cells as compared to other anti-CLEC-1A antibody, in particular as compared to the control anti-CLEC-1A antibody used in the examples of the invention (see FIGS. 1-3).

In a particular embodiment, the antibody heavy chain variable domain comprises the VHCDR1, VHCDR2 and VHCDR3 of sequence:
SEQ ID No: 57; SEQ ID No: 59 and SEQ ID No: 61 respectively; or
SEQ ID No: 65; SEQ ID No: 67 and SEQ ID No: 69 respectively; or
SEQ ID No: 73; SEQ ID No: 75 and SEQ ID No: 77 respectively; or
SEQ ID No: 81; SEQ ID No: 83 and SEQ ID No: 85 respectively; or
SEQ ID No: 89; SEQ ID No: 91 and SEQ ID No: 93 respectively; or
SEQ ID No: 97; SEQ ID No: 75 and SEQ ID No: 77 respectively.
   These combinations of CDR domains correspond respectively to the CDR domains present on the heavy chain of exemplified antibodies 15E3, 11H11, 5D1, 6C5 10F4 and 14H9 respectively.

In a particular embodiment, the antibody light chain variable domain comprises the VLCDR1, VLCDR2 and VLCDR3 of sequence:
SEQ ID No: 4; SEQ ID No: 6 and SEQ ID No: 8 respectively; or
SEQ ID No: 12; SEQ ID No: 14 and SEQ ID No: 16 respectively; or
SEQ ID No: 20; SEQ ID No: 22 and SEQ ID No: 24 respectively; or
SEQ ID No: 28; SEQ ID No: 30 and SEQ ID No: 32 respectively; or
SEQ ID No: 36; SEQ ID No: 38 and SEQ ID No: 40 respectively.
   These combinations of CDR domains correspond respectively to the CDR domains present on the light chain of exemplified antibodies 15E3, 11H11, 5D1 and 14H9 (both share the same CDR VLCDRs), 6C5 and 10F4 respectively.

In a particular embodiment of the invention, the antibody heavy chain variable domain comprises the VHCDR1, VHCDR2 and VHCDR3 of sequence SEQ ID No: 97; SEQ ID No: 75 and SEQ ID No: 77 respectively, and the antibody light chain variable domain comprises the VLCDR1, VLCDR2 and VLCDR3 of sequence SEQ ID No: 20; SEQ ID No: 22 and SEQ ID No: 24 respectively. Antibodies according to this definition may be particularly suitable for modulating, in particular, enhancing the phagocytosis of tumor cells and/or secondary necrotic cells by myeloid cells, in particular by dendritic cells or macrophages, in particular in vitro and/or in vivo.

In a particular embodiment of the invention, the antibody heavy chain variable domain comprises the VHCDR1, VHCDR2 and VHCDR3 of sequence SEQ ID No: 81; SEQ ID No: 83 and SEQ ID No: 85 respectively, and the antibody light chain variable domain comprises the VLCDR1, VLCDR2 and VLCDR3 of sequence SEQ ID No: 28; SEQ ID No: 30 and SEQ ID No: 32 respectively. Antibodies according to this definition may be particularly suitable for modulating, in particular, enhancing the phagocytosis of tumor cells and/or secondary necrotic cells by myeloid cells, in particular by dendritic cells or macrophages, in particular in vitro and/or in vivo.

In a particular embodiment of the invention, the antibody heavy chain variable domain comprises the VHCDR1, VHCDR2 and VHCDR3 of sequence SEQ ID No: 65; SEQ ID No: 67 and SEQ ID No: 69 respectively, and the antibody light chain variable domain comprises the VLCDR1, VLCDR2 and VLCDR3 of sequence SEQ ID No: 12; SEQ ID No: 14 and SEQ ID No: 16 respectively. Antibodies according to this definition may be particularly suitable for modulating, in particular, enhancing the phagocytosis of tumor cells and/or secondary necrotic cells by myeloid cells, in particular by dendritic cells or macrophages, in particular in vitro and/or in vivo.

In a particular embodiment of the invention, the antibody heavy chain variable domain comprises the VHCDR1, VHCDR2 and VHCDR3 of sequence SEQ ID No: 89; SEQ ID No: 91 and SEQ ID No: 93 respectively, and the antibody light chain variable domain comprises the VLCDR1, VLCDR2 and VLCDR3 of sequence SEQ ID No: 36; SEQ ID No: 38 and SEQ ID No: 40 respectively. Antibodies according to this definition may be particularly suitable for modulating, in particular, enhancing the phagocytosis of tumor cells and/or secondary necrotic cells by myeloid cells, in particular by dendritic cells or macrophages, in particular in vitro and/or in vivo.

In a particular embodiment of the invention, the antibody heavy chain variable domain comprises the VHCDR1, VHCDR2 and VHCDR3 of sequence SEQ ID No: 73; SEQ ID No: 75 and SEQ ID No: 77 respectively, and the antibody light chain variable domain comprises the VLCDR1, VLCDR2 and VLCDR3 of sequence SEQ ID No: 20; SEQ ID No: 22 and SEQ ID No: 24 respectively. Antibodies according to this definition may be particularly suitable for modulating, in particular, enhancing the phagocytosis of tumor cells and/or secondary necrotic cells by myeloid cells, in particular by dendritic cells or macrophages, in particular in vitro and/or in vivo.

In another particular embodiment, the antibody heavy chain variable domain comprises or consists of the amino acid sequence set forth in SEQ ID No: 55; SEQ ID No: 63; SEQ ID No: 71; SEQ ID No: 79; SEQ ID No: 87 or SEQ ID No: 95. These heavy chain variable domains correspond respectively to the heavy variable domains of exemplified antibodies 15E3, 11H11, 5D1, 6C5, 10F4 and 14H9.

In another particular embodiment, the antibody light chain variable domain comprises or consists of the amino acid sequence set forth in SEQ ID No: 2; SEQ ID No: 10; SEQ ID No: 18; SEQ ID No: 26; SEQ ID No: 34 or SEQ ID No: 42. These light chain variable domains correspond respectively to the light variable domains of exemplified antibodies 15E3, 11H11, 5D1, 6C5, 10F4 and 14H9.

In another particular embodiment, an antibody or an antigen-binding fragment thereof of the invention comprises:
a heavy variable domain comprising or consisting of the amino acid sequence set forth in SEQ ID No: 55 and a light variable domain comprising or consisting of the amino acid sequence set forth in SEQ ID No: 2; or
a heavy variable domain comprising or consisting of the amino acid sequence set forth in SEQ ID No: 63 and a light variable domain comprising or consisting of the amino acid sequence set forth in SEQ ID No: 10; or
a heavy variable domain comprising or consisting of the amino acid sequence set forth in SEQ ID No: 71 and a light variable domain comprising or consisting of the amino acid sequence set forth in SEQ ID No: 18; or
a heavy variable domain comprising or consisting of the amino acid sequence set forth in SEQ ID No: 79 and a light variable domain comprising or consisting of the amino acid sequence set forth in SEQ ID No: 26; or
a heavy variable domain comprising or consisting of the amino acid sequence set forth in SEQ ID No: 87 and a light variable domain comprising or consisting of the amino acid sequence set forth in SEQ ID No: 34; or
a heavy variable domain comprising or consisting of the amino acid sequence set forth in SEQ ID No: 95 and a light variable domain comprising or consisting of the amino acid sequence set forth in SEQ ID No: 42.
   These combinations of a heavy variable domain with a light variable domain correspond to respectively to the heavy and light variable domains of exemplified antibodies 15E3, 11H11, 5D1, 6C5, 10F4, 14H9 and 21B1.

In another particular embodiment, an antibody or an antigen-binding fragment thereof of the invention comprises a heavy variable domain comprising or consisting of the amino acid sequence set forth in SEQ ID No: 95 and a light variable domain comprising or consisting of the amino acid sequence set forth in SEQ ID No: 42; which correspond respectively to the heavy and variable domains of exemplified antibody 14H9. Antibodies according to this definition may be particularly suitable for modulating, in particular, enhancing the phagocytosis of tumor cells and/or secondary necrotic cells by myeloid cells, in particular by dendritic cells or macrophages, in particular in vitro and/or in vivo.

In another particular embodiment, an antibody or an antigen-binding fragment thereof of the invention comprises a heavy variable domain comprising or consisting of the amino acid sequence set forth in SEQ ID No: 79 and a light variable domain comprising or consisting of the amino acid sequence set forth in SEQ ID No: 26; which correspond respectively to the heavy and variable domains of exemplified antibody 6C5. Antibodies according to this definition may be particularly suitable for modulating, in particular, enhancing the phagocytosis of tumor cells and/or secondary necrotic cells by myeloid cells, in particular by dendritic cells or macrophages, in particular in vitro and/or in vivo.

In another particular embodiment, an antibody or an antigen-binding fragment thereof of the invention comprises a heavy variable domain comprising or consisting of the amino acid sequence set forth in SEQ ID No: 63 and a light variable domain comprising or consisting of the amino acid sequence set forth in SEQ ID No: 10; which correspond respectively to the heavy and variable domains of exemplified antibody 11H11. Antibodies according to this definition may be particularly suitable for modulating, in particular, enhancing the phagocytosis of tumor cells and/or secondary necrotic cells by myeloid cells, in particular by dendritic cells or macrophages, in particular in vitro and/or in vivo.

In another particular embodiment, an antibody or an antigen-binding fragment thereof of the invention comprises a heavy variable domain comprising or consisting of the amino acid sequence set forth in SEQ ID No: 87 and a light variable domain comprising or consisting of the amino acid sequence set forth in SEQ ID No: 34; which correspond respectively to the heavy and variable domains of exemplified antibody 10F4. Antibodies according to this definition may be particularly suitable for modulating, in particular, enhancing the phagocytosis of tumor cells and/or secondary necrotic cells by myeloid cells, in particular by dendritic cells or macrophages, in particular in vitro and/or in vivo.

In another particular embodiment, an antibody or an antigen-binding fragment thereof of the invention comprises a heavy variable domain comprising or consisting of the amino acid sequence set forth in SEQ ID No: 71 and a light variable domain comprising or consisting of the amino acid sequence set forth in SEQ ID No: 18; which correspond respectively to the heavy and variable domains of exemplified antibody 5D1. Antibodies according to this definition may be particularly suitable for modulating, in particular, enhancing the phagocytosis of tumor cells and/or secondary necrotic cells by myeloid cells, in particular by dendritic cells or macrophages, in particular in vitro and/or in vivo.

The various antibody molecules and fragments may derive from any of the commonly known immunoglobulin classes (isotypes), including but not limited to IgA, secretory IgA, IgE, IgG and IgM. IgG subclasses are also well known to those in the art and include but are not limited to human IgGl, IgG2, IgG3 and IgG4. In a particular embodiment of the invention, the variable regions of the antibody may be associated with antibody constant regions, like IGgl, IgG2, IgG3 or IgG4 constant regions. These constant regions may be further mutated or modified, by methods known in the art, for modifying their binding capability towards Fc receptor. In a particular embodiment, the antibody or antigen-binding fragment thereof according to the invention is a humanized monoclonal antibody, in particular wherein the antibody light chain constant domain is derived from a human kappa light chain constant domain, in particular wherein the light chain constant domain comprises or consists of the sequence of SEQ ID No: 112, for example encoded by the nucleotide sequence of SEQ ID No: 111, and wherein the antibody heavy chain constant domain is derived from a human IgG1, IgG2, IgG3, or IgG4 heavy chain constant domain, in particular wherein the antibody heavy chain constant domain comprises or consists of the amino acid sequence of SEQ ID No: 113 (human Fc IgG1), SEQ ID No: 114 (human Fc IgG2) SEQ ID No: 115 (human Fc IgG4); SEQ ID No: 116 (mouse FcG1) or SEQ ID No: 117 (mouse FcG4—for example encoded by the nucleotide sequence of SEQ Id No: 118).

In another embodiment, the antibody or antigen-binding fragment thereof binds to an human CLEC-1A with an affinity of at least about 1×10-6 M, 1×10-7 M,1×10-8 M, 1×10-9 M, 1×10-10 M, 1×10-11 M, 1×10-12 M, or more, and/or bind to a target with an affinity that is at least two-fold greater than its affinity for another compound than human CLEC-1A receptor., In a particular embodiment, the antibody or antigen-binding fragment of the invention binds to human CLEC-1A with an affinity constant (1(D) of at least than 1E-07 M, more particularly of at least 1E-08 M. In a particular embodiment, the antibody or antigen-binding fragment thereof binds to a human CLEC-1A with an affinity over 1-log, more particularly over 2-log, and most preferably over 3-log, as compared to the binding of control anti-CLEC-1A antibody to CLEC-la in the same binding conditions. The binding experiment may be proceeded according to any one the binding experiment disclosed in the examples of the invention.

In a particular embodiment, an anti-CLEC-1A compound is CLEC-1A specific when the effective dose of the compound to reach 50% of the maximum signal (ED50) according to the invention has an ED50 value for human CLEC-1A lower than 1500 ng/ml The ED50 may be determined according to methods known in the art, or by the method disclosed in the examples of the present invention, like cytometry illustrated on FIG. 4. In a particular embodiment, the binding between an anti-CLEC-1A antibody and human CLEC-1A as defined here above may be considered specific when the effective dose of the compound to reach 50% of the maximum signal (EC50) in a binding assay is lower than 1200 ng/ml, more particularly lower than 800 ng/ml, and still more particularly lower than 400 ng/ml. Such an ability may for example be assessed according to the methods illustrated in the examples of the present invention.

In another particular embodiment, a specific anti-CLEC-1A compound according to the invention has an ED50 value (also referenced EC 50 value) for human CLEC-1A comprised between 1 ng/ml and 1000 ng/ml, more particularly between 5 ng/ml and 1500 ng/ml, more particularly 800 ng/ml. The EC50 may be determined according to methods known in the art, or by the method disclosed in the examples of the present invention, for example according to the method disclosed in relation to the data illustrated on FIG. 4 and issued from example 2.

The term “ED50” and as used herein refers to the measure of the effectiveness of a compound (e.g., an anti-CLEC-1A compound) in eliciting a biological or biochemical function (e.g., the function or activity of CLEC-1A) by 50%. For example, EC50 indicates how much of an anti-CLEC-1A compound is needed to elicit the activity of CLEC-1A by half. That is, it is the half maximal (50%) effective concentration of an anti-CLEC-1A compound (50% ED, or ED50). ED50 represents the concentration of a drug that is required for 50% effectiveness in vitro. The ED50 can be determined by techniques known in the art, for example, by constructing a dose-response curve and examining the effect of different concentrations of the anti-CLEC-1A compound on CLEC-1A binding to Fc-CLEC. A method is for example disclosed in the examples of the present invention.

In the invention, it can also be considered that an anti-CLEC-1A compound is an antagonist of CLEC-1A if said compound induces an increase superior to 1 log, preferably superior to 2 log, more preferably superior to 3 log, most preferably superior to 4 log, of the KD value of Fc-CLEC-1A protein to CLEC-1A in a binding competitive assay wherein the antagonist antibody is present. This experiment may be conducted according to Blitz method or ELISA, for example in the experimental conditions illustrated in the examples of the invention.

An antibody or an antigen-binding fragment thereof, which is a humanized antibody can also be derived by substitution of amino acid residue(s) present in constant region(s) of variable chains (VH and/or VL), for human amino acid residue(s) having corresponding location in human antibodies according to standard definition and numbering, wherein the substitution level is from 1% to 80%, more preferably from 1% to 50%, still more preferably form 1% to 20%, in particular from 1% to 18% of the residues in said framework regions. Said constant regions include those of framework regions (FRs) defined in four-chain antibodies identified in particular by reference to KABAT numbering.

Anti-CLEC-1A antibodies may be humanized according to known methods. As examples, the different combinations of CDRs disclosed herein may be grafted on human heavy chain variable domain and/or light chain variable domain. The chimeric, humanized and/or de-immunized antibodies of the invention can belong to any class of immunoglobulins, like the non-modified antibodies. Preferably, they belong to a subclass of the IgG class such as IgG1, IgG2, IgG3 or IgG4.

Methods for preparing recombinant antibodies (or antigen-binding fragment thereof), or chimeric antibodies by combining the variable regions of an antibody with appropriate linkers, or with the constant regions of another antibody, are well known in the art.

Also encompassed by the present invention is an antibody or an antigen-binding fragment thereof, in particular a chimeric or a humanized antibody or antigen-binding fragment thereof, which competes with an antibody comprising the amino acid sequence of SEQ ID No. 71 as its variable heavy domain and the amino acid sequence of SEQ ID No: 18 as its light variable domain, in particular which is the chimeric antibody 5D1 illustrated in the examples of the present invention, for binding to a CLEC-1A receptor and which antagonizes CLEC-1A binding to its target.

In particular, the antibody or an antigen-binding fragment thereof of the invention specifically binds to the extracellular domain of human C-type lectin-like receptor-1 member A receptor (CLEC-1A receptor), and further competes with an antibody comprising or consisting of a heavy variable domain comprising or consisting of SEQ ID No. 71 and a light variable domain comprising or consisting of SEQ ID No. 18, in particular comprising or consisting of a heavy domain comprising or consisting of SEQ ID No. 121 and a light domain comprising or consisting of SEQ ID No. 128, for binding to a human CLEC-1A receptor, and is an antagonist of human CLEC-1A, in particular antagonizes the binding of human CLEC-1A, particularly the binding of the extra-cellular domain of human CLEC-1A, to at least one of its ligand (in particular its target), particularly expressed by secondary necrotic cells and/or tumor cells. In particular, the antibody or an antigen-binding fragment thereof of the invention specifically binds to the extracellular domain of human C-type lectin-like receptor-1 member A receptor (CLEC-1A receptor), and further competes with an antibody comprising or consisting of a heavy variable domain comprising or consisting of SEQ ID No. 63 and a light variable domain comprising or consisting of SEQ ID No. 10, in particular comprising or consisting of a heavy domain comprising or consisting of SEQ ID No. 120 and a light domain comprising or consisting of SEQ ID No. 127, for binding to a human CLEC-1A receptor, and is an antagonist of human CLEC-1A, in particular antagonizes the binding of human CLEC-1A, particularly the binding of the extra-cellular domain of human CLEC-1A, to at least one of its ligand (in particular its target), particularly expressed by secondary necrotic cells and/or tumor cells.
In a more particular embodiment of the invention, said antibody or an antigen-binding fragment thereof is also able to enhance the phagocytosis of cancer cells and/or secondary necrotic cells by myeloid cells, in particular by dendritic cells and/or macrophages, as compared to a negative control, in particular by at least 10%, more particularly by at least 20% as compared to the negative control.. Particularly, said antibody or an antigen-binding fragment thereof correlates when used in vivo and/or in vitro with a modulation, in particular an increase, of the phagocytosis of tumor cells and/or secondary necrotic cells by myeloid cells, in particular by dendritic cells and/or macrophages, as compared to a negative control, in particular the phagocytosis of tumor cells and/or secondary necrotic cells is increased by at least 10%, more particularly by at least 20% as compared to the negative control.

Cross-competing antibodies (or compounds) and antibodies (or compounds) that recognize the CLEC-1A receptor can be identified using routine techniques such as an immunoassay, for example, by showing the ability of one antibody to block the binding of another antibody to a target antigen, e.g. , a competitive binding assay. Competitive binding may be determined using an assay such as described in the examples of the present invention. In particular, competitive binding may be determined using the method illustrated in example 10, wherein antibodies interaction and competition on His-CLEC1 is studied by ELISA. Cross-competition is present if the tested anti-CLEC-1A compound reduces binding of the other antibody by at least by 50%, at least by 60%, specifically at least by 70% and more specifically at least by 80% and vice versa in comparison to the positive control which lacks one of said antibodies (or compounds).

### Antagonist Compounds Exhibiting Functional Features and Specifically Binding to CLEC-1A or Comprising a Particular Portion of CLEC-1A.

The invention also concerns an antagonist compound of CLEC-1A which:
(i) correlates with a modulation, in particular an enhancement, of the phagocytosis of tumor cells by myeloid cells, in particular by dendritic cells and/or macrophage (more particularly human dendritic cells and/or human macrophages); and
(ii) correlates with a modulation, in particular with an enhancement, of the proliferation of T cells, in particular human T cells; and/or
(iii) correlates with a modulation, in particular with an enhancement, of the activation of T cells, in particular human T cell; and
   wherein the antagonist compound is selected from the group consisting of polypeptides, peptides, antibodies, antigen-binding fragments thereof, antigen-binding antibody mimetics, functional equivalent of CLEC-1A, in particular of human CLEC-1A, or an organic molecule, and
   wherein the antagonist compound binds to the extracellular domain of human C-type lectin-like receptor-1 member A receptor (“CLEC-1A” or “CLEC-1A receptor”) and competes with an antibody comprising or consisting of a heavy variable domain comprising or consisting of SEQ ID No. 63 and a light variable domain comprising or consisting of SEQ ID No. 10, in particular comprising or consisting of a heavy domain comprising or consisting of SEQ ID No. 120 and a light domain comprising or consisting of SEQ ID No. 127, for binding to a human CLEC-1A receptor, and is an antagonist of human CLEC-1, in particular antagonizes the binding of human CLEC-1A, particularly the binding of the extra-cellular domain of human CLEC-1A, to at least one of its ligand (in particular its target), particularly expressed by secondary necrotic cells and/or tumor cells.
   The antagonist compound is in particular antibody or an antigen-binding fragment, or an antigen-binding antibody mimetic.

In a particular embodiment, the antagonist compound of CLEC-1A:
(i) correlates with a modulation, in particular an enhancement, of the phagocytosis of tumor cells by myeloid cells, in particular by dendritic cells and/or macrophage (more particularly human dendritic cells and/or human macrophages); and
(ii) correlates with a modulation, in particular with an enhancement, of the proliferation of T cells, in particular human T cells; and/or
(iii) correlates with a modulation, in particular with an enhancement, of the activation of T cells, in particular human T cell; and
   wherein the antagonist compound is selected from the group consisting of polypeptides, peptides, antibodies, antigen-binding fragments thereof, antigen-binding antibody mimetics, functional equivalent of CLEC-1A, in particular of human CLEC-1A, or an organic molecule, and
   wherein the antagonist compound binds to the extracellular domain of human C-type lectin-like receptor-1 member A receptor (“CLEC-1A” or “CLEC-1A receptor”) and competes with an antibody comprising or consisting of a heavy variable domain comprising or consisting of SEQ ID No. 71 and a light variable domain comprising or consisting of SEQ ID No. 18, in particular comprising or consisting of a heavy domain comprising or consisting of SEQ ID No. 121 and a light domain comprising or consisting of SEQ ID No. 128, for binding to a human CLEC-1A receptor, and is an antagonist of human CLEC-1, in particular antagonizes the binding of human CLEC-1A, particularly the binding of the extra-cellular domain of human CLEC-1A, to at least one of its ligand (in particular its target), particularly expressed by secondary necrotic cells and/or tumor cells.
   The antagonist compound is in particular antibody or an antigen-binding fragment, or an antigen-binding antibody mimetic.

The antagonist compound is in particular selected from the group of an antibody, an antigen-binding fragment of an antibody, including chimeric, humanized, fully humanized antibodies and fragments thereof, including de-immunized antibodies and antigen-binding fragments thereof, antigen-binding antibody mimetic, a macromolecule comprising an antigen-binding fragment of an antibody or a full antibody, small organic compounds, a protein, like but not limited to a protein or a peptide comprising at least a fragment of the extra-cellular domain of CLEC-1A; or a functional equivalent of CLEC-1A, such a fragment may be combined with another molecule, like a peptide or a fragment of another protein like an antibody, which stabilized the structure of the a fragment of the extra-cellular domain of CLEC-1A receptor leading to the provision of a fusion protein comprising at least a fragment of the extra-cellular domain of CLEC-1A. Such a fusion protein may for example comprise a fragment of the extra-cellular domain of CLEC-la receptor and a linker peptide, a tag, a Fc portion of an antibody. In a particular embodiment, the fusion protein may comprise a fragment of at least 10 contiguous, amino acid residues of the extracellular domain of CLEC-1A of SEQ ID No. 108, in particular the fusion protein may comprise a fragment of a CLEC protein comprising at least 70%, preferably at least 80%, more preferably at least 90%, of the extracellular domain of CLEC-1, in particular a fragment of a CLEC1 protein comprising or consisting of at least 70%, preferably at least 80%, more preferably at least 90%, consecutive amino acids in the sequence set forth in SEQ ID No. 108 (EC-CLEC1), in particular comprising the amino acid sequence of SEQ ID No. 108. In a particular embodiment of the invention, the fusion protein may comprise a portion having an identity of at least 90% with a portion of at least 10 contiguous amino acid residues of SEQ ID No. 108. In a particular embodiment, the functional equivalent of CLEC-1A is Fc-CLEC-1A as exemplified in the present description, more particularly FC-CLEC-1A of SEQ ID No. 110. In a particular embodiment, the agonist compound is any antagonist anti-CLEC-1A antibody as disclosed here above. In a particular embodiment, the agonist compound is an anti-CLEC-1A antibody comprising a heavy chain variable domain and a light chain variable domain, the heavy chain variable domain being selected from the group consisting of amino acid sequence of SEQ ID No. 95 or of SEQ ID No. 79 or of SEQ ID No. 63, and the light chain variable domain being selected from the group consisting of the amino acid sequence of SEQ ID No. 42 or of SEQ ID No. 26 or of SEQ ID No. 10.

The invention also concerns genetic constructs encoding at least a portion of the specific anti-CLEC-1A receptor compounds described therein.

To this end, the invention also relates to nucleic acid molecule(s) encoding a compound according to any one of the definitions of the compound disclosed herein. In other words, the nucleic acid molecule(s) encode(s) at least the 6 CDR domains of an antibody or antigen-binding fragment thereof. Accordingly, nucleic acid molecules according to the invention may be chosen from:
SEQ ID No: 1, SEQ ID No: 3, SEQ ID No: 5, SEQ ID No: 7, SEQ ID No: 9, SEQ ID No: 11, SEQ ID No: 13, SEQ ID No: 15, SEQ ID No: 17, SEQ ID No: 19, SEQ ID No: 21, SEQ ID No: 23, SEQ ID No: 25, SEQ ID No: 27, SEQ ID No: 29, SEQ ID No: 31, SEQ ID No: 33, SEQ ID No: 35, SEQ ID No: 37, SEQ ID No: 39, SEQ ID No: 41, SEQ ID No: 43, SEQ ID No: 44, SEQ ID No: 45, SEQ ID No: 46, SEQ ID No: 48, SEQ ID No: 50, SEQ ID No: 52, SEQ ID No: 54, SEQ ID No: 56, SEQ ID No: 58, SEQ ID No: 60, SEQ ID No: 62, SEQ ID No: 64, SEQ ID No: 66, SEQ ID No: 68, SEQ ID No: 70, SEQ ID No: 72, SEQ ID No: 74, SEQ ID No: 76, SEQ ID No: 78, SEQ ID No: 80, SEQ ID No: 82, SEQ ID No: 84, SEQ ID No: 86, SEQ ID No: 88, SEQ ID No: 90, SEQ ID No: 92, SEQ ID No: 94, SEQ ID No: 96, SEQ ID No: 98, SEQ ID No: 99, SEQ ID No: 100, SEQ ID No: 102, SEQ ID No: 104 and/or SEQ ID No: 106; with the proviso that the nucleic acid molecule(s) encode(s) at least the 6 CDR domains of an antibody or antigen-binding fragment thereof according to any embodiment disclosed herein;
and more particularly chosen from the SEQ ID No: 1, SEQ ID No: 3, SEQ ID No: 5, SEQ ID No: 7, SEQ ID No: 9, SEQ ID No: 11, SEQ ID No: 13, SEQ ID No: 15, SEQ ID No: 17, SEQ ID No: 19, SEQ ID No: 21, SEQ ID No: 23, SEQ ID No: 25, SEQ ID No: 27, SEQ ID No: 29, SEQ ID No: 31, SEQ ID No: 33, SEQ ID No: 35, SEQ ID No: 37, SEQ ID No: 39, SEQ ID No: 41, SEQ ID No: 43, SEQ ID No: 44, SEQ ID No: 45,SEQ ID No: 54, SEQ ID No: 56, SEQ ID No: 58, SEQ ID No: 60, SEQ ID No: 62, SEQ ID No: 64, SEQ ID No: 66, SEQ ID No: 68, SEQ ID No: 70, SEQ ID No: 72, SEQ ID No: 74, SEQ ID No: 76, SEQ ID No: 78, SEQ ID No: 80, SEQ ID No: 82, SEQ ID No: 84, SEQ ID No: 86, SEQ ID No: 88, SEQ ID No: 90, SEQ ID No: 92, SEQ ID No: 94, SEQ ID No: 96, SEQ ID No: 98 and/or SEQ ID No: 99, with the proviso that the nucleic acid molecule(s) encode(s) at least the 6 CDR domains of an antibody or antigen-binding fragment thereof according to any embodiment disclosed herein.
   The invention may also relate to a combination of a first nucleic acid molecule and a second nucleic acid molecule. A first nucleic acid molecule encoding a variable heavy chain domain may be selected from the group consisting of: SEQ ID No. 54, SEQ ID No: 56, SEQ ID No: 58, SEQ ID No: 60, SEQ ID No: 62, SEQ ID No: 64, SEQ ID No: 66, SEQ ID No: 68, SEQ ID No: 70, SEQ ID No: 72, SEQ ID No: 74, SEQ ID No: 76, SEQ ID No: 78, SEQ ID No: 80, SEQ ID No: 82, SEQ ID No: 84, SEQ ID No: 86, SEQ ID No: 88, SEQ ID No: 90, SEQ ID No: 92, SEQ ID No: 94, SEQ ID No: 96, SEQ ID No: 98, SEQ ID No: 99, SEQ ID No: 100, SEQ ID No: 102, SEQ ID No: 104 and/or SEQ ID No: 106, more particularly selected from the group consisting of SEQ ID No. 54, SEQ ID No: 56, SEQ ID No: 58, SEQ ID No: 60, SEQ ID No: 62, SEQ ID No: 64, SEQ ID No: 66, SEQ ID No: 68, SEQ ID No: 70, SEQ ID No: 72, SEQ ID No: 74, SEQ ID No: 76, SEQ ID No: 78, SEQ ID No: 80, SEQ ID No: 82, SEQ ID No: 84, SEQ ID No: 86, SEQ ID No: 88, SEQ ID No: 90, SEQ ID No: 92, SEQ ID No: 94, SEQ ID No: 96, SEQ ID No: 98 and/or SEQ ID No: 99.

A second nucleic acid molecule encoding a variable light chain domain may be selected from the group consisting of : SEQ ID No: 1, SEQ ID No: 3, SEQ ID No: 5, SEQ ID No: 7, SEQ ID No: 9, SEQ ID No: 11, SEQ ID No: 13, SEQ ID No: 15, SEQ ID No: 17, SEQ ID No: 19, SEQ ID No: 21, SEQ ID No: 23, SEQ ID No: 25, SEQ ID No: 27, SEQ ID No: 29, SEQ ID No: 31, SEQ ID No: 33, SEQ ID No: 35, SEQ ID No: 37, SEQ ID No: 39, SEQ ID No: 41, SEQ ID No: 43, SEQ ID No: 44, SEQ ID No: 45, SEQ ID No: 46, SEQ ID No: 48, SEQ ID No: 50 and SEQ ID No: 52, more particularly selected from the group consisting of SEQ ID No: 1, SEQ ID No: 3, SEQ ID No: 5, SEQ ID No: 7, SEQ ID No: 9, SEQ ID No: 11, SEQ ID No: 13, SEQ ID No: 15, SEQ ID No: 17, SEQ ID No: 19, SEQ ID No: 21, SEQ ID No: 23, SEQ ID No: 25, SEQ ID No: 27, SEQ ID No: 29, SEQ ID No: 31, SEQ ID No: 33, SEQ ID No: 35, SEQ ID No: 37, SEQ ID No: 39, SEQ ID No: 41, SEQ ID No: 43, SEQ ID No: 44 and/or SEQ ID No: 45.

The combination of a first and a second nucleic acid molecule encode(s) at least the 6 CDR domains of the antibody or antigen-binding fragment thereof according to any embodiment disclosed herein.

Such nucleic acid molecules may be inserted within an expression vector, like a plasmid for example, suitable for expression of the encoded sequence within a host cell.

### Combinations of Compounds.

The invention also concerns combination of compounds comprising a first therapeutic agent and at least one second therapeutic agent.

In a preferred embodiment, the first therapeutic agent is an antibody or antigen-binding fragment thereof according to any embodiment disclosed herein. In another embodiment, the first therapeutic compound may also be a CLEC-1A antagonist as defined herein, like but not limited to a compound which binds to CLEC-1A and selected from the group of an antibody, an antigen-binding fragment of an antibody, an antigen-binding antibody mimetic, a macromolecule comprising an antigen-binding fragment of an antibody or a full antibody, a small organic compound, a protein, like but not limited to at least a fragment of the extra-cellular domain of CLEC-1A, in particular the extra-cellular domain of CLEC-1A or, a Fc-CLEC-1 protein as defined herein and corresponding to the amino acid sequence of SEQ ID No: 110.The at least one second therapeutic agent is selected from the list consisting of a chemotherapeutic agent, tumor-targeting antibody including anti-hCD20-hIgG1, anti-hEGFR-hIgG1, anti-hHER2-hIgG1 or antigen-binding fragment thereof, in particular a tumor-targeting monoclonal antibody or antigen-binding fragment thereof, more particularly a tumor-targeting monoclonal antibody or antigen-binding fragment thereof which activates and/or enhances the phagocytosis capability of macrophages, and still more particularly a monoclonal antibody selected from the group consisting of alemtuzumab, atezolizumab, bevacizumab, anti-hEGFR-hIgG1 monoclonal tumor-targeting antibody such as cetuximab, herceptin, panitumumab, anti-hCD20-hIgG1 monoclonal tumor-targeting antibody such as rituximab, anti-hHER2-hIgG1 monoclonal tumor-targeting trastuzumab, an anti-PDL-1 antibody, and an anti-CD47 antibody, or another antibody or monoclonal antibody selected from the group consisting of an anti-PD1 antibody and an anti-SIRPa antibody; and/or a chemotherapeutic agent, in particular a cytotoxic agent with anti-proliferative, pro-apoptotic, cell cycle arresting and/or differentiation inducing effect, more particularly a cytotoxic agent selected from the group consisting of cytotoxic antibody, alkylating drugs, anthracyclines, antimetabolites, anti-microtubule agents, topoisomerase inhibitors, alkaloids, bleomycin, antineoplastic drugs, cyclophosphamide.

A tumor-targeting antibody may be defined as a of therapeutic monoclonal antibody that recognizes tumor-specific membrane proteins, block cell signalling, and induce tumor killing through Fc-driven innate immune responses.

In a particular embodiment of the invention, the first therapeutic agent is an antibody defined by its CDR domains as disclosed herein, and the second therapeutic agent is Rituximab, or another antibody or monoclonal antibody selected from the group consisting of an anti-PD1 antibody, an anti-PDL-1 antibody, an anti-CD47 antibody, and an anti-SIRPa antibody. In a particular embodiment, the first therapeutic agent is an antagonist compound selected from the group consisting of:
an antibody or an antigen-binding fragment thereof, which specifically binds to the extracellular domain of human C-type lectin-like receptor-1 member A receptor (CLEC-1A receptor) and which competes with an antibody comprising or consisting of a heavy variable domain comprising or consisting of SEQ ID No. 71 and a light variable domain comprising or consisting of SEQ ID No. 18, in particular comprising or consisting of a heavy domain comprising or consisting of SEQ ID No. 121 and a light domain comprising or consisting of SEQ ID No. 128, for binding to a human CLEC-1A receptor, and which is an antagonist of human CLEC-1; and
an antibody or an antigen-binding fragment thereof, which specifically binds to the extracellular domain of human C-type lectin-like receptor-1 member A receptor (CLEC-1A receptor) and, which competes with an antibody comprising or consisting of a heavy variable domain comprising or consisting of SEQ ID No. 63 and a light variable domain comprising or consisting of SEQ ID No. 10, in particular comprising or consisting of a heavy domain comprising or consisting of SEQ ID No. 120 and a light domain comprising or consisting of SEQ ID No. 127, for binding to a human CLEC-1A receptor, and which is an antagonist of human CLEC-1; and
an antibody or an antigen-binding fragment thereof, which specifically binds to the extracellular domain of human C-type lectin-like receptor-1 member A receptor (CLEC-1A receptor) and which correlates when used in vivo and/or in vitro with a modulation, in particular an increase, of the phagocytosis of tumor cells and/or secondary necrotic cells by myeloid cells, in particular by dendritic cells and/or macrophages, as compared to a negative control, in particular by at least 10% as compared to the negative control; and
   the second therapeutic agent being selected from the list defined here above or here below.

The chemotherapeutic agent may be a conventional cytotoxic agent, i.e. a compound that induces irreversible lethal lesions through interference with DNA replication, mitosis, etc. following exposure. These agents may have anti-proliferative, pro-apoptotic, cell cycle arresting, and differentiation inducing effects. These agents are preferentially selected from the group consisting of alkylating drugs (cisplatin, chlorambucil, procarbazine, carmustine), anthracyclines and other cytotoxic antibiotics, antimetabolites (i.e. methotrexate, cytarabine, gemcitabine), anti-microtubule agents (i.e. vinblastine, paclitaxel, docetaxel), topoisomerase inhibitors (i.e. etoposide, doxorubicin), alkaloids (i.e. Vincristine, Vinblastine, Vinorelbine, Camptothecin) or bleomycin (inhibiting incorporation of thymidine into DNA strands).

The inventors shown that the use in combination of an antagonist of CLEC-1A, and in particular an antibody antagonist of CLEC-1A, in combination with another therapeutic agent, in particular rituximab, enhances the phagocytosis capability of macrophages, in particular M1 macrophages, and anti-CLEC-1A antagonist compounds are therefore suitable for enhancing the therapeutic effect of a simultaneously, separately, or sequentially administered second therapeutic agent.

In another embodiment of the invention, it is provided a combination of compounds comprising a first therapeutic agent which comprises at least a fragment of CLEC-1A protein or a functional equivalent of CLEC1 and at least one second therapeutic agent, wherein:

the fist therapeutic agent may be a fragment of a CLEC-1A protein as defined herein, in particular a fragment of a CLEC-1A protein comprising at least 70%, preferably at least 80%, more preferably at least 90%, of the extracellular domain of CLEC-1A, in particular a fragment of a CLEC1A protein comprising or consisting of at least 70%, preferably at least 80%, more preferably at least 90%, consecutive amino acids in the sequence set forth in SEQ ID No. 108 (EC-CLEC1), in particular comprising the amino acid sequence of SEQ ID No. 108, or a fusion protein comprising a fragment of the CLEC-1A protein as defined here in, or a Fc-CLEC1 protein comprising or consisting of the amino acid sequence set forth in SEQ ID No: 110, possibly encoded by the nucleotide sequence of SEQ ID No. 133; or a functional equivalent of CLEC-1A as defined here below and
The second therapeutic agent is selected from the list consisting of a tumor-targeting antibody or antigen-binding fragment thereof, in particular a tumor-targeting monoclonal antibody or antigen-binding fragment thereof, more particularly a tumor-targeting monoclonal antibody or antigen-binding fragment thereof which activates and/or enhances the phagocytosis capability of macrophages, and still more particularly a monoclonal antibody selected from the group consisting of alemtuzumab, atezolizumab, bevacizumab, cetuximab, herceptin, panitumumab, rituximab, trastuzumab, an anti-PDL-1 antibody, and an anti-CD47 antibody, or another antibody or monoclonal antibody selected from the group consisting of an anti-PD1 antibody and an anti-SIRPa antibody.

Functional equivalents of CLEC-1 include but are not limited to molecules that bind to a ligand of CLEC-1 and comprise all or a portion of the extracellular domains of CLEC-1 so as to form a soluble receptor that is capable to trap the ligand of CLEC-1. Thus, the functional equivalents include soluble forms of CLEC-1. A suitable soluble form of these proteins, or functional equivalents thereof, might comprise, for example, a truncated form of the protein from which the transmembrane domain has been removed by chemical, proteolytic or recombinant methods. Particularly, the functional equivalent consisting of a sequence having at least 80% identity, more particularly at least 85%, at least 86%, at least 87%, at least 88%, at least 89%, at least 90%, at least 91%, at least 92%, at least 93%, at least 94%, at least 95%, at least 96%, at least 97%, at least 98% and even more particularly at least 99% of identity with the corresponding protein over the entire length of the corresponding protein. As used herein, the term “corresponding protein” refers to the protein for which the functional equivalent of the invention has similar function. The percentages of identity to which reference is made in the presentation of the present invention are determined on the basis of a global alignment of sequences to be compared, that is to say, on an alignment of sequences over their entire length, using for example the algorithm of Needleman and Wunsch 1970. This sequence comparison can be done for example using the needle software by using the parameter “Gap open” equal to 10.0, the parameter “Gap Extend” equal to 0.5, and a matrix “BLOSUM 62”. Software such as needle is available on the website ebi.ac.uk worldwide, under the name “needle”. The term “a functionally equivalent fragment” as used herein also may mean any fragment or assembly of fragments of CLEC-1 that binds to a ligand of CLEC-1. Accordingly, the present invention provides a polypeptide, in particular a functional equivalent, capable of inhibiting binding of CLEC-1 to at least one ligand of CLEC-1, which polypeptide comprises consecutive amino acids having a sequence which corresponds to the sequence of at least a portion of an extracellular domain of CLEC-1, which portion binds to a ligand of CLEC-1. In some embodiments, the polypeptide, in particular the functional equivalent, corresponds to an extracellular domain of CLEC-1.

In some embodiments, the functional equivalent of CLEC-1 is fused to a heterologous polypeptide to form a fusion protein. As used herein, a “fusion protein” comprises all or part (typically biologically active) of a functional equivalent of the present invention operably linked to a heterologous polypeptide (i.e., a polypeptide other than the same polypeptide). Within the fusion protein, the term “operably linked” is intended to indicate that the functional equivalent of the present invention and the heterologous polypeptide are fused in-frame to each other. The heterologous polypeptide can be fused to the N-terminus or C-terminus of the functional equivalent of the present invention.

In some embodiments, the functional equivalent of CLEC-1 is fused to an immunoglobulin constant domain (Fc region) to form an immunoadhesin. Immunoadhesins can possess many of the valuable chemical and biological properties of human antibodies. Since immunoadhesins can be constructed from a human protein sequence with a desired specificity linked to an appropriate human immunoglobulin hinge and constant domain (Fc) sequence, the binding specificity of interest can be achieved using entirely human components. Such immunoadhesins are minimally immunogenic to the patient, and are safe for chronic or repeated use. In some embodiments, the Fc region is a native sequence Fc region. In some embodiments, the Fc region is a variant Fc region. In still another embodiment, the Fc region is a functional Fc region. As used herein, the term “Fc region” is used to define a C-terminal region of an immunoglobulin heavy chain, including native sequence Fc regions and variant Fc regions. Although the boundaries of the Fc region of an immunoglobulin heavy chain might vary, the human IgG heavy chain Fc region is usually defined to stretch from an amino acid residue at position Cys226, or from Pro230, to the carboxyl-terminus thereof. The adhesion portion and the immunoglobulin sequence portion of the immunoadhesin may be linked by a minimal linker. The immunoglobulin sequence typically, but not necessarily, is an immunoglobulin constant domain. The immunoglobulin moiety in the chimeras of the present invention may be obtained from IgG1, IgG2, IgG3 or IgG4 subtypes, IgA, IgE, IgD or IgM, but typically IgG1 or IgG4. In some embodiments, the functional equivalent ofCLEC-1 and the immunoglobulin sequence portion of the immunoadhesin are linked by a minimal linker. As used herein, the term “linker” refers to a sequence of at least one amino acid that links the polypeptide of the invention and the immunoglobulin sequence portion. Such a linker may be useful to prevent steric hindrances. In some embodiments, the linker has 4; 5; 6; 7; 8; 9; 10; 11; 12; 13; 14; 15; 16; 17; 18; 19; 20; 21; 22; 23; 24; 25; 26; 27; 28; 29; 30 amino acid residues. However, the upper limit is not critical but is chosen for reasons of convenience regarding e.g. biopharmaceutical production of such polypeptides. The linker sequence may be a naturally occurring sequence or a non-naturally occurring sequence. If used for therapeutical purposes, the linker is typically non-immunogenic in the subject to which the immunoadhesin is administered. One useful group of linker sequences are linkers derived from the hinge region of heavy chain antibodies as described in WO 96/34103 and WO 94/04678. Other examples are poly-alanine linker sequences.

The polypeptides of the invention may be produced by any suitable means, as will be apparent to those of skill in the art. In order to produce sufficient amounts of polypeptides for use in accordance with the present invention, expression may conveniently be achieved by culturing under appropriate conditions recombinant host cells containing the polypeptide of the invention. In particular, the polypeptide is produced by recombinant means, by expression from an encoding nucleic acid molecule. Systems for cloning and expression of a polypeptide in a variety of different host cells are well known. When expressed in recombinant form, the polypeptide is in particular generated by expression from an encoding nucleic acid in a host cell. Any host cell may be used, depending upon the individual requirements of a particular system. Suitable host cells include bacteria mammalian cells, plant cells, yeast and baculovirus systems. Mammalian cell lines available in the art for expression of a heterologous polypeptide include Chinese hamster ovary cells. HeLa cells, baby hamster kidney cells and many others. Bacteria are also preferred hosts for the production of recombinant protein, due to the ease with which bacteria may be manipulated and grown. A common, preferred bacterial host is E coli.

The polypeptides of the invention, fragments thereof and fusion proteins according to the invention can exhibit post-translational modifications, including, but not limited to glycosylations, (e.g., N-linked or 0-linked glycosylations), myristylations, palmitylations, acetylations and phosphorylations (e.g., serine/threonine or tyrosine)

In a particular embodiment, the combination of compounds comprising a first therapeutic agent and at least one second therapeutic agent comprises:

a Fc-CLEC1 protein comprising or consisting of the amino acid sequence set forth in SEQ ID No: 110, possibly encoded by the nucleotide sequence of SEQ ID No. 133, or a functional equivalent thereof and
a monoclonal antibody selected from the group consisting of alemtuzumab, atezolizumab, bevacizumab, cetuximab, herceptin, panitumumab, rituximab, trastuzumab, in particular rituximab.

In a particular embodiment, the therapeutic agents may be administered simultaneously, separately, or sequentially in the treatment of a disease.

The invention also concerns a pharmaceutical composition comprising a first therapeutic agent as defined herein, in particular, more particularly a CLEC-1A antagonist which is an antibody or antigen-binding fragment thereof according to any embodiment disclosed herein, either alone or in combination with a second therapeutic agent, with a pharmaceutical suitable vehicle, which are pharmaceutically acceptable for a formulation capable of being administered to a patient in need thereof. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions.

The invention also concerns a pharmaceutical composition comprising a first therapeutic agent as defined herein, in particular, more particularly a CLEC-1A antagonist which is an antibody or antigen-binding fragment thereof according to any embodiment disclosed herein, either alone or in combination with a second therapeutic agent, and/or with a pharmaceutical suitable vehicle as defined here in, for use in a combination therapy with another treatment including the use of a medicament comprising a chemotherapeutic agent, a radiotherapy agent, an immunotherapeutic agent (such as a tumor-targeting monoclonal antibody), a cell therapy agents (such as CAR-T cells), an immunosuppressive agent, a pro-apoptotic agent, an antibiotic , a targeted cancer therapy, and/or a probiotic, in particular for simultaneous, separated, or sequential administration to a patient in need thereof.

The invention also concerns a method of treating cancer in a human subject in need thereof comprising administering to the subject a therapeutically effective amount of a first therapeutic agent as defined herein, in particular a CLEC-1A antagonist which is an antibody or antigen-binding fragment thereof according to any embodiment disclosed herein; wherein said first therapeutic agent is used in combination with a conventional treatment.

As used herein, the term “standard or conventional treatment” refers to any treatment of cancer (drug, radiotherapy, etc) usually administrated to a subject who suffers from cancer.

In particular, the first therapeutic agent is used in combination with a chemotherapeutic agent, a radiotherapy agent, an immunotherapeutic agent (such as a tumor-targeting monoclonal antibody), a cell therapy agent (such as CAR-T cells), an immunosuppressive agent, a pro-apoptotic agent, an antibiotic, a targeted cancer therapy, and/or a probiotic.

The present invention also concerns the use of the anti-CLEC1A antibodies and antigen-binding fragment disclosed herein, and antagonist compounds as disclosed herein, for use in the treatment of a cancer. The terms “cancer” has its general meaning in the art and refers to a group of diseases involving abnormal cell growth with the potential to invade or spread to other parts of the body. The term “cancer” further encompasses both primary and metastatic cancers. Examples of cancers that may treated by methods and compositions of the invention include, but are not limited to, cancer from the bladder, blood, bone, bone marrow, brain, breast, colon, oesophagus, gastrointestinal, gum, head, kidney, liver, lung, nasopharynx, neck, ovary, prostate, skin, stomach, testis, tongue, or uterus. In addition, the cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; non encapsulating sclerosing carcinoma; adrenal cortical carcinoma; endometroid carcinoma; skin appendage carcinoma; apocrine adenocarcinoma; sebaceous adenocarcinoma; ceruminous; adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; Paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; and roblastoma, malignant; Sertoli cell carcinoma; leydig cell tumor, malignant; lipid cell tumor, malignant; paraganglioma, malignant; extra-mammary paraganglioma, malignant; pheochromocytoma; glomangio sarcoma; malignant melanoma; amelanotic melanoma; superficial spreading melanoma; malign melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brennertumor, malignant; phyllodestumor, malignant; synovial sarcoma; mesothelioma, malignant; dysgerminoma; embryonal carcinoma; teratoma, malignant; strumaovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblasticodontosarcoma; ameloblastoma, malignant; ameloblasticfibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; Hodgkin's disease; Hodgkin's lymphoma; paragranuloma; malignant lymphoma, small lymphocytic; malignant lymphoma, large cell, diffuse; malignant lymphoma, follicular; mycosis fungoides; other specified non-Hodgkin's lymphomas; malignant histiocytosis; multiple myeloma; mast cell sarcoma; immunoproliferative small intestinal disease; leukaemia; lymphoid leukaemia; plasma cell leukaemia; erythroleukemia; lymphosarcoma cell leukaemia; myeloid leukaemia; basophilic leukaemia; eosinophilic leukaemia; monocyticleukaemia; mast cell leukaemia; megakaryoblasticleukaemia; myeloid sarcoma; and hairy cell leukaemia.

In some embodiments, the subject suffers from a cancer selected from the group consisting of bile duct cancer, bladder cancer, bone cancer, brain and central nervous system cancer, breast cancer, Castleman disease cervical cancer, colorectal cancer, endometrial cancer, oesophagus cancer, gallbladder cancer, gastrointestinal carcinoid tumors, Hodgkin's disease, non-Hodgkin's lymphoma, Kaposi's sarcoma, kidney cancer, laryngeal and hypopharyngeal cancer, liver cancer, lung cancer, mesothelioma, plasmacytoma, nasal cavity and paranasal sinus cancer, nasopharyngeal cancer, neuroblastoma, oral cavity and oropharyngeal cancer, ovarian cancer, pancreatic cancer, penile cancer, pituitary cancer, prostate cancer, retinoblastoma, rhabdomyosarcoma, salivary gland cancer, skin cancer, stomach cancer, testicular cancer, thymus cancer, thyroid cancer, vaginal cancer, vulvar cancer, and uterine cancer.

The present invention also concerns the use of the anti-CLEC1A antibodies and antigen-binding fragment disclosed herein, and antagonist compounds as disclosed herein, for use in the treatment, including the preventive treatment, of a deleterious condition or a disease, in particular wherein the phagocytosis capability of myeloid cells, in particular of dendritic cells and/or macrophages, is involved. In a particular embodiment, the disease or condition is selected from the group consisting of cancer, in particular a cancer as listed here above, more particularly liquid cancers, solid cancers, lymphoma, colorectal cancers, mesothelioma or hepatocarcinoma.

The present invention also concerns the use of the anti-CLEC1A antibodies and antigen-binding fragment disclosed herein, and antagonist compounds as disclosed herein, for use in the treatment, including the preventive treatment, of a deleterious condition or a disease, in particular wherein the stimulation of the phagocytosis capability of dendritic cells may improve or treat the condition or the disease. In a particular embodiment, the disease or condition is selected from the group consisting of cancer, in particular a cancer as listed here above, more particularly liquid cancers, solid cancers, lymphoma, colorectal cancers, mesothelioma or hepatocarcinoma.

The present invention also concerns the use of the anti-CLEC1A antibodies and antigen-binding fragment disclosed herein, and antagonist compounds as disclosed herein, for use in the treatment, including the preventive treatment, of any disease or condition susceptible of being improved or prevented by increasing the phagocytosis capability of myeloid cells, in particular of dendritic cells and/or macrophages. In particular, the disease or condition is selected from the group consisting of cancer, in particular a cancer as listed here above, more particularly liquid cancers, solid cancers, lymphoma, colorectal cancers, mesothelioma or hepatocarcinoma.

The present invention also concerns the use of the anti-CLEC1A antibodies and antigen-binding fragment disclosed herein, and antagonist compounds as disclosed herein, for use in the treatment, including the preventive treatment, of a deleterious condition or a disease, in particular wherein T cells are involved, and wherein the proliferation of T cells is involved. In a particular embodiment, the disease or condition is selected from the group consisting of cancer, in particular a cancer as listed here above, more particularly liquid cancers, solid cancers, lymphoma, colorectal cancers, mesothelioma or hepatocarcinoma.

The present invention also concerns a method of increasing the phagocytosis capability of myeloid cells, in particular of dendritic cells and/or macrophages, comprising the administration in a patient in need thereof of an effective amount of a first therapeutic agent as defined herein, in particular an anti-CLEC1A antibody or antigen-binding fragment thereof according to any embodiment disclosed herein; in particular said first therapeutic agent is administered simultaneously, separately or sequentially with a conventional treatment or with at least one second therapeutic agent as defined herein.

The present invention also concerns the use of a first therapeutic agent as defined herein, in particular an anti-CLEC-1A antagonist compound according to any definition disclosed herein, more particularly an anti-CLEC1A antibody or antigen-binding fragment thereof according to any embodiment disclosed herein for the manufacture of a medicament. In particular, the present invention concerns the use of such an anti-CLEC-1A antagonist compound for use in the manufacture of a medicament for treating and/or preventing cancer, in particular a cancer as listed here above, more particularly liquid cancers, solid cancers, lymphoma, colorectal cancers, mesothelioma or hepatocarcinoma.

The present invention also concerns a method for treating or preventing a disease by administering to a patient in need thereof a therapeutic amount of a first therapeutic agent as defined herein, in particular an anti-CLEC-1A antagonist compound according to any definition disclosed herein, more particularly an anti-CLEC1A antibody or antigen-binding fragment thereof according to any embodiment disclosed herein. In particular, the present invention concerns a method for treating or preventing a cancer, in particular a cancer as listed here above, more particularly liquid cancers, solid cancers, lymphoma, colorectal cancers, mesothelioma or hepatocarcinoma.

The invention also concerns uses of the compounds, compositions, and combinations of compounds as defined herein, in particular uses for preventing or treating a disease or a disorder. Accordingly, it is provided an antagonist compound of the invention, in particular an antibody or antigen-binding fragment thereof of the invention, or the nucleic acid molecule or a combination of nucleic acid molecules according to the invention, or a combination of compounds according the invention, for use in the prevention and/or the treatment of a disease or a disorder, in particular a human disease or a human disorder, in which the increase of the phagocytosis capability by myeloid cells, in particular dendritic cells and/or macrophages, improves or prevents the disease or disorder.

It is also provided an antagonist compound of the invention, in particular an antibody or antigen-binding fragment thereof of the invention, or the nucleic acid molecule or a combination of nucleic acid molecules according to the invention, or a combination of compounds according the invention, for use in a treatment of a disease or a condition wherein induction of phagocytosis in a patient improves or prevents the disease or condition.
It is also provided an antagonist compound of the invention, in particular an antibody or antigen-binding fragment thereof of the invention, or the nucleic acid molecule or a combination of nucleic acid molecules according to the invention, or a combination of compounds according the invention, for the treatment of a patient having a cancer, in particular a liquid or a solid cancer, more particularly a lymphoma, a colorectal cancer, a mesothelioma or a hepatocarcinoma, an inflammatory disease, a chronic infection or sepsis.
It is also provided an antagonist compound of the invention, in particular an antibody or antigen-binding fragment thereof of the invention, or the nucleic acid molecule or a combination of nucleic acid molecules according to the invention, or a combination of compounds according the invention, for use in a combination therapy, wherein a first medicament comprising a chemotherapeutic agent, a radiotherapy agent, an immunotherapeutic agent (such as a tumor-targeting monoclonal antibody), a cell therapy agents (such as CAR-T cells), an immunosuppressive agent, a pro-apoptotic agent, an antibiotic, a targeted cancer therapy, and/or a probiotic, in particular for simultaneous, separated, or sequential administration, is administered to a patient in need thereof.
In a particular embodiment of the recited uses, the antagonist compound of the invention may be selected from the group consisting of:
an antibody or an antigen-binding fragment thereof, which specifically binds to the extracellular domain of human C-type lectin-like receptor-1 member A receptor (CLEC-1A receptor) and which competes with an antibody comprising or consisting of a heavy variable domain comprising or consisting of SEQ ID No. 71 and a light variable domain comprising or consisting of SEQ ID No. 18, in particular comprising or consisting of a heavy domain comprising or consisting of SEQ ID No. 121 and a light domain comprising or consisting of SEQ ID No. 128, for binding to a human CLEC-1A receptor, and which is an antagonist of human CLEC-1; and
an antibody or an antigen-binding fragment thereof, which specifically binds to the extracellular domain of human C-type lectin-like receptor-1 member A receptor CLEC-1A receptor) and, which competes with an antibody comprising or consisting of a heavy variable domain comprising or consisting of SEQ ID No. 63 and a light variable domain comprising or consisting of SEQ ID No. 10, in particular comprising or consisting of a heavy domain comprising or consisting of SEQ ID No. 120 and a light domain comprising or consisting of SEQ ID No. 127, for binding to a human CLEC-1A receptor, and which is an antagonist of human CLEC-1; and
an antibody or an antigen-binding fragment thereof, which specifically binds to the extracellular domain of human C-type lectin-like receptor-1 member A receptor (CLEC-1A receptor) and which correlates when used in vivo and/or in vitro with a modulation, in particular an increase, of the phagocytosis of tumor cells and/or secondary necrotic cells by myeloid cells, in particular by dendritic cells and/or macrophages, as compared to a negative control, in particular by at least 10% as compared to the negative control.

TABLE 1
Sequences of the specific CDR domain
of the heavy variable domain of anti-
bodies according to the invention
(according to the Kabat system)

| | | SEQ ID |
|---|---|---|
| CDRs | Sequences | No: |
| 15E3chim_VH wt_CDR1 | DYVIA | 57 |
| 15E3chim_VH wt_CDR2 | EIYPGSGSIYYNEKFKG | 59 |
| 15E3chim_VH wt_CDR3 | STVVAFDY | 61 |
| 11H11chim_VH wt_CDR2 | WINTNTGEPTYADDFKG | 67 |
| 11H11chim_VH wt_CDR3 | GAPAWFTY | 69 |
| 5D1chim_VH wt_CDR1 | SYGVH | 73 |
| 5D1chim_VH wt_CDR2 | VIWSDGSTIYNSALKS | 75 |
| 14H9chim_VH wt_CDR2 | VIWSDGSTIYNSALKS | 75 |
| 5D1chim_VH wt_CDR3 | HGGYYNYFDY | 77 |
| 14H9chim_VH wt_CDR3 | HGGYYNYFDY | 77 |
| 6C5chim_VH wt_CDR1 | DYVIS | 81 |
| 6C5chim_VH wt_CDR2 | EIYPGSGNTYYNEKFKG | 83 |
| 6C5chim_VH wt_CDR3 | GGSSHFDY | 85 |
| 10F4chim_VH wt_CDR1 | DYTIH | 89 |
| 10F4chim_VH wt_CDR2 | YINPSSGYTNYNQKFKA | 91 |
| 10F4chim_VH wt_CDR3 | MFRRSYFDY | 93 |
| 14H9chim_VH wt_CDR1 | TYGIH | 97 |
| 21B1chim_VH wt_CDR1 | TYGIH | 103 |
| 21B1chim_VH wt_CDR2 | VIWSDGSTIYNSALKS | 105 |
| 21B1chim_VH wt_CDR3 | HGGYYNYFDY | 107 |

TABLE 2
Sequences of the specific CDR domain of
the light variable domain of antibodies
according to the invention (according
to the Kabat system)

| | | SEQ ID |
|---|---|---|
| CDRs | Sequences | No: |
| 15E3chim_VL wt_CDR1 | SDSSSISSNYLH | 4 |
| 15E3chim_VL wt_CDR2 | GTSNLAS | 6 |
| 15E3chim_VL wt_CDR3 | QQGSSIPRT | 8 |
| 11H11chim_VL wt_CDR1 | RATENIYSYLA | 12 |
| 11H11chim_VL wt_CDR2 | NAKTLAE | 14 |
| 11H11chim_VL wt_CDR3 | QHHFGTPLT | 16 |
| 5D1chim_VL wt_CDR1 | HASQNINVWLS | 20 |
| 14H9chim_VL wt_CDR1 | HASQNINVWLS | 20 |
| 5D1chim_VL wt_CDR2 | KASNLHT | 22 |
| 14H9chim_VL wt_CDR2 | KASNLHT | 22 |
| 5D1chim_VL wt_CDR3 | QQGQSYWT | 24 |
| 14H9chim_VL wt_CDR3 | QQGQSYWT | 24 |
| 6C5chim_VL wt_CDR1 | RASESVDNHGFSFMN | 28 |
| 6C5chim_VL wt_CDR2 | AASNQGS | 30 |
| 6C5chim_VL wt_CDR3 | QQSKEVPWT | 32 |
| 10F4chim_VL wt_CDR1 | RSSQSLENSNGNTYLN | 36 |
| 10F4chim_VL wt_CDR2 | RVSNRFS | 38 |
| 10F4chim_VL wt_CDR3 | LQVTHVPYT | 40 |
| 21B1chim_VL wt_CDR1 | KSTQNLFYSTNQKNYLA | 49 |
| 21B1chim_VL wt_CDR2 | WASTRES | 51 |
| 21B1chim_VL wt_CDR3 | QQYYTYPWT | 53 |

Any combination of HCDR1, HCDR2, HCDR3, LCDR1, LCDR2 and LCDR3 is contemplated in the present invention.

TABLE 3
Sequences of heavy chain variable domains
of antibodies according to the invention

| Variable | | |
|---|---|---|
| heavy | | SEQ ID |
| chains | Sequences | No: |
| 15E3chim_VH wt | QVQLQQSGPELVKPGASVKMSCKASGFTFTDYVIAWVKVR | 55 |
| | TGQGLEWIGEIYPGSGSIYYNEKFKGKATLTADKSSNTAY | |
| | MQLSSLTSEDSAVYFCASSTVVAFDYWGQGTTLTVSS | |
| 11H11chim_VH wt | QIHLVQSGPELKKPGETVKISCKASGYTFTNFGMNWVKQA | 63 |
| | PGKGLKWMGWINTNTGEPTYADDFKGRFAFSLETSASTAY | |
| | LQINNLKNEDTATYFCARGAPAWFTYWGQGTLVTVSA | |
| 5D1chim_VH wt | QVQLKESGPGLVAPSQSLSITCTISGFSLTSYGVHWVRQP | 71 |
| | PGKGLEWLVVIWSDGSTIYNSALKSRLSISKDNSKSQVFL | |
| | KMNSLQTDDTAMYYCARHGGYYNYFDYWGQGTTLTVSS | |
| 6C5chim_VH wt | QVQLQQSGPELVKPGASVKMSCKASGYTFTDYVISWVKQK | 79 |
| | TGQGLEWIGEIYPGSGNTYYNEKFKGKATLTADKSSSTAY | |
| | IHLSSLTSEDSAVYFCAGGGSSHFDYWGQGTTLTVSS | |
| 10F4chim_VH wt | QVQLQQSGTELARPGASVKMSCKASGYIFTDYTIHWVKQR | 87 |
| | PGQGLEWVGYINPSSGYTNYNQKFKAKATLTADKSSSTAY | |
| | MQLSSLTSEDSAVYYCTPMFRRSYFDYWGQGTTLTVSS | |
| 14H9chim_VH wt | QVQLKESGPGLVAPSQSLSITCTISGFSLTTYGIHWVRQP | 95 |
| | PGKGLEWLVVIWSDGSTIYNSALKSRLSISKDNSKSQVFL | |
| | KMNSLQTDDTAIYYCARHGGYYNYFDYWGQGTTLTVSS | |
| 21B1chim_VH wt | QVQLKESGPGLVAPSQSLSITCTISGFSLTTYGIHWVRQP | 101 |
| | PGKGLEWLVVIWSDGSTIYNSALKSRLSISKDNSKSQVFL | |
| | KMNSLQTDDTAIYYCARHGGYYNYFDYWGQGTTLTVSS | |

TABLE 4
Sequences of light chain variable domains
of antibodies according to the invention

| Variable | | SEQ |
|---|---|---|
| light | | ID |
| chains | Sequences | No: |
| 15E3chim_VL wt | EIVLTQSPTTLAASPGEKIIITCSDSSSISSNYLHWYQQKPGFSPK | 2 |
| | LLIYGTSNLASGVPARFSGSGSGTTYSLTIGTMEAEDVATYYCQQG | |
| | SSIPRTFGGGTKLEIK | |
| 11H11chim_VL wt | DIQMTQSPASLSASVGETVTITCRATENIYSYLAWYQQKQGKSPQF | 10 |
| | LVYNAKTLAEGMPSRFSGSGSGTQFSLKINILQPEDFGTYYCQHHF | |
| | GTPLTFGAGTKLELK | |
| 5D1chim_VL wt | DIQMNQSPSSLSASLGDTITITCHASQNINVWLSWYQQKPGNIPKL | 18 |
| | LIYKASNLHTGVPSRFSGSGSGTGFTLTISSLQPEDIATYYCQQGQ | |
| | SYWTFGGGTKLEIK | |
| 6C5chim_VL wt | DIVLTQSPASLAVSLGQGATISCRASESVDNHGFSFMNWFQQKPGQ | 26 |
| | PPKLLIYAASNQGSGVPARFSGSGSGTDFSLNIHPMEEDDTAMYFC | |
| | QQSKEVPWTFGGGTKLEIK | |
| 10F4chim_VL wt | DAVMTQTPLSLPVSLGDQASISCRSSQSLENSNGNTYLNWYLQKPG | 34 |
| | QSPQLLIYRVSNRFSGVLDRFSGSGSGTDFTLKISRVEAEDLGVYF | |
| | CLQVTHVPYTFGGGTKLEIK | |
| 14H9chim_VL wt | DIQMNQSPSSLSASLGDTITITCHASQNINVWLSWYQQKPGNIPKL | 42 |
| | LTYKASNLHTGVPSRFSGSGSGTTFTLTIDSLQPEDIATYYCQQGQ | |
| | SYWTFGGGTKLEIK | |
| 21B1chim_VL wt | DIVMSQSPSSLAVSVGEKVTLTCKSTQNLFYSTNQKNYLAWYQQKP | 47 |
| | GQSPKLLIYWASTRESGVPDRFTGSGSGTDFTLTISSVKAEDPAVY | |
| | YCQQYYTYPWTFGGGTKLEIK | |

Any combination of one heavy chain variable domain selected among table 3 with one light chain variable domain selected among table 4 is contemplated in the present invention.

TABLE 5
Sequences of full chains of antibodies
of the invention

| | | SEQ |
|---|---|---|
| Full | | ID |
| chains | Sequences | No: |
| 15E3- | QVQLQQSGPELVKPGASVKMSCKASGFTFTDYVIAWV | 119 |
| VHwt- | KVRTGQGLEWIGEIYPGSGSIYYNEKFKGKATLTADK | |
| FcG4m | SSNTAYMQLSSLTSEDSAVYFCASSTVVAFDYWGQGT | |
| | TLTVSSASTKGPSVFPLAPCSRSTSESTAALGCLVKD | |
| | YFPEPVTVSWNSGALTSGVHTFPAVLQSSGLYSLSSV | |
| | VTVPSSSLGTKTYTCNVDHKPSNTKVDKRVESKYGPP | |
| | CPPCPAPEFLGGPSVFLFPPKPKDTLMISRTPEVTCV | |
| | VVDVSQEDPEVQFNWYVDGVEVHNAKTKPREEQFNST | |
| | YRVVSVLTVLHQDWLNGKEYKCKVSNKGLPSSIEKTI | |
| | SKAKGQPREPQVYTLPPSQEEMTKNQVSLTCLVKGFY | |
| | PSDIAVEWESNGQPENNYKTTPPVLDSDGSFFLYSRL | |
| | TVDKSRWQEGNVFSCSVMHEALHNHYTQKSLSLSPG | |
| | K* | |
| 11H11- | QIHLVQSGPELKKPGETVKISCKASGYTFTNFGMNWV | 120 |
| VHwt- | KQAPGKGLKWMGWINTNTGEPTYADDFKGRFAFSLET | |
| FcG4m | SASTAYLQINNLKNEDTATYFCARGAPAWFTYWGQGT | |
| | LVTVSAASTKGPSVFPLAPCSRSTSESTAALGCLVKD | |
| | YFPEPVTVSWNSGALTSGVHTFPAVLQSSGLYSLSSV | |
| | VTVPSSSLGTKTYTCNVDHKPSNTKVDKRVESKYGPP | |
| | CPPCPAPEFLGGPSVFLFPPKPKDTLMISRTPEVTCV | |
| | VVDVSQEDPEVQFNWYVDGVEVHNAKTKPREEQFNST | |
| | YRVVSVLTVLHQDWLNGKEYKCKVSNKGLPSSIEKTI | |
| | SKAKGQPREPQVYTLPPSQEEMTKNQVSLTCLVKGFY | |
| | PSDIAVEWESNGQPENNYKTTPPVLDSDGSFFLYSRL | |
| | TVDKSRWQEGNVFSCSVMHEALHNHYTQKSLSLSPG | |
| | K* | |
| 5D1- | QVQLKESGPGLVAPSQSLSITCTISGFSLTSYGVHWV | 121 |
| VHwt- | RQPPGKGLEWLVVIWSDGSTIYNSALKSRLSISKDNS | |
| FcG4m | KSQVFLKMNSLQTDDTAMYYCARHGGYYNYFDYWGQG | |
| | TTLTVSSASTKGPSVFPLAPCSRSTSESTAALGCLVK | |
| | DYFPEPVTVSWNSGALTSGVHTFPAVLQSSGLYSLSS | |
| | VVTVPSSSLGTKTYTCNVDHKPSNTKVDKRVESKYGP | |
| | PCPPCPAPEFLGGPSVFLFPPKPKDTLMISRTPEVTC | |
| | VVVDVSQEDPEVQFNWYVDGVEVHNAKTKPREEQFNS | |
| | TYRVVSVLTVLHQDWLNGKEYKCKVSNKGLPSSIEKT | |
| | ISKAKGQPREPQVYTLPPSQEEMTKNQVSLTCLVKGF | |
| | YPSDIAVEWESNGQPENNYKTTPPVLDSDGSFFLYSR | |
| | LTVDKSRWQEGNVFSCSVMHEALHNHYTQKSLSLSPG | |
| | K* | |
| 6C5- | QVQLQQSGPELVKPGASVKMSCKASGYTFTDYVISWV | 122 |
| VHwt- | KQKTGQGLEWIGEIYPGSGNTYYNEKFKGKATLTADK | |
| FcG4m | SSSTAYIHLSSLTSEDSAVYFCAGGGSSHFDYWGQGT | |
| | TLTVSSASTKGPSVFPLAPCSRSTSESTAALGCLVKD | |
| | YFPEPVTVSWNSGALTSGVHTFPAVLQSSGLYSLSSV | |
| | VTVPSSSLGTKTYTCNVDHKPSNTKVDKRVESKYGPP | |
| | CPPCPAPEFLGGPSVFLFPPKPKDTLMISRTPEVTCV | |
| | VVDVSQEDPEVQFNWYVDGVEVHNAKTKPREEQFNST | |
| | YRVVSVLTVLHQDWLNGKEYKCKVSNKGLPSSIEKTI | |
| | SKAKGQPREPQVYTLPPSQEEMTKNQVSLTCLVKGFY | |
| | PSDIAVEWESNGQPENNYKTTPPVLDSDGSFFLYSRL | |
| | TVDKSRWQEGNVFSCSVMHEALHNHYTQKSLSLSPG | |
| | K* | |
| 10F4- | QVQLQQSGTELARPGASVKMSCKASGYIFTDYTIHWV | 123 |
| VHwt- | KQRPGQGLEWVGYINPSSGYTNYNQKFKAKATLTADK | |
| FcG4m | SSSTAYMQLSSLTSEDSAVYYCTPMFRRSYFDYWGQG | |
| | TTLTVSSASTKGPSVFPLAPCSRSTSESTAALGCLVK | |
| | DYFPEPVTVSWNSGALTSGVHTFPAVLQSSGLYSLSS | |
| | VVTVPSSSLGTKTYTCNVDHKPSNTKVDKRVESKYGP | |
| | PCPPCPAPEFLGGPSVFLFPPKPKDTLMISRTPEVTC | |
| | VVVDVSQEDPEVQFNWYVDGVEVHNAKTKPREEQFNS | |
| | TYRVVSVLTVLHQDWLNGKEYKCKVSNKGLPSSIEKT | |
| | ISKAKGQPREPQVYTLPPSQEEMTKNQVSLTCLVKGF | |
| | YPSDIAVEWESNGQPENNYKTTPPVLDSDGSFFLYSR | |
| | LTVDKSRWQEGNVFSCSVMHEALHNHYTQKSLSLSPG | |
| | K* | |
| 14H9- | QVQLKESGPGLVAPSQSLSITCTISGFSLTTYGIHWV | 124 |
| VHwt- | RQPPGKGLEWLVVIWSDGSTIYNSALKSRLSISKDNS | |
| FcG4m | KSQVFLKMNSLQTDDTAIYYCARHGGYYNYFDYWGQG | |
| | TTLTVSSASTKGPSVFPLAPCSRSTSESTAALGCLVK | |
| | DYFPEPVTVSWNSGALTSGVHTFPAVLQSSGLYSLSS | |
| | VVTVPSSSLGTKTYTCNVDHKPSNTKVDKRVESKYGP | |
| | PCPPCPAPEFLGGPSVFLFPPKPKDTLMISRTPEVTC | |
| | VVVDVSQEDPEVQFNWYVDGVEVHNAKTKPREEQFNS | |
| | TYRVVSVLTVLHQDWLNGKEYKCKVSNKGLPSSIEKT | |
| | ISKAKGQPREPQVYTLPPSQEEMTKNQVSLTCLVKGF | |
| | YPSDIAVEWESNGQPENNYKTTPPVLDSDGSFFLYSR | |
| | LTVDKSRWQEGNVFSCSVMHEALHNHYTQKSLSLSPG | |
| | K* | |
| 21B1- | QVQLKESGPGLVAPSQSLSITCTISGFSLTTYGIHWV | 125 |
| VHwt- | RQPPGKGLEWLVVIWSDGSTIYNSALKSRLSISKDNS | |
| FcG4m | KSQVFLKMNSLQTDDTAIYYCARHGGYYNYFDYWGQG | |
| | TTLTVSSASTKGPSVFPLAPCSRSTSESTAALGCLVK | |
| | DYFPEPVTVSWNSGALTSGVHTFPAVLQSSGLYSLSS | |
| | VVTVPSSSLGTKTYTCNVDHKPSNTKVDKRVESKYGP | |
| | PCPPCPAPEFLGGPSVFLFPPKPKDTLMISRTPEVTC | |
| | VVVDVSQEDPEVQFNWYVDGVEVHNAKTKPREEQFNS | |
| | TYRVVSVLTVLHQDWLNGKEYKCKVSNKGLPSSIEKT | |
| | ISKAKGQPREPQVYTLPPSQEEMTKNQVSLTCLVKGF | |
| | YPSDIAVEWESNGQPENNYKTTPPVLDSDGSFFLYSR | |
| | LTVDKSRWQEGNVFSCSVMHEALHNHYTQKSLSLSPG | |
| | K* | |
| 15E3- | EIVLTQSPTTLAASPGEKIIITCSDSSSISSNYLHWY | 126 |
| VLwt- | QQKPGFSPKLLIYGTSNLASGVPARFSGSGSGTTYSL | |
| CLhk | TIGTMEAEDVATYYCQQGSSIPRTFGGGTKLEIKRTV | |
| | AAPSVFIFPPSDEQLKSGTASVVCLLNNFYPREAKVQ | |
| | WKVDNALQSGNSQESVTEQDSKDSTYSLSSTLTLSKA | |
| | DYEKHKVYACEVTHQGLSSPVTKSFNRGEC* | |
| 11H11- | DIQMTQSPASLSASVGETVTITCRATENIYSYLAWYQ | 127 |
| VLwt- | QKQGKSPQFLVYNAKTLAEGMPSRFSGSGSGTQFSLK | |
| CLhk | INILQPEDFGTYYCQHHFGTPLTFGAGTKLELKRTVA | |
| | APSVFIFPPSDEQLKSGTASVVCLLNNFYPREAKVQW | |
| | KVDNALQSGNSQESVTEQDSKDSTYSLSSTLTLSKAD | |
| | YEKHKVYACEVTHQGLSSPVTKSFNRGEC* | |
| 5D1- | DIQMNQSPSSLSASLGDTITITCHASQNINVWLSWYQ | 128 |
| VLwt- | QKPGNIPKLLIYKASNLHTGVPSRFSGSGSGTGFTLT | |
| CLhk | ISSLQPEDIATYYCQQGQSYWTFGGGTKLEIKRTVAA | |
| | PSVFIFPPSDEQLKSGTASVVCLLNNFYPREAKVQWK | |
| | VDNALQSGNSQESVTEQDSKDSTYSLSSTLTLSKADY | |
| | EKHKVYACEVTHQGLSSPVTKSFNRGEC* | |
| 6C5- | DIVLTQSPASLAVSLGQGATISCRASESVDNHGFSFM | 129 |
| VLwt- | NWFQQKPGQPPKLLIYAASNQGSGVPARFSGSGSGTD | |
| CLhk | FSLNIHPMEEDDTAMYFCQQSKEVPWTFGGGTKLEIK | |
| | RTVAAPSVFIFPPSDEQLKSGTASVVCLLNNFYPREA | |
| | KVQWKVDNALQSGNSQESVTEQDSKDSTYSLSSTLTL | |
| | SKADYEKHKVYACEVTHQGLSSPVTKSFNRGEC* | |
| 10F4- | DAVMTQTPLSLPVSLGDQASISCRSSQSLENSNGNTY | 130 |
| VLwt- | LNWYLQKPGQSPQLLIYRVSNRFSGVLDRFSGSGSGT | |
| CLhk | DFTLKISRVEAEDLGVYFCLQVTHVPYTFGGGTKLEI | |
| | KRTVAAPSVFIFPPSDEQLKSGTASVVCLLNNFYPRE | |
| | AKVQWKVDNALQSGNSQESVTEQDSKDSTYSLSSTLT | |
| | LSKADYEKHKVYACEVTHQGLSSPVTKSFNRGEC* | |
| 14H9- | DIQMNQSPSSLSASLGDTITITCHASQNINVWLSWYQ | 131 |
| VLwt- | QKPGNIPKLLIYKASNLHTGVPSRFSGSGSGTTFTLT | |
| CLhk | IDSLQPEDIATYYCQQGQSYWTFGGGTKLEIKRTVAA | |
| | PSVFIFPPSDEQLKSGTASVVCLLNNFYPREAKVQWK | |
| | VDNALQSGNSQESVTEQDSKDSTYSLSSTLTLSKADY | |
| | EKHKVYACEVTHQGLSSPVTKSFNRGEC* | |
| 21B1- | DIVMSQSPSSLAVSVGEKVTLTCKSTQNLFYSTNQKN | 132 |
| VLwt- | YLAWYQQKPGQSPKLLIYWASTRESGVPDRFTGSGSG | |
| CLhk | TDFTLTISSVKAEDPAVYYCQQYYTYPWTFGGGTKLE | |
| | IKRTVAAPSVFIFPPSDEQLKSGTASVVCLLNNFYPR | |
| | EAKVQWKVDNALQSGNSQESVTEQDSKDSTYSLSSTL | |
| | TLSKADYEKHKVYACEVTHQGLSSPVTKSFNRGEC* | |

Any combination of one heavy chain selected among table 5 with one light chain selected among table 5 is contemplated in the present invention. In a particular embodiment, the combination of a heavy chain and a light chain sharing the same reference name is contemplated.
The following Figures and Examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

### BRIEF DESCRIPTION OF THE FIGURES

FIG. 1 illustrates a phagocytosis assay of tumor cells (non-Hodgkin's lymphoma cells (Raji cells) (A) and non small cell lung cancer (NSCLC) (B)) in presence of chimeric antibodies according to the present invention (5D1, 6C5, 11H11, 14H8 and 15E3) as compared with an anti-CLEC-1A antibody of the prior art (αCLEC-1 Ctrl mAb) which corresponds to the anti-CLEC-1A antibody disclosed in WO2018073440A1 and Robles et al. (Blood advances, 2017), and an isotype control antibody (h1G4). Ratio of phagocytosis was determined by normalizing the frequency of Clec-1 blocked TGFb-DC that have phagocytosed tumor cells over the PBS or hIgG4 controls according the isotype of the used mAb. A: Assay on Raji cells; B: Assay on Non Small Cell Lung Cancer cells.

FIG. 2 illustrates a phagocytosis assay of UV-treated hepatocarcinoma model tumor cells by TGFb-DC blocked in presence of an anti-CLEC-1A antibody of the prior art (αCLEC-1 Ctrl mAb) which corresponds to the anti-CLEC-1A antibody disclosed in WO2018073440A1 and Robles et al. (Blood advances, 2017), and anti-CLEC-1A antibodies of the invention (murine antibody 5D1A5 and chimeric antibody 14H9 and 6C5).A hIgG4 isotype control antibody was used. Ratio of phagocytosis was determined by normalizing the frequency of Clec-1 blocked TGFb-DC that have phagocytosed tumor cells over the PBS or hIgG4 controls according the isotype of the used mAb.

FIG. 3 illustrates a phagocytosis assay of tumor cells by macrophages in presence of chimeric anti-CLEC-1A antibodies of the invention (6C5, 14H9), and compared with the results obtained with an anti-CLEC-1A antibody of the prior art (αCLEC-1 Ctrl mAb) which corresponds to the anti-CLEC-1A antibody disclosed in WO2018073440A1 and Robles et al. (Blood advances, 2017), associated with different targeting-tumor antibodies (rituximab, cetuximab or trastuzumab). A hIgG4 isotype control antibody was used. Ratio of phagocytosis was determined by normalizing the frequency of phagocytosed cells as compared to the results observed with the isotype antibody control. A; Assay on Raji cells, a model of non-Hodgkin's lymphoma. B. Model of a colorectal cancer. C. Model of Breast cancer.

FIG. 4 is a table representing the IC50 determination from FIGS. 7 and 9. IC50 refers to the concentration required to inhibit 50% of the signal in this assay for each murine anti-hCLEC1 antibodies.

FIG. 5 illustrates an antagonist activity study of anti-CLEC1 antibodies on Fc-CLEC1-permeabilised Raji interaction by FACS: The different anti-CLEC1 antibodies were tested over a dose response: 15E3, 5D1, 6C5, 14H9, 11H11 (chimeric anti-CLEC-1A antibodies of the invention) and isotype IgG1 and in-house positive control chimeric anti-CLEC1 (non-antagonist) in negative controls .The curve represent the percentage of binding of Fc-CLEC1-A488 at 10 nM on Raji cells after competition with anti-CLEC1 antibodies.

FIG. 6 illustrates the IC50 determination issued from the experiment illustrated on FIG. 5. The IC value refers to the concentration required to inhibit 50% of the signal in this assay for each chimeric anti-hCLEC1 antibodies. Positive control is an in-house chimeric anti-CLEC1 antibody.

FIG. 7 illustrates an antagonist activity study of anti-CLEC1 antibodies (including chimeric anti-CLEC-1A antibodies of the invention 15E3, 5D1, 6C5, 14H9) on Fc-CLEC1-permeabilized PBMC interaction by FACS. The curve illustrates the percentage of binding of Fc-CLEC1-A488 at 10 nM on PBMC after competition with anti-hCLEC1 antibodies.

FIG. 8 represents the IC50 determination from the experiment of FIG. 7. The IC value refers to the concentration required to inhibit 50% of the signal in this assay for each chimeric anti-hCLEC1 antibodies.

FIG. 9 illustrates the productivity of selected anti-CLEC-1A antibodies, including chimeric anti-CLEC-1A antibodies of the invention 15E3, 5D1, 6C5, 14H9, 11H11, in HEK cells. The antibodies correspond to the combination specific heavy and light variable domains disclosed in the examples of the invention.

FIG. 10 illustrates the productivity of selected anti-CLEC-1A antibodies, including chimeric anti-CLEC-1A antibodies of the invention 15E3, 5D1, 6C5, 14H9, 11H11, in HEK and CHO cells. The antibodies correspond to the combination specific heavy and light variable domains disclosed in the examples of the invention. “nd” corresponds to an absence of productivity data for the specified antibody in the specified cell line.

FIG. 11 illustrates the ED50 determined from a binding assay between each murine anti-hCLEC1 antibodies and human CLEC-A-his. The clone ID corresponds to specific combinations of a heavy chain variable domain and a light chain variable domain, as disclosed in the description of the invention. ED50 refers to the concentration required to reach 50% of the maximal OD signal in this assay for each anti-CLEC1A antibody.

FIG. 12 illustrates a binding study of chimeric CLEC1A antibodies (15E3, 5D1, 6C5, 14H9) by ELISA. The curves represent the binding to His-CLEC1 of chimeric anti-CLEC1A antibodies at different concentrations (ng/ml): positive control chimeric anti-CLEC1A (non-antagonist control), 15E3, 5D1, 6C5, 14H9 chimeric antibodies

FIG. 13 represents ED50 determination from FIG. 12; ED50 refers to the concentration required to reach 50% of the maximal signal in this assay for each chimeric anti-hCLEC1A antibodies.

FIG. 14 illustrates a binding study of chimeric CLEC1 antibody 11H1lby ELISA. The curves represent the binding to His-CLEC1 of chimeric anti-CLEC1 antibody 11H11 at different concentrations (ng/ml); positive control is an in-house chimeric anti-CLEC1 antibody.

FIG. 15 represents ED50 determination from FIG. 14; ED50 refers to the concentration required to reach 50% of the maximal signal in this assay for each chimeric anti-hCLEC1 antibodies.

FIG. 16 illustrates the binding affinity (KD), the affinity constant (ka) and the dissociation constant (kd) of anti-CLEC1 murine antibodies of the invention for human CLEC-A-his recombinant protein measured by Blitz.

FIG. 17 illustrates the binding affinity (KD), the affinity constant (ka) and the dissociation constant (kd) of anti-CLEC1 chimeric antibodies of the invention for human CLEC-A-his recombinant protein measured by Blitz.

FIG. 18 illustrates the ED50 determination of a binding study of murine CLEC1 antibodies of the invention on human U266 cell lines by Flow cytometry (FACS) by ELISA ED50 refers to the concentration required to reach 50% of the signal in this assay for each anti-CLEC1 antibody.

FIG. 19 illustrates a binding study of chimeric CLEC1 antibodies of the invention on human U266 cell lines by Flow cytometry (FACS) by ELISA: A: represents the percentage of stained U266; B: represents the Mean Fluorescence Intensity (MFI) of the different antibodies over a dose response.

FIG. 20 illustrates a binding study of chimeric CLEC1 antibodies of the invention on human U266 cell lines by Flow cytometry (FACS) by ELISA (A). The result illustrates the EC50 binding of the chimeric antibodies according to the invention on U266 cells. B is a table illustrating the EC50 determination from the curves; EC50 refers to the concentration required to reach 50% of the signal in this assay for each chimeric anti-hCLEC1 antibodies.

FIG. 21 illustrates the binding of chimeric anti-CLEC-1A antibodies of the invention on CHO cells analysed by FACS cytometry: A: represents the percentage of stained CHO cells over a dose response; B: represents the Mean Fluorescence Intensity (MFI) of the different antibodies over a dose response.

FIG. 22 is a table illustrating the EC50 value issued from FIG. 21. EC50 refers to the concentration required to reach 50% of the signal in this assay for each anti-CLEC1 antibody.

FIG. 23 illustrates a competitive activity study of anti-CLEC1 antibodies on His-CLEC1 interaction by ELISA. The different anti-CLEC1 antibodies were tested at 1 μg/mL for the competitor and 10 ng/mL to 2 μg/mL for the challenged antibody: 15E3, 5D1, 6C5, 14H9, 11H11 (chimeric anti-CLEC1A antibodies of the invention) and isotype IgG1 and positive control chimeric anti-CLEC1 (non-antagonist) in negative controls. The histograms represent the binding to His-CLEC1 after competition with anti-CLEC1 antibodies.

FIG. 24 illustrates a competitive activity study of anti-CLEC1 antibodies on His-CLEC-1A interaction by ELISA with a selected antibody according to the invention (11H11). 11H1lanti-CLEC1 antibody was tested at 111g/mL and challenged antibodies were added at and 10 ng/mL to 2 μg/mL: 11H11 (A), 5D1 (B), 15E3 (C), 14H9 (D), 6C5 (E) (chimeric anti-CLEC1A antibodies of the invention).

### MATERIAL AND METHODS

### Preparation, Selection and Characterisation of Novel Anti Human CLEC Monoclonal Antibodies

Mice were immunized with His Clec-1 (recombinant human CLEC-1 protein with His Tag, #1704-CL R&D Systems, Minneapolis, USA) or Fc-Clec (recombinant human CLEC-1 protein fused with a constant fragment of human immunoglobulin IgG1 at N-Terminal domain—Osé Immunotherapeutics, Nantes, France) and monoclonal antibodies were derived according to conventional techniques. The immunization protocol was performed by Diaclone SAS (Besançon, France): recombinant His-Clec protein—or recombinant Fc-Clec—was used to immunize 3 BALB/c strain mice. One microgram of proteins was administered in foot pad, one day per week for the first three injections and one day per two weeks for the two last injections. The fifth injection at 42 days was considered as a boost before collecting ganglion cells. Hybridoma were obtained by fusing ganglion cells with the mouse myeloma X63/AG.8653. Hybridoma were first screened according to the capacity of the secreted monoclonal antibodies to bind specifically the recombinant His-Clec-1 protein (#1704-CL, R&D System) and Clec-Fc (recombinant human CLEC-1 protein fused with human IgG1 at C-terminal domain), and to bind specifically Clec protein at the surface of human myeloma U266 cells. After selection, hybridoma were cloned and cultured in DMEM complete medium. Supernatant was purified by affinity on Protein A chromatography (DiaClone, Besançon, France) with glycine 0.1 M pH 2.8 elution buffer). Activity of purified antibodies purified were measured in ELISA against Clec-1 human proteins and flow cytometry assay against U266 cells.

### Human His-Clec, Human Clec-Fc and Mouse Fc-Clec Binding by ELISA

For activity ELISA assay, recombinant His-Clec (#1704-CL R&D Systems, Minneapolis, USA)—or recombinant human Clec-Fc (Ose Immunotherapeutics, Nantes)—was immobilized on plastic at 2 μg/ml in carbonate buffer (pH 9.2) and purified antibodies were added to measure binding. After incubation and washing, peroxidase-labeled donkey anti-mouse IgG (#715-036-151, Jackson Immunoresearch, USA) was added and revealed by conventional methods. For cross-specificity on mouse, recombinant mouse Clec-Fc (recombinant mouse Clec fused with a constant fragment of mouse IgG1 at N-terminal, Osé Immunotherapeutics, Nantes) was immobilized on plastic at 2 μg/ml in carbonate buffer (pH 9.2) and purified antibodies were added to measure binding. After incubation and washing, a goat anti-mouse IgG kappa chain (#115-005-174, Jackson Immunoresearch, USA) followed by a peroxidase-labeled donkey anti-goat IgG (#705-035-147, Jackson Immunoresearch, USA) was added and revealed by conventional methods.

### Biosensor Affinity Measurement for Clec-1 by BLITZ

Analysis was performed with a BLItz System (Fortébio, #C22-2 No 61010-1, Molecular Devices, San José, USA). Recombinant His-Clec (#1704-CL R&D Systems, Minneapolis, USA) was immobilized on a Ni-NTA bio-sensor (Fortébio, #18-0029) at 10 μg/ml during 2min. Values were measured after an association period (ka) of 3 min 120 sec of 20 μg/ml of purified antibodies followed by a dissociation period of 10 min 120 sec (kd) to determine affinity constant (KD).

### Clec-1 Binding Assay on Human U266 Myeloma by Cytofluorometry

To measure binding of anti-Clec-1 on human U266 myeloma, cells were first washed in cold-PSE (Phosphate Buffer Saline with 2% of heat-inactivated bovine serum, 2 mM EDTA) and incubated for 30 min on ice to slow cell metabolism. Then, antibodies in concentration range were incubated for 30 min on ice, and cells were washed with cold-PSE before staining for 30 min on ice with an Alexa 647-labelled goat anti-mouse IgG at 5 μg/ml (#A21236; Fisher Scientific, Illkirch, France). Samples were analyzed on CytoFlex cytofluorometer (Beckman Coulter France, Villepinte).

### Antagonist Activity by FACS (Flow Cytometry)

For competitive assay, Fc-Clec-1 (Ose Immunotherapeutics, Nantes, France) were coupled with Alexa Fluor 488 (Alexa Fluor® 488 Microscale Protein Labeling Kit #A30006, Fisher Scientific, Illkirch, France).

Permeabilized (#554714 CytoFix/Cytoperm kit, BD Biosciences, Le Pont de Claix, France) and Fc-blocked (#564220, BD Biosciences) Burkitt lymphoma Raji cells express a Clec-1 ligand, which can be detected after incubation with Alexa488-Fc-Clec-1 at 10 nM or 20 nM. For permeabilization (#554714 CytoFix/Cytoperm kit, BD Biosciences, Le Pont de Claix, France), cells were first washed in cold-PSE, incubated in cold Cytofix reagent during 20 min on ice and washed in Perm Wash buffer (1/10 in water). For Fc-blocking, cells were then incubated during 30 min at room temperature with human Fc-Block (#564220, BD Biosciences) diluted 50-fold in Perm Wash Buffer.

To measure competition, purified anti-Clec antibodies at different concentrations were pre-incubated in Perm Wash Buffer with Alexa488-Fc-Clec-1 for 15 min at room temperature. Pre-incubated mixes were then incubated on permeabilized and Fc-blocked Raji cells for 30 min on ice. Binding on cells were then fixed by PFA 2% in cold PBS for 10 min on ice and analyzed on CytoFlex cytofluorometer (Beckman Coulter France, Villepinte).

Competition were also measured on UV pre-treated pro-apoptotic human PBMCs, which can also express the ligand after a treatment under UV radiation for 18 hours. After UV treatment, cells were first washed in cold-PSE and then blocked during 30 min at room temperature with human Fc-block 50-fold diluted in PSE, and then replaced on ice. Purified anti-Clec antibodies at different concentrations were pre-incubated with Alexa488-Fc-Clec-1 in PSE at different concentrations for 15 min at room temperature. Pre-incubated mixes were then incubated on Fc-blocked/SAB (#34005-100, Invitrogen, Illkirch, France) pro-apoptotic PBMCs for 30 min on ice. Binding on cells were then fixed by PFA 2% in cold PBS for 10 min on ice and analyzed on CytoFlex cytofluorometer (Beckman Coulter France, Villepinte).

### Race PCR of Nucleotides and Amino Acid Sequences of Anti-Human Anti-Clec Mabs

VH and VL regions of the anti-clec clones were sequenced using the 5′ RACE PCR technology (Sigma reference 3353621001). Briefly, total RNA was extracted by Trizol method, reverse transcribed and the resulting cDNA was poly-adenylated at the 5′ end of the molecules using dATP and the terminal transferase enzyme. A first 35-cycle PCR reaction was performed using a oligo dT anchor primer and a constant region binding primer with a Herculase enzyme (Agilent reference 600679). A second 35-cycle PCR was performed using nested PCR anchor primers and nested primer of constant region. The resulting PCR product was then TA-cloned in E. Coli and after selection on ampicillin and on X-gal, resulting white colonies were screened by PCR using nested PCR anchor primer and nested primer of constant region and inserted cDNA sequenced. Nucleotidic sequences and deduced amino acid sequences are shown on Figure x and in the Sequence Listing.

### Preparation and Characterisation of Chimeric Anti-Clec Antibodies

For chimeric anti-clec, variable sequence of heavy chain (VH) of mouse anti-clec antibodies was cloned by EcoRV in pcDNA3.4 human G4m expression plasmid (OSE immunotherapeutics plasmid) containing CH1-hinge-CH2-CH3 domains of hIgG4, mutated at S228P to stabilize hinge region. Variable sequence of light chain (VL) of mouse anti-clec antibody was cloned by BsiWI in pcDNA3.4 CLIg-hkappa expression plasmid (OSE immunotherapeutics plasmid) containing human CLkappa.

First in COS cells, we have co-transfected, by lipofectamine method, plasmid containing VH-hFcG4m with plasmid containing VL-CLk. After 48-72 h incubation, supernatant was recovered. For a first screening, they were quantified by sandwich ELISA and tested in activity assay against Clec-his ELISA.

Then in HEK freestyle cells, we have co-transfected same plasmid containing VH-hFcG4m and VL-CLhk by lipofectamine method. After 6 days incubation, supernatant was recovered and purified by affinity on Protein A chromatography (HiTrap, GeHealthcare) with citric acid 0. 1 M pH 3 elution buffer. Purified antibody was dialyzed in PBS and concentrated. They were quantified by UV nanodrop and tested in several test: activity assay against Clec-his in ELISA and in Blitz (ForteBio), activity assay against U266 cells (Clec is present on the surface of cells), antagonist assay with permeabilized PBMC and Raji cells.

### Preparation, Characterization and Production of Chimeric Anti-CLEC1 Antibodies

Method. Mice were immunized using recombinant protein human CLEC1-His (R&D Systems reference 1704-CL) or Fc-hCLEC1 (OSE Immunotherapeutics) to generate murine monoclonal antibodies directed against this antigen. Intramuscular injections were administered in foodpad in five anesthetized BALB/c adult mice. All mouse experiments were performed in accordance with national guidelines. Mice were immunized according to a protocol of immunization with a minimum of 5 injections of 1 μg were performed at different time until 42 days after the primo injection. These mice received a boost pre melting before fusion with myeloma to generate hybridoma. Screening of interest hybridoma was made with binding assays and antagonist assays, as mentioned below, before used for the production of mAbs. This immunization campaign has resulted in the selection of six antagonist murine antibodies. VH and VL regions of the six clones (14H9-F3, 5D1-A5, 11H11-G11, 10F4-H2, 6C5-A4, 15E3-G3) were sequenced using the 5′ RACE PCR technology. Amino acid sequences and CDR description are disclosed in the Sequence Listing

For construction of heavy chain of anti-CLEC1 Ab, variable domain VH sequence were synthetized and cloned by EcoRV in pcDNA3.4-hIgG4m expression plasmid (Ose Immunotherapeutics) containing human Fc of human IgG4 mutated S228P to prevent fab-arm exchange. For construction of light chain of anti-CLEC1 Ab, variable domain VL were synthetized and cloned by BsiWI in pcDNA3.4-CLlghk expression plasmid containing human CLkappa (Ose Immunotherapeutics). In mammalian HEK cells, we have co-transfected, by lipofectamine method, plasmids containing VH-hFcG4m with plasmid containing VL-CLkappa. After 5-6 days incubation, supernatant was recovered and quantified by sandwich ELISA assay. Supernatant could be purified by affinity on Protein A chromatography (HiTrap, GeHealthcare) with citric acid 0.1 M pH 3 elution buffer. Purified antibody was dialyzed in PBS and concentrated. They were quantified by UV (A280 nm) and tested in activity assay against His-CLEC1 in ELISA assay.

### Sandwich ELISA for Quantitation AnTibody in Supernatant

For quantitation ELISA assay, donkey anti-human IgG, Fc specific (Jackson Immunoresearch; USA; reference 709-005-098) was immobilized on plastic at 1.3 μm/ml in borate buffer (pH9) and supernatants containing antibody were added to measure binding, compared to standard antibody. After incubation and washing, mouse anti-human kappa antibody (Ose Immunotherapeutics, reference NaM76-5F3) was added and detected by peroxidase-labeled donkey anti-mouse IgG antibody (Jackson Immunoresearch; USA; reference 715-036-151). Revelation of ELISA was made by conventional methods

### ELISA Activity Assay Human Anti-CLEC-1A

For activity ELISA assay, recombinant hCLEC-His (R et D systems; reference 1704-CL) was immobilized on plastic at 2 μg/ml and supernatants containing antibodies or purified antibodies were added to measure binding. After incubation and washing, peroxidase-labeled donkey anti-human IgG (Jackson immunoresearch reference 709-035-149) was added and revealed by conventional methods.

### Clec-his Binding Assay on U266 Cells by Cytofluorometry

To measure binding of anti-clec on U266 cells, human Fc Receptor Binding Inhibitor diluted at 1/200 (BD pharmingen; USA; reference 564220) was first added for 30 min at room-temperature to block human Fc receptors on U266 cells to reduce background. Then, antibodies were incubated for 30 min at 4° C., and washed before stained 15 min at 4° C. with PE-labelled anti-human IgG Fc (Biolegend; USA; reference 409303). Samples were analyzed on citoflex (Beckman coulter).

### Clec Binding Assay on CHO-Clec-1 Cells (Cells Transduced with a Clec-1 Expressing Lentivirus) by Cytofluorometry

To measure binding of anti-clec on CHO-Clec-1 cells, human Fc Receptor Binding Inhibitor diluted at 1/200 (BD pharmingen; USA; reference 564220) was first added for 30 min at room-temperature to block human Fc receptors on CHO-Clec-1 cells to reduce background. Then, antibodies were incubated for 30 min at 4° C., and washed before stained 15 min at 4° C. with PE-labelled anti-human IgG Fc (Biolegend; USA; reference 409303). Samples were analyzed on citoflex (Beckman coulter).

### Affinity Analysis by Blitz of Anti-Clec Antibodies on Human CLEC-His Recombinant Protein

Clec-His recombinant protein (R et D systems; reference 1704-CL) was immobilized onto a NINTA biosensor and the indicated antibodies were added at 20 μg/ml. Values were deduced after an association period (ka) of 12 0sec followed by a dissociation period of 120 sec (kd) to determine affinity constant (KD).

### Phagocytosis Assay

Monocytes were isolated by magnetic sorting from cytapheresis of healthy volunteers using Classical Monocytes Isolation kit provided by Miltenyi. Then, monocytes were cultured for 6-7 days with 50 ng/mL of human recombinant GM-CSF (CellGenix) and 20 ng/mL of human recombinant IL-4 (CellGenix) in order to generate immature dendritic cells (iDC). iDCs were polarized into immunotolerant DCs with 50ng/mL of human recombinant TGFb (PeproTech) for 2 days, which leads to overexpression of Clec-1 by these TGFb-DCs. Antibodies were added during the polarization at 10 μg/mL. TGFI3-DC were cultured with the non-Hodgkin's lymphoma (Raji) at a 1:1 ratio with the anti-CD20 mAb (Rituximab) at 10 ng/mL providing the “Eat-me” signal; the bare NSCLC cells (A549) were cultured for 5 hours with TGFIβ-DC. Phagocytosis was analyzed by flow cytometry and normalized over the control antibody condition for each donor.

Macrophages (MΦ) were generated from monocytes with M-CSF (10Ong/mL) for 5 days. MΦ were cultured with either the non-Hodgkin's lymphoma (Raji; CD20+) or the colon carcinoma (DLD-1; EGFR2+), or the breast cancer (SK-BR3; Her2+) at a 1:2 ratio +/− either the anti-CD20 mAb (Rituximab), the anti-EGFR mAb (Cetuximab), or the anti-Her2 mAb (Trastuzumab) respectively at 10 ng/mL providing the “Eat-me” signal, for 2 hours. Phagocytosis analysis was performed by flow cytometry and the percentage of phagocytosis was calculated by the percentage of CPDe670+ cells in total CPDe450+ cells. Results were expressed by multiplying the percentage of M1 that have phagocytosed Raji cells with the median intensity fluorescence of phagocytic cells and represented according the Rituximab concentration.

For the macroscopy assay, the macrophages were generated as described above. MO were preincubated with the anti-CLEC1 chimeric mAbs for 2 hours and then cultured with the non-Hodgkin's lymphoma (Raji; CD20+)+the anti-CD20 mAb (Rituximab) respectively at 10 ng/mL providing the “Eat-me” signal, for 4 hours. Phagocytosis analysis was performed by microscopy and the percentage of phagocytosis was calculated by the percentage of pHrodo (pHrodo-SE, Thermofisher) positive Raji in total Macrophages.

Tumor cell lines, Raji (B lymphoma) CSCLC cells, colorectal cancer cells and breast cancer cells Huh7 (Hepatocarcinoma), were stained with a fluorescent dye to characterize the cells in the phagocytosis assay. Briefly, tumor cells were incubated with the Cell Proliferation Dye eFluor 670 for 15 min and washed before UV treatment according the manufacturer's instructions (Life Technologies). Then, cells were treated with UV at 150 mJ/cm² and incubated overnight to trigger the apoptotic induced program which leads to Clec-1 ligand expression. TGFb-DC and tumor cell lines were collected, numbered and incubated at two DC for one tumor cells ratio for 5 hours and antibody were added during this process at 10 μg/mL. Phagocytosis was evaluated by flow cytometry on CPD-eFluor670 positive TGFb-DC.

In the examples of the invention, except when specifically noted, the anti-CLEC-1A control antibody is an in-house antibody that has no antagonist properties.

### EXAMPLES OF THE INVENTION

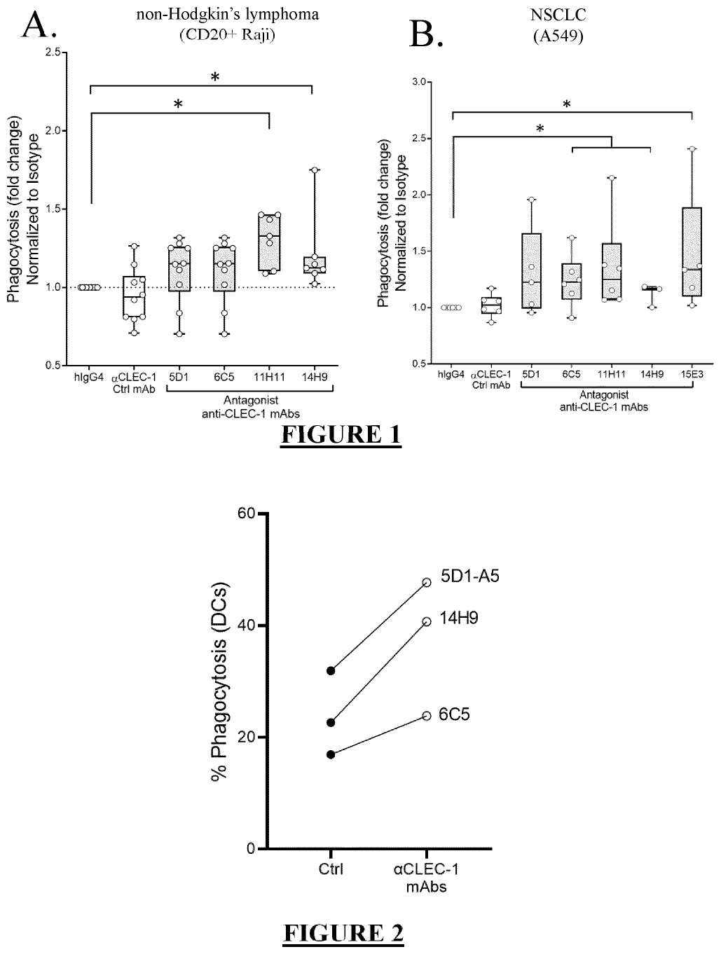

### Example 1. Biological Activity of Mouse and Chimeric Anti-hCLEC1A Antagonistic Antibodies of the Invention and of the Anti-hCLEC1A Antagonistic Antibody Disclosed in the Prior Art on Dendritic Cell Tumoral Phagocytosis—FIGS. 1-2

### Methods.

a) Generation of Monocytes Derived Dendritic cells (DC) Polarized with TGFb Recombinant Protein

Monocytes were isolated by magnetic sorting from cytapheresis of healthy volunteers using Classical Monocytes Isolation kit provided by Miltenyi. Then, monocytes were cultured for 6-7 days with 50 ng/mL of human recombinant GM-CSF (CellGenix) and 20 ng/mL of human recombinant IL-4 (CellGenix) in order to generate immature dendritic cells (iDC). iDCs were polarized into immunotolerant DCs with 50 ng/mL of human recombinant TGFb (PeproTech) for 2 days, which leads to overexpression of Clec-1 by these TGFb-DCs. Antibodies were added during the polarization at 10 μg/mL.

### b) Generation of UV-Treated Apoptotic Tumor Cell Lines

Tumor cell lines, Raji (B lymphoma) NSCLC cells, colorectal cancer cells, breast cancer cells or Huh7 (Hepatocarcinoma), were stained with a fluorescent dye to characterize the cells in the phagocytosis assay. Briefly, tumor cells were incubated with the Cell Proliferation Dye eFluor 670 for 15 min and washed before UV treatment according the manufacturer's instructions (Life Technologies). Then, cells were treated with UV at 150 mJ/cm² and incubated overnight to trigger the apoptotic induced program which leads to Clec-1 ligand expression.

### c) Phagocytosis Assay

TGFb-DC and tumor cell lines were collected, numbered and incubated at two DC for one tumor cells ratio for 5 hours and antibody were added during this process at 10 μg/mL. Phagocytosis was evaluated by flow cytometry on CPD-eFluor670 positive TGFb-DC. Results. FIG. 1 illustrates the phagocytosis of UV treated tumor cells by TGFb-DC normalized over the control conditions. In two different models of cancers, lymphoma (FIG. 1A) and Non Small cell Lung Cancer (FIG. 1B) the antagonistic chimeric 5D1, 14H9,6C5, 11H11 and 15E3 antibodies of the invention increased the phagocytosis of tumor cells whereas control antibody of the prior art (disclosed in W02018073440A1 and Robles et al. (Blood advances, 2017)) did not induce any significant change on the DC ability to phagocyte tumor cells. FIG. 2 illustrates the phagocytosis of UV treated tumor cells by TGFb-DC normalized over the control conditions in a hepatocarcinoma model of cancer. Three antibodies according to the present invention (murine antibody 5D1-A5, chimeric antibodies 14H9, and 6C5) increase the phagocytosis of tumor cells by DC, contrary to the antibody of the prior art. This example demonstrates the capability of the antibodies of the invention to increase the phagocytosis of tumor cells by dendritic cells, contrary to the antibody of the prior art (disclosed in WO2018073440A1 and Robles et al. (Blood advances, 2017)).

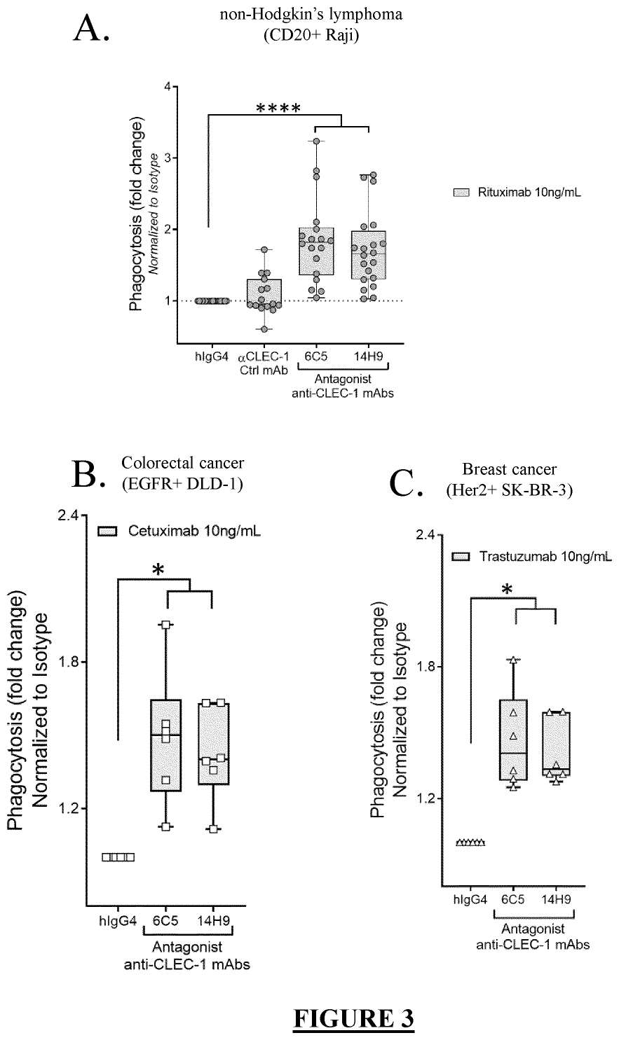

### Example 2. Biological Activity of a Combination of Anti-hCLEC-1A Antagonistic Antibody of the Invention or of the Anti-hCLEC1A antagonistic Antibody of the Prior Art with a Tumor-Targeting Antibody: Rituximab, Cetuximab or Trastuzumab—FIG. 3

### Methods.

Macrophages (MΦ) were generated from monocytes with M-CSF (100 ng/mL) for 5 days. MΦ were cultured with either the non-Hodgkin's lymphoma (Raji; CD20+) or the colon carcinoma (DLD-1; EGFR2+), or the breast cancer (SK-BR3; Her2+) at a 1:1 ratio +/− either the anti-CD20 mAb (Rituximab), the anti-EGFR mAb (Cetuximab), or the anti-Her2 mAb (Trastuzumab) respectively at 10 ng/mL providing the “Eat-me” signal, for 2 hours. Phagocytosis was analyzed by flow cytometry, and microcopy for Raji cells, and normalized over the control antibody condition for each donor or depicted as percentage of phagocytosed cells according the analysis. * p<0.05

Phagocytosis analysis was performed by flow cytometry and the percentage of phagocytosis was calculated by the percentage of CPDe670+ cells in total CPDe450+ cells. Results were expressed by multiplying the percentage of M1 that have phagocytosed Raji cells with the median intensity fluorescence of phagocytic cells and represented according the Rituximab concentration.

Results: The phagocytosis assay shows that M1 macrophages are able to phagocyte Raji cells, in presence of a combination of Rituximab and an anti-CLEC-1A antibody of the invention, as compared to the antibody of the prior art (disclosed in WO2018073440A1 and Robles et al. (Blood advances, 2017)). The same results are illustrated for two other cancer model; phagocytosis of colorectal cancer tumor cells by macrophages is increased when a combination of Cetuximab and an anti-CLEC-1A antibody of the invention is administered; and phagocytosis of breast cancer tumor cells by macrophages is increased when a combination of Trastuzumab and an anti-CLEC-1A antibody of the invention is administered. The combination of the anti-CLEC-1A antibodies of the invention with a second anti-tumor antibody enhances the phagocytosis capability of macrophages M1. It is therefore illustrated that using an anti-CLEC-1A antagonist antibody of the invention enhances the therapeutic effects of tumor targeting antibodies.

This example demonstrates the capability of the antibodies of the invention in combination with a tumor-targeting antibody to increase the phagocytosis of tumor cells by macrophages, contrary to the antibody of the prior art (disclosed in WO2018073440A1 and Robles et al. (Blood advances, 2017)).

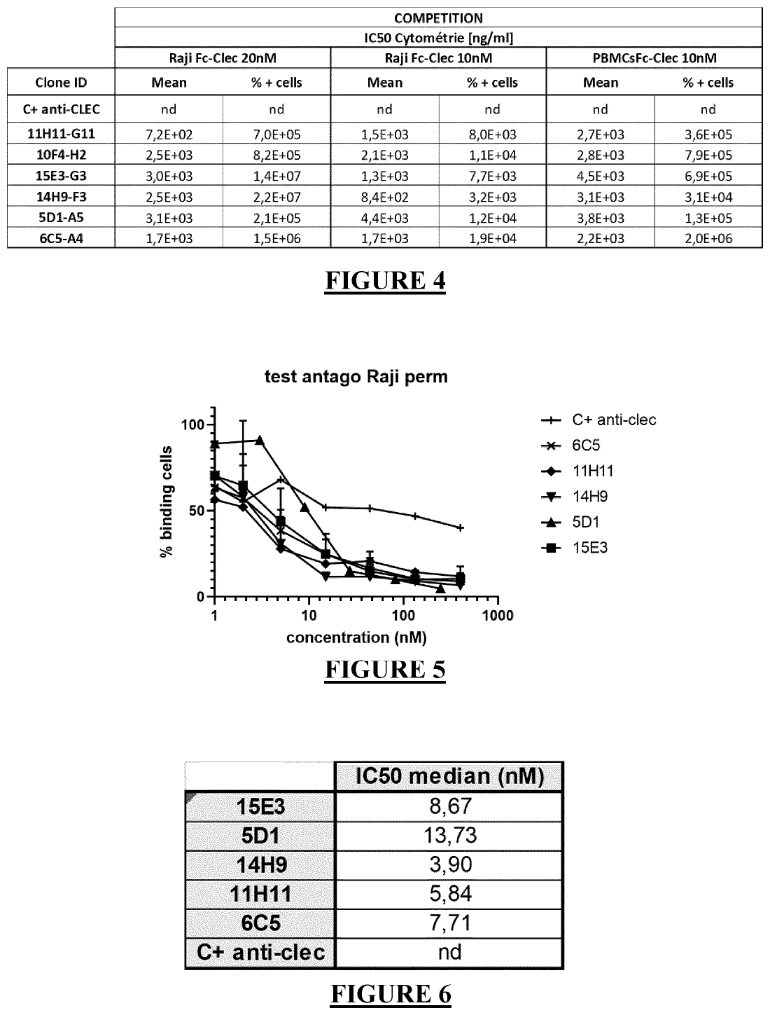

### Example 3: Competitive Study Between CLEC1-Ligand and Murine or Chimeric Anti-hCLEC1 Antibodies Using Antagonist Assays—FIGS. 4 to 8

Methods. To measure competition on permeabilised Raji (CytoFix/cytoperm kit, BD Biosciences) which expressed CLEC1 ligand, Fc-CLEC1-labelled A488 which bound specifically to permeabilised Raji was used. To measure competition, Fc-CLEC1 labelled A488 at 10 nM or 20 nM was mixed with mouse anti-hCLEC1 at different concentrations for 15 min at RT then added on these cells for 30 min at 4° C. After incubation and washing, PFA 2% was added to wells to fix cells for 10 min at 4° C. and analyzed on CytoFlex (Beckman) cytofluorometer to detect the inhibition of Fc-CLEC1-labelled. To measure competition on PMBC, we used human PBMC pre-treated by UV for 18 h to obtain apoptotic PBMC, which expressed CLEC1 ligand. Fc-CLEC1-labelled A488 bound specifically apoptotic UV-treated PBMC. To measure competition, Fc-CLEC1 labelled A488 at 10 nM was mixed with mouse anti-hCLEC1 at different concentrations for 15 min at RT then added on these cells for 30 min at 4° C. After incubation and washing, PFA 2% was added to wells to fix cells for 10 min at 4° C. and analyzed on CytoFlex (Beckman) cytofluorometer to detect the inhibition of Fc-CLEC1-labelled.

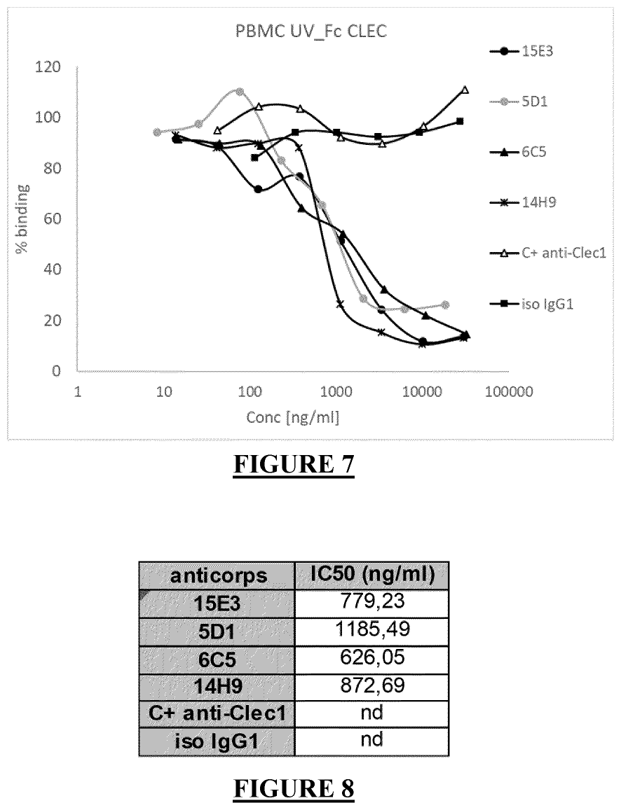

Results: FIGS. 4-8 illustrate the antagonistic activity of the murine and chimeric anti-hCLEC1 antibodies of the invention, compared to isotype control or in-house non-antagonist anti-CLEC1 control (control+ anti-Clecl). Fc-CLEC1 at 10 nM was able to bind specifically permeabilized Raji or apoptotic PBMC compared to Fc-isotype-A488 control (see FIGS. 4 and 5). Indeed, the 11 tested antibodies (encompassing murine and chimeric antibodies) were able to block interaction of Fc-CLEC to its ligands on permeabilized Raji or apoptotic PBMC in dose-dependent manner, compared to isotype control or non-antagonist antibody, which did not inhibit the binding of Fc-CLEC on cells. Among the 11 antibodies, IC50 were similar for all (see FIGS. 6 and 8), and inhibition profile curve were similar (see FIG. 7).

Accordingly, all the tested murine and chimeric antibodies of the invention are able to prevent the binding between CLEC-11 and cells usually binding to CLEC-1A, thereby illustrating that these antibodies are able to antagonize the binding between CLEC-1A and one of its ligand. Thus, this example illustrates that the antibodies of the invention are antagonist of human CLEC-1.

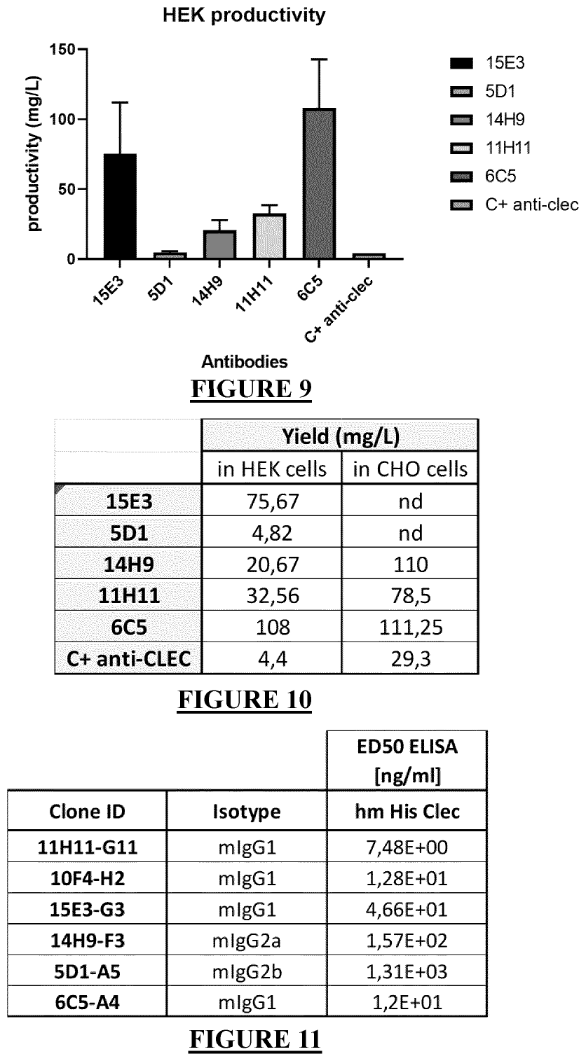

### Example 4: Production of Chimeric Anti-CLEC1 Antibodies—FIGS. 9 and 10

In mammalian HEK cells and in CHO cells, we have co-transfected, by lipofectamine method or by polyethylenimine (PEI), respectively, plasmids containing VH-hFcG4m with plasmid containing VL-CLkappa. After 5-6 days incubation, supernatant was recovered and quantified by sandwich ELISA assay. Supernatant could be purified by affinity on Protein A chromatography (HiTrap, GeHealthcare) with citric acid 0.1 M pH 3 elution buffer. Purified antibody was dialyzed in PBS and concentrated. They were quantified by UV (A280 nm) Antibodies of the invention were well expressed with different productivity as shown in FIGS. 9 and 10 (signal peptide used: IgKleader). As shown in FIGS. 9 and 10, 6C5 and 15E3 chimeric antibodies had high production yield in HEK cells, and 6C5 has a high production yield in CHO l cells. ND corresponds to absence of production assay for several antibodies. This example illustrates that the antibodies of the invention may be efficiently produced in recombinant production systems.

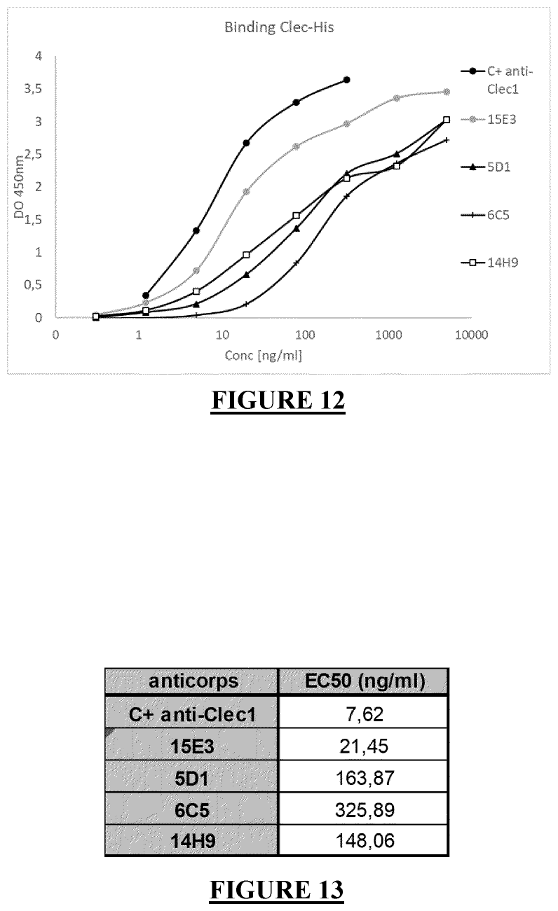

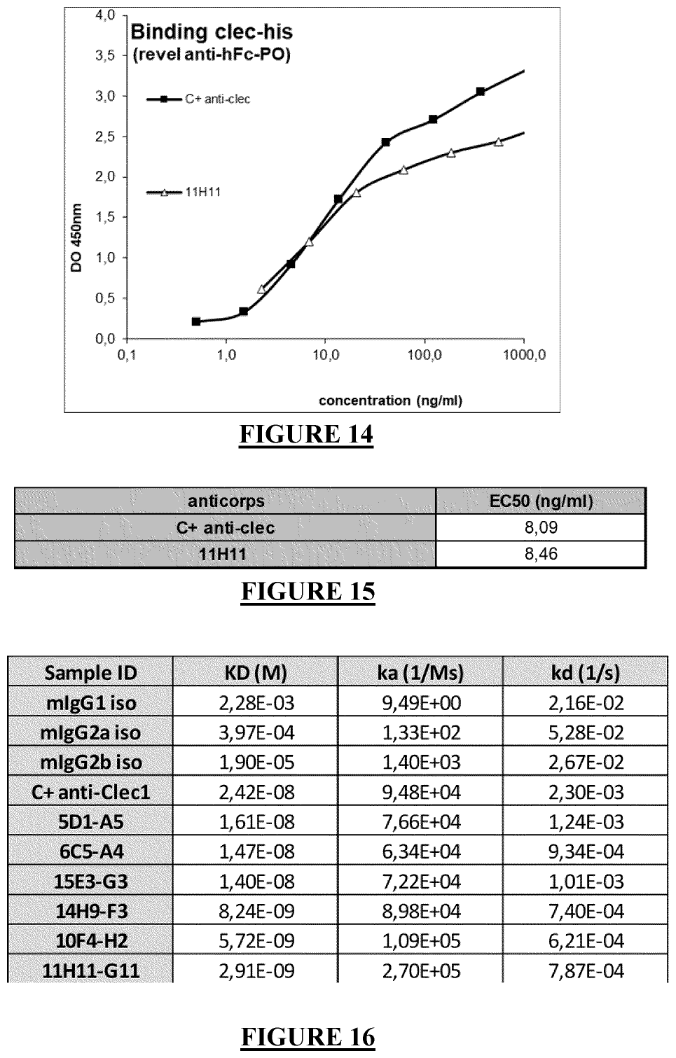

### Example 5. CLEC1 Bindings Assay of Monoclonal Anti-hCLEC1 Antibodies by ELISA—FIGS. 11-15

Method: The binding activity of the anti-hCLEC1 antibodies was assessed by ELISA (Enzyme-linked immunosorbent assay). For the ELISA assay, recombinant hCLEC1-His (R&D Systems reference 1704-CL) was immobilized on plastic at 2 μg/ml in carbonate buffer (pH9.2) and purified antibodies were added at different concentrations to measure binding. After incubation and washing, peroxidase-labelled donkey anti-mouse IgG chain (Jackson Immunoresearch; reference 715-036-151) was added and revealed by conventional methods. A second ELISA assays was performed, like above, with immobilization of CLEC1-Fc (OSE Immunotherapeutics) at 2 μg/ml. A third ELISA assays was performed to see the cross-reactivity with mouse CLEC1. Like above, ELISA was made with immobilization of mouse Fc-CLEC1 (OSE Immunotherapeutics) at 2 μg/ml in carbonate buffer instead of His-Clec. Purified antibodies were added at different concentrations to measure binding. After incubation and washing, goat anti mouse IgG kappa chain (Jackson Immunoresearch; reference 115-005-174) then peroxidase-labelled donkey anti-Goat IgG (Jackson Immunoresearch; reference 705-035-147) was added and revealed by conventional methods. Control antibody is a commercial anti-CLEC-1A antibody.

Results: As shown in FIGS. 11-15, the binding activity of different murine and chimeric anti-CLEC1 antibodies on CLEC1-His as measured by ELISA showed a binding activity for all antibodies with different ED50 or EC50. All murine and chimeric anti-CLEC-1A antibodies of the invention elicit a specific binding activity to CLEC-His. Binding activity of chimeric anti-CLEC1 antibodies on CLEC1-His as measured by ELISA showed a binding activity for all antibodies with different EC50. chimeric antibodies of the invention 15E3, 5D1, 14H9 and 6C5 had a good binding activity to CLEC-His (see FIGS. 13 and 15). This example demonstrates that the antibodies of the invention have a specific affinity for the human CLEC-1A.

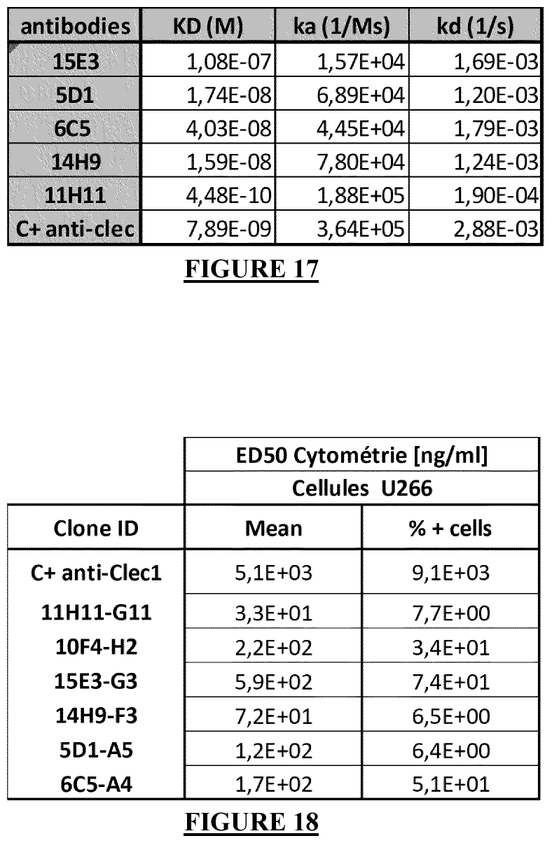

### Example 6 : Anti-hCLEC1 Antibodies Affinity Study—FIGS. 16 and 17

The affinity of the anti-hCLEC1 antibodies, was measured with Blitz system (Forté Bio, C22-2 No 61010-1). CLEC1-His recombinant protein (R&D Systems reference 1704-CL) was immobilized at 10 μg/ml by histidine tail into a Ni-NTA biosensor (Forté Bio, 18-0029) for 30 seconds.

Mouse anti-CLEC1 antibodies (FIG. 16) and chimeric anti-CLEC-1A antibodies (FIG. 17) were associated at 20 μg/mL (saturating concentration) for 120 seconds. The dissociation of antibodies was made in kinetics buffer for 120 seconds. Analysis was made with Blitz pro 1.2 software, which calculates association constant (ka) and dissociation constant (kd) and determined the affinity constant KD (ka/kd).

Results. Anti-CLEC1 antibodies (murine and chimeric) had a good affinity constant (range 2-10 nM) in Blitz, which was often 1-log inferior to biacore affinity analysis. Some antibodies like 21B1-E10 or 11H11-G11 had a good KD constant near 1 nM on HisCLEC protein, with a high association and low dissociation constant. The murine antibodies of the invention have all a KD value lower than the control antibody (see FIG. 16). Anti-CLEC1 chimeric antibodies had a affinity constant with range 10-100 nM) in Blitz, which was often 1-log inferior to biacore affinity analysis. Some antibodies like 5D1 or 14H9 conserved a KD affinity constant near 10 nM, like with murine clones, with a good association and low dissociation constant (see FIG. 17).

### Example 7: CLEC1 Binding Assay on Human U266 Cells Line to Compare Different Anti-hCLEC1 Antibodies, Including Murine Antibodies of the Invention, by Flow Cytometry—FIGS. 18

Method: To measure binding of anti-CLEC1 on human U266 cell lines (pre-blocked with Fcblock to blocking FcR), antibodies at different concentrations were incubated for 30 min at 4° C., and washed before stained 30 min at 4° C. with PE-labelled anti-mouseIgG (Jackson Immunoresearch; reference 715-116-151). Samples were analyzed on Cytoflex cytofluorometer (Beckman Coulter). The WI of anti-CLEC1 was compared between all anti-CLEC1 antibodies and percentage of stained cells was analysed in all conditions.

Results: The results indicate a strong binding (high MFI) of clones 14H9-F3 and 5D1-A5. Clones 11H11-G11, 10F4-H2, 6C5-A4 and 15E3-G3 bound good U266 CLEC1. As compared to the in-house control antibody, the ED50 is much lower for the antibodies of the invention as compared to this in-house control anti-CLEC-1A antibody.

The present murine antibodies of the invention are thereof much more affine for their target. This example illustrates that the murine antibodies of the invention bind specifically to human CLEC-1A expressed on the cell membrane of human cells, contrary to the in-house control anti-CLEC-1A antibody.

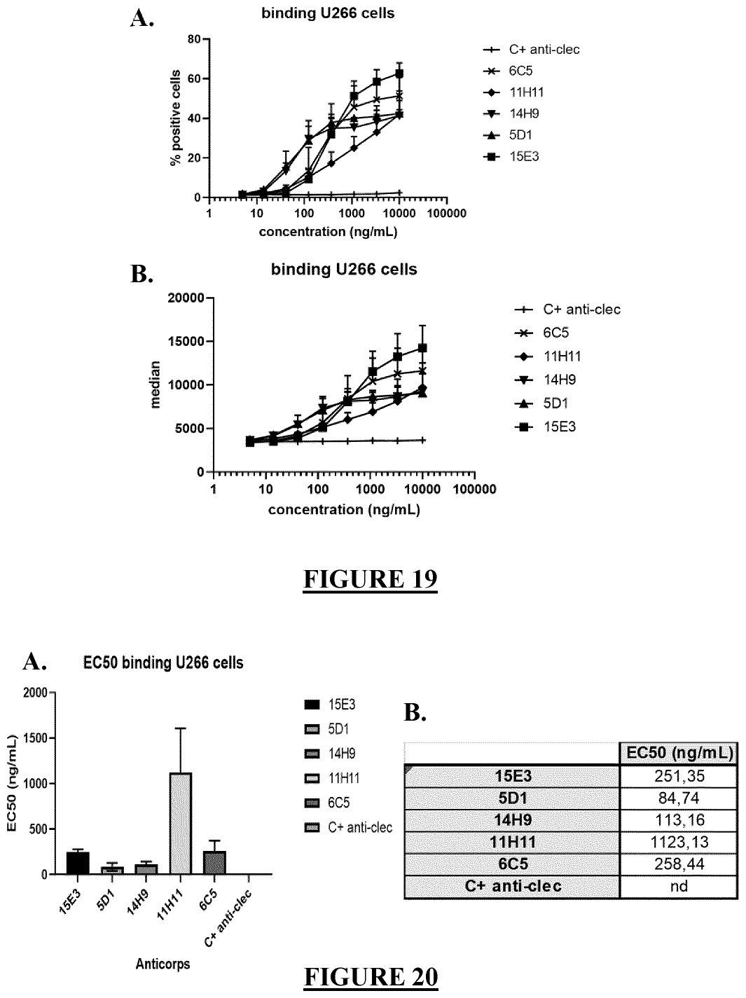

### Example 8: Clec1 Binding Assay on Human U266 Cells Line to Compare Chimeric Anti-hCLEC1 Antibodies by Flow Cytometry—FIGS. 19 and 20

Method: To measure binding of anti-CLEC1 on human U266 cell lines (pre-blocked with Fcblock to blocking FcR), antibodies at different concentrations were incubated for 30 min at 4° C., and washed before stained 30 min at 4° C. with PE-labelled anti-human IgG (Biolegend; reference 409304). Samples were analyzed on Cytoflex cytofluorometer (Beckman Coulter). The MFI of anti-CLEC1 was compared between all anti-CLEC1 antibodies and percentage of stained cells was analysed in all conditions.

Results: The results indicate a strong binding (high MFI and high percentage of positive cells) for all tested chimeric antibodies of the invention, similar to the result obtained with mouse anti-CLEC1 clones (14H9/5D1).

This example illustrates that the chimeric antibodies of the invention bind specifically to human CLEC-1A expressed on the cell membrane of human cells, contrary to the in-house control anti-CLEC-1A antibody.

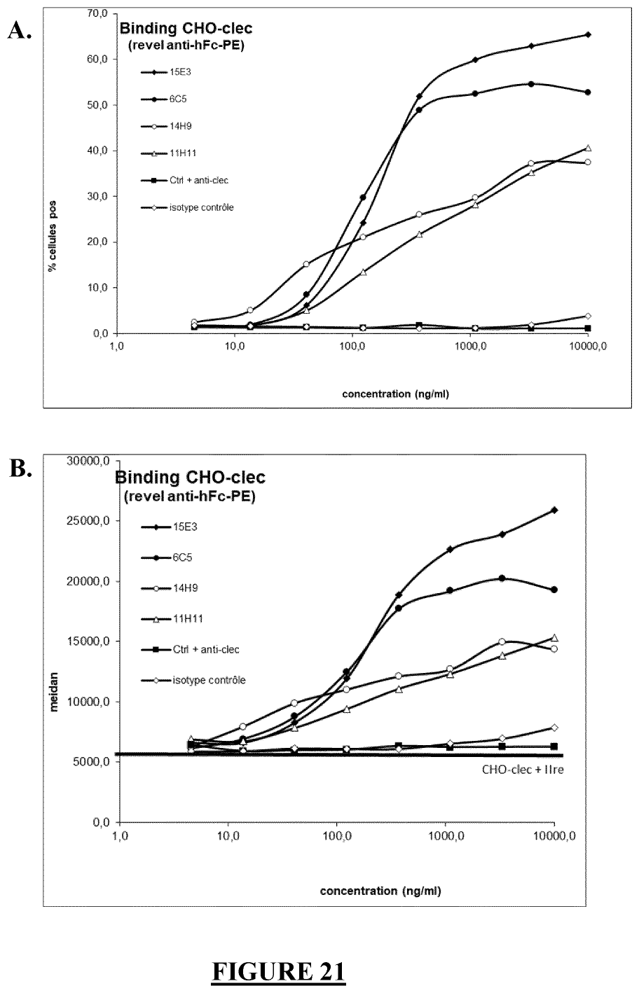

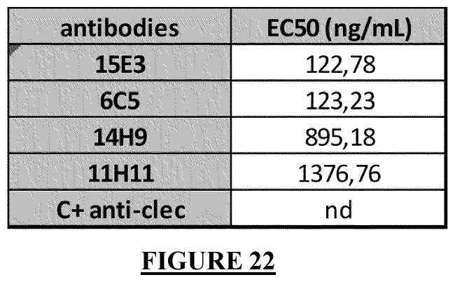

### Example 9. CLEC-1A Binding Assay on Human CHO Cells Line to Compare Chimeric Anti-hCLEC1 Antibodies by Flow Cytometry—FIGS. 21 and 22

Method: CLEC-1A binding assay on CHO-Clec-1 cells (cells transduced with a Clec-1 expressing lentivirus) by cytofluorometry

To measure binding of anti-clec on CHO-Clec-1 cells, human Fc Receptor Binding Inhibitor diluted at 1/200 (BD pharmingen; USA; reference 564220) was first added for 30 min at room-temperature to block human Fc receptors on CHO-Clec-1 cells to reduce background. Then, antibodies were incubated for 30 min at 4° C., and washed before stained 15 min at 4° C. with PE-labelled anti-human IgG Fc (Biolegend; USA; reference 409303). Samples were analyzed on citoflex (Beckman coulter)

Results. The results indicate a strong binding (high MFI and high percentage of positive cells) for all tested chimeric antibodies of the invention. This example illustrates that the chimeric antibodies of the invention bind specifically to human CLEC-1A expressed on the cell membrane of human cells, contrary to the in-house control anti-CLEC-1A antibody.

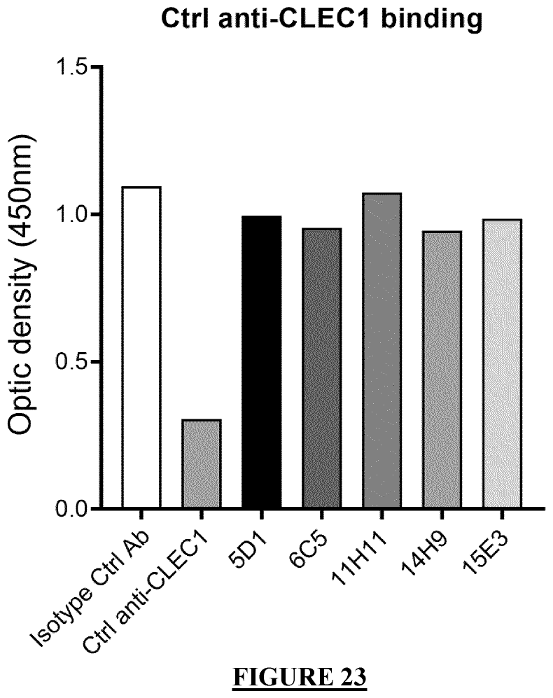

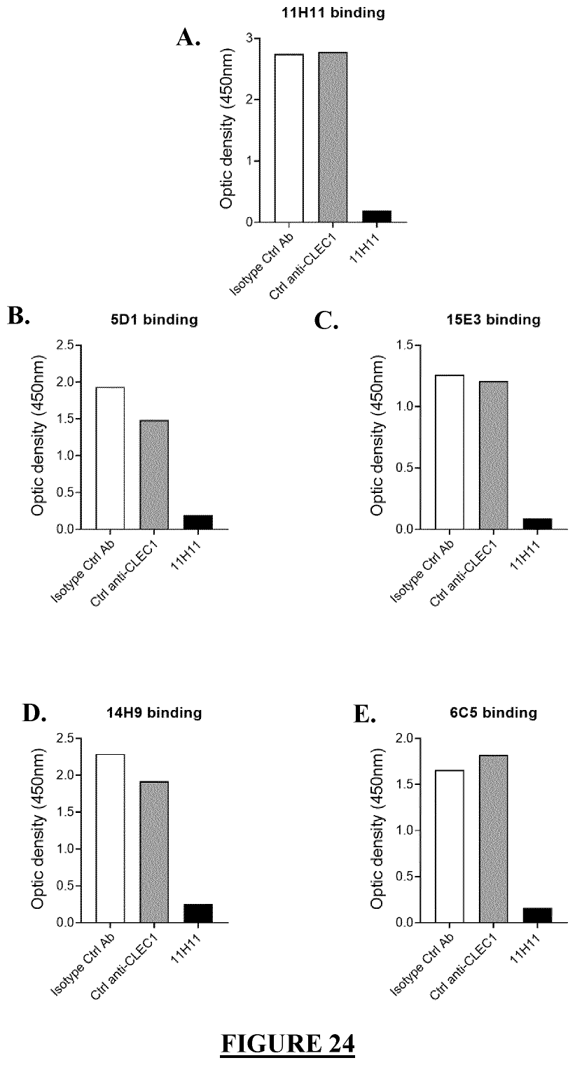

### Example 10. Cross-Competition for the Binding to His-CLEC-1A Between Different Anti-CLEC-1A Antibodies. FIGS. 23 and 24

Method. For competitive ELISA assay, recombinant His-CLEC1 (#1704-CL R&D Systems, Minneapolis, USA) was immobilized on plastic at 2 μg/ml in carbonate buffer (pH 9.2) and purified competitor antibodies or biotinylated challenged antibodies were added at 1 μg/mL or between 10 ng/mL to 2 μg/mL according the EC50 of biotinylated mAb respectively to measure binding to CLEC1. After incubation and washing, peroxidase-labelled streptavidin was added and revealed by conventional methods. Ctrl anti-CLEC1 antibody corresponds to the anti-CLEC-1A antibody disclosed in in WO2018073440A1 and Robles et al. (Blood advances, 2017).

Results. As illustrated on FIG. 23, the antibody of the prior art does not cross-compete for the binding to His-CLEC-1A with any antibody of the invention. Indeed, the binding of 5D1, 6C5, 11H11, 14H9 and 15E3 to His-CLEC-1A is not affected by the presence of the antibody of the prior art, in particular does not cross-compete with neither 5D1 nor 11H11. Accordingly, the antibodies of the invention does not bind to the same localization on CLEC-1A than the anti-CLEC-1A antibody of the prior art (disclosed in in WO2018073440A1 and Robles et al. (Blood advances, 2017)).

As illustrated on FIG. 24, wherein the cross-competition binding to His-CLEC-1A was performed between a selected antibody of the invention (11H11) and 5 antibodies of the invention (15E3, 5D1, 6C5, 14H9, 11H11). It is shown that the 5 tested antibodies cross-compete with 11H11 for the binding to His-CLEC-1A, illustrating that they all share a similar binding localization.

1. An antibody or antigen-binding fragment thereof that specifically binds to the extracellular domain of human C-type lectin-like receptor-1 member A receptor (CLEC-1A receptor) which comprises:
an antibody heavy chain variable domain comprising three VHCDRs wherein their amino acid sequences are respectively selected from:
VHCDR1 of SEQ ID No: 57; SEQ ID No: 65; SEQ ID No: 73; SEQ ID No: 81; SEQ ID No: 89 or SEQ ID No: 97; and
VHCDR2 of SEQ ID No: 59; SEQ ID No: 67; SEQ ID No: 75; SEQ ID No: 83; or SEQ ID No: 91; and
VHCDR3 of SEQ ID No: 61; SEQ ID No: 69; SEQ ID No: 77; SEQ ID No: 85 or SEQ ID No: 93; and
an antibody light chain variable domain comprising three VLCDRs wherein their amino acid sequence is selected from:
VLCDR1 of SEQ ID No: 4; SEQ ID No: 12; SEQ ID No: 20; SEQ ID No: 28 or SEQ ID No: 36; and
VLCDR2 of SEQ ID No: 6; SEQ ID No: 14; SEQ ID No: 22; SEQ ID No: 30 or SEQ ID No: 38; and
VLCDR3 of SEQ ID No: 8; SEQ ID No: 16; SEQ ID No: 24; SEQ ID No: 32 or SEQ ID No: 40;

2. The antibody or antigen-binding fragment thereof according to claim 1, which comprises at least one CDR domain selected from:
A VHCDR1 comprising the amino acid sequence set forth in SEQ ID No: 65, SEQ ID No: 81 or SEQ ID No: 97; and
A VHCDR2 comprising the amino acid sequence set forth in SEQ ID No. 67 or of SEQ ID No: 75 or of SEQ ID No: 83; and
A VHCDR3 comprising the amino acid sequence set forth in SEQ ID No. 69 or of SEQ ID No: 77 or of SEQ ID No: 85; and
A VLCDR1 comprising the amino acid sequence set forth in SEQ ID No. 12 or of SEQ ID No: 20 or of SEQ ID No: 28; and
A VLCDR2 comprising the amino acid sequence set forth in SEQ ID No. 14 or of SEQ ID No: 22 or of SEQ ID No: 30; and
A VLCDR3 comprising the amino acid sequence set forth in SEQ ID No. 16 or of SEQ ID No: 24 or of SEQ ID No: 32.

3. The antibody or antigen-binding fragment thereof according to claim 1, which antagonizes the binding of a fusion protein comprising the extracellular domain of human CLEC-1A receptor fused with a Fc fragment of a a human IgG, to secondary necrotic cells and/or tumor cells and/or to the intracellular content of secondary necrotic cells and/or tumor cells.

4. The antibody or antigen-binding fragment thereof according to claim 1, wherein the antibody heavy chain variable domain comprises the VHCDR1, VHCDR2 and VHCDR3 of sequence:
SEQ ID No: 57; SEQ ID No: 59 and SEQ ID No: 61 respectively; or
SEQ ID No: 65; SEQ ID No: 67 and SEQ ID No: 69 respectively; or
SEQ ID No: 73; SEQ ID No: 75 and SEQ ID No: 77 respectively; or
SEQ ID No: 81; SEQ ID No: 83 and SEQ ID No: 85 respectively; or
SEQ ID No: 89; SEQ ID No: 91 and SEQ ID No: 93 respectively; or
SEQ ID No: 97; SEQ ID No: 75 and SEQ ID No: 77 respectively.

5. The antibody or antigen-binding fragment thereof according to claim 1, wherein the antibody light chain variable domain comprises the VLCDR1, VLCDR2 and VLCDR3 of sequence:
SEQ ID No: 4; SEQ ID No: 6 and SEQ ID No: 8 respectively; or
SEQ ID No: 12; SEQ ID No: 14 and SEQ ID No: 16 respectively; or
SEQ ID No: 20; SEQ ID No: 22 and SEQ ID No: 24 respectively; or
SEQ ID No: 28; SEQ ID No: 30 and SEQ ID No: 32 respectively; or
SEQ ID No: 36; SEQ ID No: 38 and SEQ ID No: 40 respectively.

6. The antibody or antigen-binding fragment thereof according to claim 1, wherein
(a) the antibody heavy chain variable domain comprises the VHCDR1, VHCDR2 and VHCDR3 of sequence SEQ ID No: 57; SEQ ID No: 59 and SEQ ID No: 61 respectively, and wherein the antibody light chain variable domain comprises the VLCDR1, VLCDR2 and VLCDR3 of sequence SEQ ID No: 4; SEQ ID No: 6 and SEQ ID No: 8 respectively; or
(b) the antibody heavy chain variable domain comprises the VHCDR1, VHCDR2 and VHCDR3 of sequence SEQ ID No: 65; SEQ ID No: 67 and SEQ ID No: 69 respectively, and wherein the antibody light chain variable domain comprises the VLCDR1, VLCDR2 and VLCDR3 of sequence SEQ ID No: 12; SEQ ID No: 14 and SEQ ID No: 16 respectively; or
(c) the antibody heavy chain variable domain comprises the VHCDR1, VHCDR2 and VHCDR3 of sequence SEQ ID No: 73; SEQ ID No: 75 and SEQ ID No: 77 respectively, and wherein the antibody light chain variable domain comprises the VLCDR1, VLCDR2 and VLCDR3 of sequence SEQ ID No: 20; SEQ ID No:22 and SEQ ID No: 24 respectively; or
(d) the antibody heavy chain variable domain comprises the VHCDR1, VHCDR2 and VHCDR3 of sequence SEQ ID No: 81; SEQ ID No: 83 and SEQ ID No: 85 respectively, and wherein the antibody light chain variable domain comprises the VLCDR1, VLCDR2 and VLCDR3 of sequence SEQ ID No: 28; SEQ ID No 30 and SEQ ID No: 32 respectively; or
(e) the antibody heavy chain variable domain comprises the VHCDR1, VHCDR2 and VHCDR3 of sequence SEQ ID No: 89; SEQ ID No: 91 and SEQ ID No: 93 respectively, and wherein the antibody light chain variable domain comprises the VLCDR1, VLCDR2 and VLCDR3 of sequence SEQ ID No: 36; SEQ ID No: 38 and SEQ ID No: 40 respectively; or
(f) the antibody heavy chain variable domain comprises the VHCDR1, VHCDR2 and VHCDR3 of sequence SEQ ID No: 97; SEQ ID No: 75 and SEQ ID No: 77 respectively, and wherein the antibody light chain variable domain comprises the VLCDR1, VLCDR2 and VLCDR3 of sequence SEQ ID No: 20; SEQ ID No: 22 and SEQ ID No: 24 respectively.

7. The antibody or antigen-binding fragment thereof according to claim 1, wherein the antibody heavy chain variable domain comprises or consists of the amino acid sequence set forth in SEQ ID No: 55; SEQ ID No: 63; SEQ ID No: 71; SEQ ID No: 79; SEQ ID No: 87 or SEQ ID No: 95.

8. The antibody or antigen-binding fragment thereof according to claim 1, wherein the antibody light chain variable domain comprises the amino acid sequence set forth in SEQ ID No: 2; SEQ ID No: 10; SEQ ID No: 18; SEQ ID No: 26; SEQ ID No: 34 or SEQ ID No: 42.

9. The antibody or antigen-binding fragment thereof according to claim 1 comprising:
a heavy variable domain comprising the amino acid sequence set forth in SEQ ID No: 55 and a light variable domain comprising the amino acid sequence set forth in SEQ ID No: 2; or
a heavy variable domain comprising the amino acid sequence set forth in SEQ ID No: 63 and a light variable domain comprising the amino acid sequence set forth in SEQ ID No: 10; or
a heavy variable domain comprising the amino acid sequence set forth in SEQ ID No: 71 and a light variable domain comprising the amino acid sequence set forth in SEQ ID No: 18; or
a heavy variable domain comprising the amino acid sequence set forth in SEQ ID No: 79 and a light variable domain comprising the amino acid sequence set forth in SEQ ID No: 26; or
a heavy variable domain comprising the amino acid sequence set forth in SEQ ID No: 87 and a light variable domain comprising the amino acid sequence set forth in SEQ ID No: 34; or
a heavy variable domain comprising the amino acid sequence set forth in SEQ ID No: 95 and a light variable domain comprising the amino acid sequence set forth in SEQ ID No: 42.

10. The antibody or antigen-binding fragment thereof according to claim 1, wherein the antibody is a recombinant antibody, a chimeric antibody or a humanized antibody, an antibody that comprises a human IgG1, IgG2, IgG3 or IgG4 constant region.

11. The antibody or antigen-binding fragment thereof according to claim 1, which binds to human CLEC-1A with an affinity constant (KD) of at least 1E-07 M.

12. The antibody or antigen-binding fragment thereof according to claim 1, which, when used in vivo and/or in vitro, increases the phagocytosis of tumor cells by at least 10% as compared to a the negative control.

13. A nucleic acid molecule, or a combination of nucleic acid molecules, which encode(s) a polypeptide comprising or consisting of an antibody or antigen-binding fragment thereof according to claim 1, said nucleic acid molecule or combination of nucleic acid molecules comprising at least one nucleotide sequence selected from the group consisting of SEQ ID No: 1, SEQ ID No: 3, SEQ ID No: 5, SEQ ID No: 7, SEQ ID No: 9, SEQ ID No: 11, SEQ ID No: 13, SEQ ID No: 15, SEQ ID No: 17, SEQ ID No: 19, SEQ ID No: 21, SEQ ID No: 23, SEQ ID No: 25, SEQ ID No: 27, SEQ ID No: 29, SEQ ID No: 31, SEQ ID No: 33, SEQ ID No: 35, SEQ ID No: 37, SEQ ID No: 39, SEQ ID No: 41, SEQ ID No: 43, SEQ ID No: 44, SEQ ID No: 45, SEQ ID No: 56, SEQ ID No: 58, SEQ ID No: 60, SEQ ID No: 62, SEQ ID No: 64, SEQ ID No: 66, SEQ ID No: 68, SEQ ID No: 70, SEQ ID No: 72, SEQ ID No: 74, SEQ ID No: 76, SEQ ID No: 78, SEQ ID No: 80, SEQ ID No: 82, SEQ ID No: 84, SEQ ID No: 86, SEQ ID No: 88, SEQ ID No: 90, SEQ ID No: 92, SEQ ID No: 94, SEQ ID No: 96, SEQ ID No: 98 and/or SEQ ID No: 99, said nucleic acid molecule or combination or nucleic acid molecules encoding at least the 6 CDR domains of the antibody or antigen-binding fragment thereof.

14. A combination of compounds comprising a first therapeutic agent and at least one second therapeutic agent, wherein:
the first therapeutic agent is an antibody or antigen-binding fragment thereof according to claim 1
to the extracellular domain of human C-type lectin-like receptor-1 member A receptor (CLEC-1A receptor) and which competes with an antibody comprising or consisting of a heavy variable domain comprising or consisting of SEQ ID No. 71 and a light variable domain comprising or consisting of SEQ ID No. 18, in particular comprising or consisting of a heavy domain comprising or consisting of SEQ ID No. 121 and a light domain comprising or consisting of SEQ ID No. 128, for binding to a human CLEC-1A receptor, and which is an antagonist of human CLEC-1; or
an antibody or an antigen-binding fragment thereof, which specifically binds to the extracellular domain of human C-type lectin-like receptor-1 member A receptor (CLEC-1A receptor) and, which competes with an antibody comprising or consisting of a heavy variable domain comprising or consisting of SEQ ID No. 63 and a light variable domain comprising or consisting of SEQ ID No. 10, in particular comprising or consisting of a heavy domain comprising or consisting of SEQ ID No. 120 and a light domain comprising or consisting of SEQ ID No. 127, for binding to a human CLEC-1A receptor, and which is an antagonist of human CLEC-1; or an antibody or an antigen-binding fragment thereof, which specifically binds to the extracellular domain of human C-type lectin-like receptor-1 member A receptor (CLEC-1A receptor) and which, when used in vivo and/or in vitro, increases the phagocytosis of tumor cells by at least 10% as compared to the negative control; and
ii) the at least one second therapeutic agent is selected from the group consisting of a tumor-targeting antibody or antigen-binding fragment thereof, a tumor-targeting monoclonal antibody or antigen-binding fragment thereof, a tumor-targeting monoclonal antibody or antigen-binding fragment thereof which activates and/or enhances the phagocytosis capability of macrophages, a monoclonal antibody selected from the group consisting of alemtuzumab, atezolizumab, bevacizumab, cetuximab, herceptin, panitumumab, rituximab, trastuzumab, an anti-PDL-1 antibody and an anti-CD47 antibody, an antibody or monoclonal antibody selected from the group consisting of an anti-PD1 antibody and an anti-SIRPa antibody, a chemotherapeutic agent, a cytotoxic agent with anti-proliferative, pro-apoptotic, cell cycle arresting and/or differentiation inducing effect, a cytotoxic agent selected from the group consisting of a cytotoxic antibody, alkylating drugs, anthracyclines, antimetabolites, anti-microtubule agents, topoisomerase inhibitors, alkaloids, bleomycin, antineoplastic drugs, and cyclophosphamide.

15. A method for the prevention and/or the treatment of a human disease or a human disorder, comprising administering the antibody or antigen-binding fragment thereof according to claim 1 to a human to increase the phagocytosis capability by myeloid cells.

16. A method for the treatment of a disease or a condition comprising administering the antibody or antigen-binding fragment thereof according to claim 1 to a human wherein induction of phagocytosis in a patient improves or prevents the disease or condition.

17. A method for the treatment of a patient having a cancer, a liquid or a solid cancer, a lymphoma, a colorectal cancer, a mesothelioma or a hepatocarcinoma, an inflammatory disease, a chronic infection or sepsis comprising administering the antibody or antigen-binding fragment thereof according to claim 1 to the patient.

18. A method comprising a combination therapy, wherein a first medicament comprising a chemotherapeutic agent, a radiotherapy agent, an immunotherapeutic agent, a tumor-targeting monoclonal antibody, a cell therapy agents CAR-T cells, an immunosuppressive agent, a pro-apoptotic agent, an antibiotic, a targeted cancer therapy, and/or a probiotic for simultaneous, separated, or sequential administration, is administered to a patient in need thereof with a second medicament comprising the antibody or antigen-binding fragment thereof according to claim 1.

19. The method according to claim 18, wherein the immunotherapeutic agent is a tumor-targeting monoclonal antibody.

20. The method according to claim 18, wherein the cell therapy agent is a CAR-T cell.

21. The antibody or antigen-binding fragment thereof according to claim 1, which is an antagonist of human CLEC-1.


SEQUENCE LISTING (133 sequences)

SEQ ID NO: 1 - DNA, 324 bp, artificial sequence
  Synthetic sequence
gagatcgtgctgacccagtccccaaccacactggctgctagcccaggagagaagatcatcatcacatgctctgactccagctctatctccagcaactacctgcactggtatcagcagaagcccggcttctcccctaagctgctgatctacggcaccagcaatctggcctctggcgtgcctgctaggtttagcggctctggctccggcaccacatattccctgaccatcggcacaatggaggccgaggatgtggctacatactattgtcagcagggctcttccatcccacggaccttcggcggcggcacaaagctggagatcaag

SEQ ID NO: 2 - protein, 108 aa, artificial sequence
  Synthetic sequence
EIVLTQSPTTLAASPGEKIIITCSDSSSISSNYLHWYQQKPGFSPKLLIYGTSNLASGVPARFSGSGSGTTYSLTIGTMEAEDVATYYCQQGSSIPRTFGGGTKLEIK

SEQ ID NO: 3 - DNA, 36 bp, artificial sequence
  Synthetic sequence
tctgactccagctctatctccagcaactacctgcac

SEQ ID NO: 4 - protein, 12 aa, artificial sequence
  Synthetic sequence
SDSSSISSNYLH

SEQ ID NO: 5 - DNA, 21 bp, artificial sequence
  Synthetic sequence
ggcaccagcaatctggcctct

SEQ ID NO: 6 - protein, 7 aa, artificial sequence
  Synthetic sequence
GTSNLAS

SEQ ID NO: 7 - DNA, 27 bp, artificial sequence
  Synthetic sequence
cagcagggctcttccatcccacggacc

SEQ ID NO: 8 - protein, 9 aa, artificial sequence
  Synthetic sequence
QQGSSIPRT

SEQ ID NO: 9 - DNA, 321 bp, artificial sequence
  Synthetic sequence
gacatccagatgacccagtccccagcttccctgtccgcctctgtgggagagaccgtgacaatcacctgcagggccacagagaacatctactcctatctggcttggtaccagcagaagcagggcaagagcccccagttcctggtgtataatgccaagacactggctgagggcatgccttctcggttctccggaagcggatctggaacccagttttccctgaagatcaacatcctgcagccagaggattttggcacatactattgtcagcaccatttcggaacaccactgacctttggcgctggaaccaagctggagctgaag

SEQ ID NO: 10 - protein, 107 aa, artificial sequence
  Synthetic sequence
DIQMTQSPASLSASVGETVTITCRATENIYSYLAWYQQKQGKSPQFLVYNAKTLAEGMPSRFSGSGSGTQFSLKINILQPEDFGTYYCQHHFGTPLTFGAGTKLELK

SEQ ID NO: 11 - DNA, 33 bp, artificial sequence
  Synthetic sequence
agggccacagagaacatctactcctatctggct

SEQ ID NO: 12 - protein, 11 aa, artificial sequence
  Synthetic sequence
RATENIYSYLA

SEQ ID NO: 13 - DNA, 21 bp, artificial sequence
  Synthetic sequence
aatgccaagacactggctgag

SEQ ID NO: 14 - protein, 7 aa, artificial sequence
  Synthetic sequence
NAKTLAE

SEQ ID NO: 15 - DNA, 27 bp, artificial sequence
  Synthetic sequence
cagcaccatttcggaacaccactgacc

SEQ ID NO: 16 - protein, 9 aa, artificial sequence
  Synthetic sequence
QHHFGTPLT

SEQ ID NO: 17 - DNA, 318 bp, artificial sequence
  Synthetic sequence
gacatccagatgaaccagagcccatccagcctgagcgcctctctgggcgataccatcacaatcacctgccacgcttcccagaacatcaacgtgtggctgagctggtaccagcagaagcccggcaacatccctaagctgctgatctataaggcctctaatctgcatacaggagtgccatccaggttctccggaagcggatctggaaccggctttacactgaccatctcttccctgcagcccgaggacatcgctacatactattgtcagcagggccagtcttactggacattcggcggcggcaccaagctggagatcaag

SEQ ID NO: 18 - protein, 106 aa, artificial sequence
  Synthetic sequence
DIQMNQSPSSLSASLGDTITITCHASQNINVWLSWYQQKPGNIPKLLIYKASNLHTGVPSRFSGSGSGTGFTLTISSLQPEDIATYYCQQGQSYWTFGGGTKLEIK

SEQ ID NO: 19 - DNA, 33 bp, artificial sequence
  Synthetic sequence
cacgcttcccagaacatcaacgtgtggctgagc

SEQ ID NO: 20 - protein, 11 aa, artificial sequence
  Synthetic sequence
HASQNINVWLS

SEQ ID NO: 21 - DNA, 21 bp, artificial sequence
  Synthetic sequence
aaggcctctaatctgcataca

SEQ ID NO: 22 - protein, 7 aa, artificial sequence
  Synthetic sequence
KASNLHT

SEQ ID NO: 23 - DNA, 24 bp, artificial sequence
  Synthetic sequence
cagcagggccagtcttactggaca

SEQ ID NO: 24 - protein, 8 aa, artificial sequence
  Synthetic sequence
QQGQSYWT

SEQ ID NO: 25 - DNA, 333 bp, artificial sequence
  Synthetic sequence
gacatcgtgctgactcagagccctgcttcactggccgtctccctgggacagggtgctacaatttcttgccgcgcttcagaatccgtcgacaaccacggcttctcttttatgaattggttccagcagaagcccggccagccccctaagctgctgatctacgctgcttccaaccagggaagcggagtgccagctaggttctccggaagcggatctggaaccgatttttccctgaatatccatcccatggaggaggacgatacagctatgtatttttgtcagcagagtaaggaggtgccctggacttttggcggggggactaaactggaaatcaag

SEQ ID NO: 26 - protein, 111 aa, artificial sequence
  Synthetic sequence
DIVLTQSPASLAVSLGQGATISCRASESVDNHGFSFMNWFQQKPGQPPKLLIYAASNQGSGVPARFSGSGSGTDFSLNIHPMEEDDTAMYFCQQSKEVPWTFGGGTKLEIK

SEQ ID NO: 27 - DNA, 45 bp, artificial sequence
  Synthetic sequence
cgcgcttcagaatccgtcgacaaccacggcttctcttttatgaat

SEQ ID NO: 28 - protein, 15 aa, artificial sequence
  Synthetic sequence
RASESVDNHGFSFMN

SEQ ID NO: 29 - DNA, 21 bp, artificial sequence
  Synthetic sequence
gctgcttccaaccagggaagc

SEQ ID NO: 30 - protein, 7 aa, artificial sequence
  Synthetic sequence
AASNQGS

SEQ ID NO: 31 - DNA, 27 bp, artificial sequence
  Synthetic sequence
cagcagagtaaggaggtgccctggact

SEQ ID NO: 32 - protein, 9 aa, artificial sequence
  Synthetic sequence
QQSKEVPWT

SEQ ID NO: 33 - DNA, 336 bp, artificial sequence
  Synthetic sequence
gacgccgtgatgacccagacaccactgagcctgcccgtgtccctgggcgatcaggcttctatctcctgcaggtccagccagtccctggagaacagcaatggcaacacatacctgaattggtatctgcagaagccaggccagagcccccagctgctgatctacagggtgtctaaccggttctccggcgtgctggacaggttctccggctctggctccggcaccgatttcacactgaagatctctagggtggaggctgaggacctgggcgtgtacttctgtctgcaggtgacccacgtgccctatacatttggcggcggcaccaagctggagatcaag

SEQ ID NO: 34 - protein, 112 aa, artificial sequence
  Synthetic sequence
DAVMTQTPLSLPVSLGDQASISCRSSQSLENSNGNTYLNWYLQKPGQSPQLLIYRVSNRFSGVLDRFSGSGSGTDFTLKISRVEAEDLGVYFCLQVTHVPYTFGGGTKLEIK

SEQ ID NO: 35 - DNA, 48 bp, artificial sequence
  Synthetic sequence
aggtccagccagtccctggagaacagcaatggcaacacatacctgaat

SEQ ID NO: 36 - protein, 16 aa, artificial sequence
  Synthetic sequence
RSSQSLENSNGNTYLN

SEQ ID NO: 37 - DNA, 21 bp, artificial sequence
  Synthetic sequence
agggtgtctaaccggttctcc

SEQ ID NO: 38 - protein, 7 aa, artificial sequence
  Synthetic sequence
RVSNRFS

SEQ ID NO: 39 - DNA, 27 bp, artificial sequence
  Synthetic sequence
ctgcaggtgacccacgtgccctataca

SEQ ID NO: 40 - protein, 9 aa, artificial sequence
  Synthetic sequence
LQVTHVPYT

SEQ ID NO: 41 - DNA, 318 bp, artificial sequence
  Synthetic sequence
gacatccagatgaaccagagcccatccagcctgtctgcctccctgggcgataccatcacaatcacctgccacgctagccagaacatcaacgtgtggctgtcttggtaccagcagaagcccggcaacatccctaagctgctgatctataaggcctccaatctgcatacaggcgtgccaagcaggttcagcggatctggatccggaaccaccttcaccctgaccatcgactctctgcagcccgaggacatcgctacctactattgtcagcagggccagtcctactggacattcggcggcggcaccaagctggagatcaag

SEQ ID NO: 42 - protein, 106 aa, artificial sequence
  Synthetic sequence
DIQMNQSPSSLSASLGDTITITCHASQNINVWLSWYQQKPGNIPKLLIYKASNLHTGVPSRFSGSGSGTTFTLTIDSLQPEDIATYYCQQGQSYWTFGGGTKLEIK

SEQ ID NO: 43 - DNA, 33 bp, artificial sequence
  Synthetic sequence
cacgctagccagaacatcaacgtgtggctgtct

SEQ ID NO: 44 - DNA, 21 bp, artificial sequence
  Synthetic sequence
aaggcctccaatctgcataca

SEQ ID NO: 45 - DNA, 24 bp, artificial sequence
  Synthetic sequence
cagcagggccagtcctactggaca

SEQ ID NO: 46 - DNA, 339 bp, artificial sequence
  Synthetic sequence
gacatcgtgatgagccagtctccttccagcctggccgtgtccgtgggagagaaggtgaccctgacatgcaagagcacccagaacctgttctactctacaaaccagaagaattacctggcctggtatcagcagaagcccggccagagccctaagctgctgatctattgggcttctaccagggagtccggagtgccagaccggttcaccggatccggaagcggaacagacttcaccctgacaatctcttccgtgaaggccgaggatccagccgtgtactattgtcagcagtactatacctacccctggacatttggcggcggcacaaagctggagatcaag

SEQ ID NO: 47 - protein, 113 aa, artificial sequence
  Synthetic sequence
DIVMSQSPSSLAVSVGEKVTLTCKSTQNLFYSTNQKNYLAWYQQKPGQSPKLLIYWASTRESGVPDRFTGSGSGTDFTLTISSVKAEDPAVYYCQQYYTYPWTFGGGTKLEIK

SEQ ID NO: 48 - DNA, 51 bp, artificial sequence
  Synthetic sequence
aagagcacccagaacctgttctactctacaaaccagaagaattacctggcc

SEQ ID NO: 49 - protein, 17 aa, artificial sequence
  Synthetic sequence
KSTQNLFYSTNQKNYLA

SEQ ID NO: 50 - DNA, 21 bp, artificial sequence
  Synthetic sequence
tgggcttctaccagggagtcc

SEQ ID NO: 51 - protein, 7 aa, artificial sequence
  Synthetic sequence
WASTRES

SEQ ID NO: 52 - DNA, 27 bp, artificial sequence
  Synthetic sequence
cagcagtactatacctacccctggaca

SEQ ID NO: 53 - protein, 9 aa, artificial sequence
  Synthetic sequence
QQYYTYPWT

SEQ ID NO: 54 - DNA, 351 bp, artificial sequence
  Synthetic sequence
caggtgcagctgcagcagtccggaccagagctggtgaagcctggagcctccgtgaagatgagctgcaaggcttctggcttcacctttacagactacgtgatcgcttgggtgaaggtgaggaccggacagggactggagtggatcggagagatctatccaggctctggctccatctactataacgagaagttcaagggcaaggccaccctgacagctgataagtccagcaataccgcctacatgcagctgtcttccctgacaagcgaggactctgccgtgtacttctgtgctagctctaccgtggtggcttttgattattggggccagggcaccacactgacagtgtccagc

SEQ ID NO: 55 - protein, 117 aa, artificial sequence
  Synthetic sequence
QVQLQQSGPELVKPGASVKMSCKASGFTFTDYVIAWVKVRTGQGLEWIGEIYPGSGSIYYNEKFKGKATLTADKSSNTAYMQLSSLTSEDSAVYFCASSTVVAFDYWGQGTTLTVSS

SEQ ID NO: 56 - DNA, 15 bp, artificial sequence
  Synthetic sequence
gactacgtgatcgct

SEQ ID NO: 57 - protein, 5 aa, artificial sequence
  Synthetic sequence
DYVIA

SEQ ID NO: 58 - DNA, 51 bp, artificial sequence
  Synthetic sequence
gagatctatccaggctctggctccatctactataacgagaagttcaagggc

SEQ ID NO: 59 - protein, 17 aa, artificial sequence
  Synthetic sequence
EIYPGSGSIYYNEKFKG

SEQ ID NO: 60 - DNA, 24 bp, artificial sequence
  Synthetic sequence
tctaccgtggtggcttttgattat

SEQ ID NO: 61 - protein, 8 aa, artificial sequence
  Synthetic sequence
STVVAFDY

SEQ ID NO: 62 - DNA, 351 bp, artificial sequence
  Synthetic sequence
cagatccacctggtgcagtctggccccgagctgaagaagcctggcgagaccgtgaagatctcttgtaaggcctccggctacaccttcacaaactttggcatgaattgggtgaagcaggctccaggcaagggcctgaagtggatgggctggatcaacaccaatacaggcgagcccacatacgccgacgatttcaagggcaggttcgctttttccctggagacctccgccagcacagcttatctgcagatcaacaatctgaagaacgaggacaccgctacatacttctgcgctaggggagctccagcttggtttacctattggggccagggcaccctggtgacagtgagcgcc

SEQ ID NO: 63 - protein, 117 aa, artificial sequence
  Synthetic sequence
QIHLVQSGPELKKPGETVKISCKASGYTFTNFGMNWVKQAPGKGLKWMGWINTNTGEPTYADDFKGRFAFSLETSASTAYLQINNLKNEDTATYFCARGAPAWFTYWGQGTLVTVSA

SEQ ID NO: 64 - DNA, 15 bp, artificial sequence
  Synthetic sequence
aactttggcatgaat

SEQ ID NO: 65 - protein, 5 aa, artificial sequence
  Synthetic sequence
NFGMN

SEQ ID NO: 66 - DNA, 51 bp, artificial sequence
  Synthetic sequence
tggatcaacaccaatacaggcgagcccacatacgccgacgatttcaagggc

SEQ ID NO: 67 - protein, 17 aa, artificial sequence
  Synthetic sequence
WINTNTGEPTYADDFKG

SEQ ID NO: 68 - DNA, 24 bp, artificial sequence
  Synthetic sequence
ggagctccagcttggtttacctat

SEQ ID NO: 69 - protein, 8 aa, artificial sequence
  Synthetic sequence
GAPAWFTY

SEQ ID NO: 70 - DNA, 354 bp, artificial sequence
  Synthetic sequence
caggtgcagctgaaggagagcggaccaggactggtggctccatctcagtccctgagcatcacctgcacaatctccggcttcagcctgacctcttacggcgtgcactgggtgaggcagccacctggcaagggactggagtggctggtggtcatctggtccgacggcagcacaatctataactctgctctgaagtcccggctgtctatctccaaggataacagcaagtctcaggtgttcctgaagatgaattctctgcagaccgacgatacagccatgtactattgtgctaggcatggcggctactataattactttgactattggggccagggcaccacactgaccgtgtccagc

SEQ ID NO: 71 - protein, 118 aa, artificial sequence
  Synthetic sequence
QVQLKESGPGLVAPSQSLSITCTISGFSLTSYGVHWVRQPPGKGLEWLVVIWSDGSTIYNSALKSRLSISKDNSKSQVFLKMNSLQTDDTAMYYCARHGGYYNYFDYWGQGTTLTVSS

SEQ ID NO: 72 - DNA, 15 bp, artificial sequence
  Synthetic sequence
tcttacggcgtgcac

SEQ ID NO: 73 - protein, 5 aa, artificial sequence
  Synthetic sequence
SYGVH

SEQ ID NO: 74 - DNA, 48 bp, artificial sequence
  Synthetic sequence
gtcatctggtccgacggcagcacaatctataactctgctctgaagtcc

SEQ ID NO: 75 - protein, 16 aa, artificial sequence
  Synthetic sequence
VIWSDGSTIYNSALKS

SEQ ID NO: 76 - DNA, 30 bp, artificial sequence
  Synthetic sequence
catggcggctactataattactttgactat

SEQ ID NO: 77 - protein, 10 aa, artificial sequence
  Synthetic sequence
HGGYYNYFDY

SEQ ID NO: 78 - DNA, 351 bp, artificial sequence
  Synthetic sequence
caggtgcagctgcagcagtccggaccagagctggtgaagcctggagcctctgtgaagatgtcctgcaaggctagcggctacaccttcacagactatgtgatctcttgggtgaagcagaagaccggacagggactggagtggatcggagagatctacccaggctccggcaacacatactataatgagaagtttaagggcaaggccaccctgacagctgataagtccagctctaccgcctatatccacctgtccagcctgacatccgaggacagcgccgtgtacttctgtgctggcggcggctcttcccattttgattattggggccagggcaccacactgaccgtgagctct

SEQ ID NO: 79 - protein, 117 aa, artificial sequence
  Synthetic sequence
QVQLQQSGPELVKPGASVKMSCKASGYTFTDYVISWVKQKTGQGLEWIGEIYPGSGNTYYNEKFKGKATLTADKSSSTAYIHLSSLTSEDSAVYFCAGGGSSHFDYWGQGTTLTVSS

SEQ ID NO: 80 - DNA, 15 bp, artificial sequence
  Synthetic sequence
gactatgtgatctct

SEQ ID NO: 81 - protein, 5 aa, artificial sequence
  Synthetic sequence
DYVIS

SEQ ID NO: 82 - DNA, 51 bp, artificial sequence
  Synthetic sequence
gagatctacccaggctccggcaacacatactataatgagaagtttaagggc

SEQ ID NO: 83 - protein, 17 aa, artificial sequence
  Synthetic sequence
EIYPGSGNTYYNEKFKG

SEQ ID NO: 84 - DNA, 24 bp, artificial sequence
  Synthetic sequence
ggcggctcttcccattttgattat

SEQ ID NO: 85 - protein, 8 aa, artificial sequence
  Synthetic sequence
GGSSHFDY

SEQ ID NO: 86 - DNA, 354 bp, artificial sequence
  Synthetic sequence
caggtgcagctgcagcagtctggaaccgagctggctagaccaggagcttctgtgaagatgtcctgcaaggccagcggctacatcttcaccgactatacaatccactgggtgaagcagaggccaggacagggactggagtgggtgggctacatcaacccttccagcggctacacaaactataatcagaagtttaaggccaaggctaccctgacagccgataagtcttccagcaccgcttatatgcagctgtcttccctgacatccgaggactccgccgtgtactattgtacccccatgttcaggcggtcctactttgattattggggccagggcaccacactgacagtgagctct

SEQ ID NO: 87 - protein, 118 aa, artificial sequence
  Synthetic sequence
QVQLQQSGTELARPGASVKMSCKASGYIFTDYTIHWVKQRPGQGLEWVGYINPSSGYTNYNQKFKAKATLTADKSSSTAYMQLSSLTSEDSAVYYCTPMFRRSYFDYWGQGTTLTVSS

SEQ ID NO: 88 - DNA, 15 bp, artificial sequence
  Synthetic sequence
gactatacaatccac

SEQ ID NO: 89 - protein, 5 aa, artificial sequence
  Synthetic sequence
DYTIH

SEQ ID NO: 90 - DNA, 51 bp, artificial sequence
  Synthetic sequence
tacatcaacccttccagcggctacacaaactataatcagaagtttaaggcc

SEQ ID NO: 91 - protein, 17 aa, artificial sequence
  Synthetic sequence
YINPSSGYTNYNQKFKA

SEQ ID NO: 92 - DNA, 27 bp, artificial sequence
  Synthetic sequence
atgttcaggcggtcctactttgattat

SEQ ID NO: 93 - protein, 9 aa, artificial sequence
  Synthetic sequence
MFRRSYFDY

SEQ ID NO: 94 - DNA, 354 bp, artificial sequence
  Synthetic sequence
caggtgcagctgaaggagtccggaccaggactggtggctccatctcagtccctgagcatcacctgcacaatctccggcttcagcctgaccacatacggcatccactgggtgaggcagccacctggcaagggactggagtggctggtggtcatctggtctgacggctccaccatctataactccgccctgaagtctcggctgtctatctccaaggataacagcaagtctcaggtgttcctgaagatgaattccctgcagaccgacgatacagccatctactattgtgctaggcatggcggctactataattactttgactattggggccagggcaccacactgacagtgtccagc

SEQ ID NO: 95 - protein, 118 aa, artificial sequence
  Synthetic sequence
QVQLKESGPGLVAPSQSLSITCTISGFSLTTYGIHWVRQPPGKGLEWLVVIWSDGSTIYNSALKSRLSISKDNSKSQVFLKMNSLQTDDTAIYYCARHGGYYNYFDYWGQGTTLTVSS

SEQ ID NO: 96 - DNA, 15 bp, artificial sequence
  Synthetic sequence
acatacggcatccac

SEQ ID NO: 97 - protein, 5 aa, artificial sequence
  Synthetic sequence
TYGIH

SEQ ID NO: 98 - DNA, 48 bp, artificial sequence
  Synthetic sequence
gtcatctggtctgacggctccaccatctataactccgccctgaagtct

SEQ ID NO: 99 - DNA, 30 bp, artificial sequence; identical to SEQ ID NO: 76
  Synthetic sequence

SEQ ID NO: 100 - DNA, 354 bp, artificial sequence; identical to SEQ ID NO: 94
  Synthetic sequence

SEQ ID NO: 101 - protein, 118 aa, artificial sequence; identical to SEQ ID NO: 95
  Synthetic sequence

SEQ ID NO: 102 - DNA, 15 bp, artificial sequence
  Synthetic sequence
acatacggcatccac

SEQ ID NO: 103 - protein, 5 aa, artificial sequence
  Synthetic sequence
TYGIH

SEQ ID NO: 104 - DNA, 48 bp, artificial sequence; identical to SEQ ID NO: 98
  Synthetic sequence

SEQ ID NO: 105 - protein, 16 aa, artificial sequence
  Synthetic sequence
VIWSDGSTIYNSALKS

SEQ ID NO: 106 - DNA, 30 bp, artificial sequence; identical to SEQ ID NO: 76
  Synthetic sequence

SEQ ID NO: 107 - protein, 10 aa, artificial sequence
  Synthetic sequence
HGGYYNYFDY

SEQ ID NO: 108 - protein, 206 aa, artificial sequence
  Synthetic sequence
YYQLSNTGQDTISQMEERLGNTSQELQSLQVQNIKLAGSLQHVAEKLCRELYNKAGAHRCSPCTEQWKWHGDNCYQFYKDSKSWEDCKYFCLSENSTMLKINKQEDLEFAASQSYSEFFYSYWTGLLRPDSGKAWLWMDGTPFTSELFHIIIDVTSPRSRDCVAILNGMIFSKDCKELKRCVCERRAGMVKPESLHVPPETLGEGD

SEQ ID NO: 109 - protein, 280 aa, artificial sequence
  Synthetic sequence
MQAKYSSTRDMLDDDGDTTMSLHSQGSATTRHPEPRRTEHRAPSSTWRPVALTLLTLCLVLLIGLAALGLLFFQYYQLSNTGQDTISQMEERLGNTSQELQSLQVQNIKLAGSLQHVAEKLCRELYNKAGAHRCSPCTEQWKWHGDNCYQFYKDSKSWEDCKYFCLSENSTMLKINKQEDLEFAASQSYSEFFYSYWTGLLRPDSGKAWLWMDGTPFTSELFHIIIDVTSPRSRDCVAILNGMIFSKDCKELKRCVCERRAGMVKPESLHVPPETLGEGD

SEQ ID NO: 110 - protein, 451 aa, artificial sequence
  Synthetic sequence
METDTLLLWVLLLWVPGSTGDVECPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKQYYQLSNTGQDTISQMEERLGNTSQELQSLQVQNIKLAGSLQHVAEKLCRELYNKAGAHRCSPCTEQWKWHGDNCYQFYKDSKSWEDCKYFCLSENSTMLKINKQEDLEFAASQSYSEFFYSYWTGLLRPDSGKAWLWMDGTPFTSELFHIIIDVTSPRSRDCVAILNGMIFSKDCKELKRCVCERRAGMVKPESLHVPPETLGEGD

SEQ ID NO: 111 - protein, 107 aa, artificial sequence [sequence omitted]

SEQ ID NO: 112 - DNA, 324 bp, artificial sequence
  Synthetic sequence
cgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 113 - protein, 330 aa, artificial sequence [sequence omitted]

SEQ ID NO: 114 - protein, 326 aa, artificial sequence
  Synthetic sequence
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 115 - protein, 327 aa, artificial sequence
  Synthetic sequence
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 116 - protein, 330 aa, artificial sequence
  Synthetic sequence
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIAKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 117 - protein, 327 aa, artificial sequence
  Synthetic sequence
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 118 - DNA, 984 bp, artificial sequence
  Synthetic sequence
gctagcaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctccgggtaaatga

SEQ ID NO: 119 - protein, 444 aa, artificial sequence
  Synthetic sequence
QVQLQQSGPELVKPGASVKMSCKASGFTFTDYVIAWVKVRTGQGLEWIGEIYPGSGSIYYNEKFKGKATLTADKSSNTAYMQLSSLTSEDSAVYFCASSTVVAFDYWGQGTTLTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 120 - protein, 444 aa, artificial sequence
  Synthetic sequence
QIHLVQSGPELKKPGETVKISCKASGYTFTNFGMNWVKQAPGKGLKWMGWINTNTGEPTYADDFKGRFAFSLETSASTAYLQINNLKNEDTATYFCARGAPAWFTYWGQGTLVTVSAASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 121 - protein, 445 aa, artificial sequence
  Synthetic sequence
QVQLKESGPGLVAPSQSLSITCTISGFSLTSYGVHWVRQPPGKGLEWLVVIWSDGSTIYNSALKSRLSISKDNSKSQVFLKMNSLQTDDTAMYYCARHGGYYNYFDYWGQGTTLTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 122 - protein, 444 aa, artificial sequence
  Synthetic sequence
QVQLQQSGPELVKPGASVKMSCKASGYTFTDYVISWVKQKTGQGLEWIGEIYPGSGNTYYNEKFKGKATLTADKSSSTAYIHLSSLTSEDSAVYFCAGGGSSHFDYWGQGTTLTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 123 - protein, 445 aa, artificial sequence
  Synthetic sequence
QVQLQQSGTELARPGASVKMSCKASGYIFTDYTIHWVKQRPGQGLEWVGYINPSSGYTNYNQKFKAKATLTADKSSSTAYMQLSSLTSEDSAVYYCTPMFRRSYFDYWGQGTTLTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 124 - protein, 445 aa, artificial sequence
  Synthetic sequence
QVQLKESGPGLVAPSQSLSITCTISGFSLTTYGIHWVRQPPGKGLEWLVVIWSDGSTIYNSALKSRLSISKDNSKSQVFLKMNSLQTDDTAIYYCARHGGYYNYFDYWGQGTTLTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 125 - protein, 445 aa, artificial sequence; identical to SEQ ID NO: 124
  Synthetic sequence

SEQ ID NO: 126 - protein, 215 aa, artificial sequence
  Synthetic sequence
EIVLTQSPTTLAASPGEKIIITCSDSSSISSNYLHWYQQKPGFSPKLLIYGTSNLASGVPARFSGSGSGTTYSLTIGTMEAEDVATYYCQQGSSIPRTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 127 - protein, 214 aa, artificial sequence
  Synthetic sequence
DIQMTQSPASLSASVGETVTITCRATENIYSYLAWYQQKQGKSPQFLVYNAKTLAEGMPSRFSGSGSGTQFSLKINILQPEDFGTYYCQHHFGTPLTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 128 - protein, 213 aa, artificial sequence
  Synthetic sequence
DIQMNQSPSSLSASLGDTITITCHASQNINVWLSWYQQKPGNIPKLLIYKASNLHTGVPSRFSGSGSGTGFTLTISSLQPEDIATYYCQQGQSYWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 129 - protein, 218 aa, artificial sequence
  Synthetic sequence
DIVLTQSPASLAVSLGQGATISCRASESVDNHGFSFMNWFQQKPGQPPKLLIYAASNQGSGVPARFSGSGSGTDFSLNIHPMEEDDTAMYFCQQSKEVPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 130 - protein, 219 aa, artificial sequence
  Synthetic sequence
DAVMTQTPLSLPVSLGDQASISCRSSQSLENSNGNTYLNWYLQKPGQSPQLLIYRVSNRFSGVLDRFSGSGSGTDFTLKISRVEAEDLGVYFCLQVTHVPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 131 - protein, 213 aa, artificial sequence
  Synthetic sequence
DIQMNQSPSSLSASLGDTITITCHASQNINVWLSWYQQKPGNIPKLLIYKASNLHTGVPSRFSGSGSGTTFTLTIDSLQPEDIATYYCQQGQSYWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 132 - protein, 220 aa, artificial sequence
  Synthetic sequence
DIVMSQSPSSLAVSVGEKVTLTCKSTQNLFYSTNQKNYLAWYQQKPGQSPKLLIYWASTRESGVPDRFTGSGSGTDFTLTISSVKAEDPAVYYCQQYYTYPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 133 - DNA, 1374 bp, artificial sequence
  Synthetic sequence
ctcgaggccaccatggaaaccgatacactgctgctgtgggtcctgctgctgtgggtgcctgggtcaactggagacgtggaatgtcccccttgtcccgcaccacctgtggctggaccttccgtgttcctgtttccacccaagccaaaggacaccctgatgatctctaggacccctgaggtgacatgcgtggtggtggacgtgtcccacgaggatccagaggtgaagttcaactggtacgtggatggcgtggaggtgcataatgctaagacaaagcccagggaggagcagtacaactctacctatcgggtggtgtccgtgctgacagtgctgcaccaggactggctgaacggcaaggagtataagtgcaaggtgagcaataagggcctgccttccagcatcgagaagaccatctctaaggctaagggacagccaagggagccacaggtgtacacactgcctccaagccgggaggagatgaccaagaaccaggtgtctctgacatgtctggtgaagggcttctatccctccgacatcgctgtggagtgggagagcaatggccagcctgagaacaattacaagaccacaccccctgtgctggactccgatggcagcttctttctgtatagcaagctgaccgtggataagtccaggtggcagcagggcaacgtgttttcttgttccgtgatgcacgaggccctgcacaatcattacacacagaagagcctgtctctgtccccaggcaagcagtactatcagctgtctaataccggccaggatacaatctcccagatggaggagcgcctgggcaacacctcccaggagctgcagagcctgcaggtgcagaatatcaagctggccggctccctgcagcatgtggctgagaagctgtgcagagagctgtacaacaaggctggagctcacaggtgcagcccatgtacagagcagtggaagtggcatggcgacaattgttaccagttctataaggacagcaagtcttgggaggattgcaagtacttttgtctgtctgagaactccaccatgctgaagatcaataagcaggaggatctggagttcgccgcttcccagagctattctgagttcttttactcctattggacaggcctgctgaggccagacagcggcaaggcttggctgtggatggatggcacccccttcacatctgagctgtttcatatcatcatcgacgtgaccagccccagatctcgcgattgcgtggccatcctgaacggcatgatcttttccaaggactgtaaggagctgaagaggtgcgtgtgcgagaggcgggctggtatggtgaagcccgagagcctgcatgtgcctcctgaaacactgggggaaggggactagtctaga